(19) United States
(12) Patent Application Publication
(10) Pub. No.: US 2020/0155490 A1
(43) Pub. Date: May 21, 2020

(54) IMMUNOMOUDULATORY AGENTS AND METHODS OF USE THEREOF

(71) Applicants: Maxim Artyomov, Kirkwood, MO (US); Vicky Lampropoulou, St. Louis, MO (US)

(72) Inventors: Maxim Artyomov, Kirkwood, MO (US); Vicky Lampropoulou, St. Louis, MO (US)

(21) Appl. No.: 15/998,551

(22) PCT Filed: Feb. 14, 2017
(86) PCT No.: PCT/US2017/017766

§ 371 Date: Aug. 15, 2018

Related U.S. Application Data
(60) Provisional application No. 62/295,348, filed on Feb. 15, 2016.

(51) Int. Cl. A61K 31/194; A61P 29/00

(52) U.S. Cl. CPC A61K 31/194; A61P 29/00

(57) ABSTRACT

The disclosure provides a method of suppressing an immune response comprising administration of an immunomodulatory agent. The disclosure further provides for an immunomodulatory agent comprising itaconate, malonate, or a derivative thereof. Further, the disclosure provides for a method of reducing the extent of tissue injury in ischemia reperfusion, including cardiovascular infarction comprising administration of an immunomodulatory agent and treating psoriasis.

### CROSS-REFERENCE TO RELATED APPLICATIONS

This application claims priority from U.S. Provisional Application Ser. No. 62/295,348 filed on 15 Feb. 2016, which is incorporated herein by reference in its entirety.

### STATEMENT REGARDING FEDERALLY SPONSORED RESEARCH OR DEVELOPMENT

Not Applicable.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure generally relates to a method of suppressing immune response. For example, the provided compositions and methods can comprise exogenously adding an immunomodulatory agent (e.g., itaconate derivative) to immune cells. Further, the disclosure provides administration of dimethyl itaconate to reduce the extent of tissue injury in cardiovascular infarction and psoriasis.

### BACKGROUND OF THE INVENTION

Remodeling of the tricarboxylic acid (TCA) cycle is a metabolic adaptation mechanism accompanying inflammatory macrophage activation. During this process, endogenous metabolites can adopt regulatory roles that govern specific aspects of inflammatory response. One of the most significant metabolic signals comes from succinate, which regulates the downstream pro-inflammatory IL-1β-HIF-1a axis. At present, the regulatory mechanisms modulating succinate levels remain unknown.

### SUMMARY OF THE INVENTION

Among the various aspects of the present disclosure is the provision of an immunomodulatory agent and uses thereof. In some embodiments, the method includes the provision of a method of treatment of a disease, disorder, or condition associated with an inflammatory response or an immune response.

Another aspect of the invention provides for a method to suppress an LPS-mediated immune response, the method comprising administering an immunomodulatory agent comprising itaconate, malonate, or a derivative thereof

Another aspect of the invention provides for a method to reduce tissue injury during cardiovascular infarction, the method comprising administering an immunomodulatory agent comprising itaconate, malonate, or a derivative thereof

In some embodiments, the itaconate, malonate, or a derivative thereof comprises a compound of formula I:

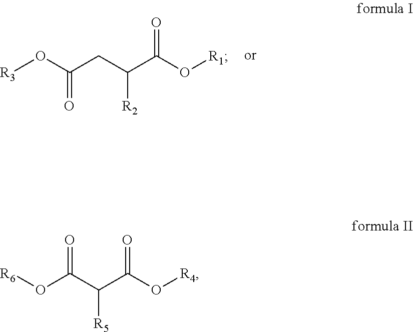

or a pharmaceutically acceptable salt thereof, including all tautomers and stereoisomers thereof wherein,

R₁ is hydrogen, unsubstituted or substituted alkyl; unsubstituted or substituted alkenes; or unsubstituted or substituted alkynes;

R₂ is hydrogen, unsubstituted or substituted alkyl; unsubstituted or substituted alkenes; or unsubstituted or substituted alkynes;

R₃ is hydrogen, unsubstituted or substituted alkyl; unsubstituted or substituted alkenes; or unsubstituted or substituted alkynes;

R₄ is hydrogen, unsubstituted or substituted alkyl; unsubstituted or substituted alkenes; or unsubstituted or substituted alkynes;

R₅ is hydrogen, unsubstituted or substituted alkyl; unsubstituted or substituted alkenes; or unsubstituted or substituted alkynes; and

R₆ is hydrogen, unsubstituted or substituted alkyl; unsubstituted or substituted alkenes; or unsubstituted or substituted alkynes;

wherein,
R₁, R₂, R₃, R₄, and R₅ is optionally substituted with one or more groups independently selected from the group consisting of hydroxyl; hydroxyl; amine; C₁₋₁₀carboxylic acid; C₁₋₁₀carboxyl, straight chain or branched C₁₋₁₀alkyl, optionally containing unsaturation; a C₂₋₆ cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl;
the unsubstituted phenyl ring or substituted phenyl ring is optionally substituted with one or more groups independently selected from the group consisting of hydroxyl; hydroxyl; amine; C₁₋₁₀carboxylic acid; C₁₋₁₀carboxyl, straight chain or branched C₁₋₁₀alkyl, optionally containing unsaturation; straight chain or branched C₁₋₁₀alkyl amine, optionally containing unsaturation; a C₂₋₆ cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and

the unsubstituted heterocyclyl or substituted heterocyclyl is optionally substituted with one or more groups independently selected from the group consisting of hydroxyl; hydroxyl; amine; C₁₋₁₀carboxylic acid; C₁₋₁₀carboxyl, straight chain or branched C₁₋₁₀alkyl, optionally containing unsaturation; straight chain or branched C₁₋₁₀alkyl amine, optionally containing unsaturation; a C₂₋₆ cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms. In some embodiments, R₁ is H or CH₃, R₂ is CH₂, CH₃, or H; R₃ is H or CH₃, R₄ is H or CH₃, R₅ is H or CH₃, and R₆ is H or CH₃.

In some embodiments, the itaconate or malonate, or a derivative thereof is selected from the group consisting of:

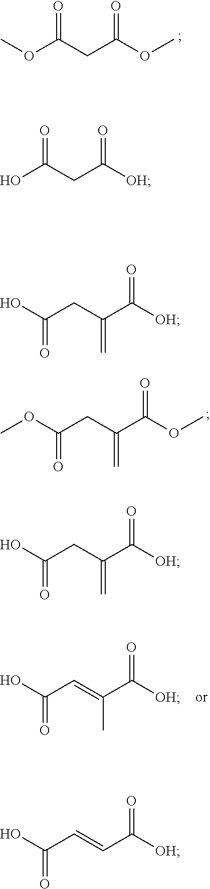

or a pharmaceutically acceptable salt thereof, including all tautomers and stereoisomers.

In some embodiments, the immunomodulatory agent downregulates proinflammatory pathways or upregulate Phase II conjugation, glutathione conjugation, or biological oxidations.

In some embodiments, the disease, disorder, or condition is selected from the group consisting of ischemia-reperfusion injury or an immune response.

In some embodiments, the immune response is an autoimmune response or a lipopolysaccharide (LPS)-mediated immune response; or the immunomodulatory agent interferes with (i) activation of pro-inflammatory macrophages, (ii) ROS-related oxidative stress, (iii) inflammatory T cell response, (iv) pathogenic adaptive immune response; (v) IL-17, or (vi) GM-CSF-production; and TNF-α production is substantially unaffected.

In some embodiments, the disease, disorder, or condition is associated with increased expression or increased secretion of Casp1, HIF-1α, pro-IL-1β, ASC, NLRP3, NOS2, iNOS, NO, 1L6, IFNβ1, IL-12p70, IL-6, IL-1β, IL-12β, GM-CSF, IL-17, or IL-18.

In some embodiments, the immunomodulatory agent inhibits: inflammasome function; conversion of succinate to fumarate; succinate dehydrogenase (Sdh); IL-17-associated autoimmune inflammation; or frequency of IL-17-producing cells.

In some embodiments, the immunomodulatory agent reduces, suppresses, or down regulates pro-IL-1β, ASC, NLRP3, iNos, 1L6, IL1b, IL18, IFNB1, IL12b, mROS, succinate, iNOS, HIF-1α, Nos2, or Th17 differentiation.

In some embodiments, the immunomodulatory agent suppresses or inhibits secretion or production of IL-1β, IL-6, IL-17, IL-18, IL-12p70, NO, or GM-CSF.

In some embodiments, the immunomodulatory agent: modulates expression of II1b, II18, P2rx7, Casp1, or an inflammasome adapter Pycard (ASC); attenuates hypoxia-induced increase in ROS generation and protects against hypoxia-induced cell death; or regulates succinate levels, mitochondrial respiratory rate, and inflammatory cytokine production during macrophage activation.

In some embodiments, the immunomodulatory agent is formulated as a pharmaceutical composition comprising one or more pharmaceutically acceptable diluents or carriers.

In some embodiments, the disease, disorder, or condition is associated with a lipopolysaccharide (LPS)-mediated immune response and the immunomodulatory agent suppresses a lipopolysaccharide (LPS)-mediated immune response; modulates II1b, II18, P2rx7, Casp1, or an inflammasome adapter Pycard (ASC); impairs LPS-induced upregulation of mtROS; downregulates LPS-induced expression of HIF-1α, or promotes the glutathione-mediated anti-oxidant pathway.

In some embodiments, the disease, disorder, or condition is selected from one or more of the following: adult and juvenile Still disease; asthma; allergy; Alzheimer's disease; age-related macular degeneration; antisynthetase syndrome; autoinflammatory disease; autoimmune disease; autoimmune response; Behcet disease; Blau syndrome; cancer; cardiovascular infarction; chronic infantile neurological cutaneous and articular (CINCA) syndrome; chronic recurrent multifocal osteomyelitis; cinca syndrome; classic autoinflammatory diseases; cryopyrin-associated autoinflammatory syndromes (CAPS); deficiency in IL-1 receptor antagonist (DIRA); diabetes mellitus; Erdheim-Chester syndrome (histiocytosis); extrapulmonary tuberculosis; familial atypical mycobacteriosis; familial cold autoinflammatory syndrome (FCAS); gastric cancer Risk after H. pylori Infection; Guillain-Barré syndrome; Hashimoto's thyroiditis; heart failure; hepatic fibrosis; Huntington's disease; hyper IgD syndrome (HIDS); hypoxia; ischaemia-reperfusion; immunodeficiency 29; inflammation; inflammation by HIV; inflammatory bowel disease (IBD); macrophage activation syndrome (MAS); mycobacteriosis; Miller-Fisher syndrome; Muckle-Wells syndrome (MWS); multiple sclerosis (MS); neonatal-onset multisystem inflammatory disease (NOMID); neuropathic pain; N syndrome; osteoarthritis; osteoporosis; Periodontal Disease; plaque psoriasis; psoriatic arthritis; periodic fever, aphthous stomatitis, pharyngitis, adenitis syndrome (PFAPA); postmyocardial infarction heart failure; psoriasis; recurrent idiopathic pericarditis; recurrent pericarditis; relapsing chondritis; relapsing-remitting multiple sclerosis; rheumatoid arthritis (RA); Sapho Syndrome; Schnitzler syndrome; secondary progressive multiple sclerosis; septic shock; smoldering myeloma; systemic sclerosis; Sweet syndrome; synovitis, acne, pustulosis, hyperostosis, osteitis (SAPHO); systemic juvenile rheumatoid arthritis; familial Mediterranean fever (FMF); pyogenic arthritis; pyoderma gangrenosum, acne (PAPA); TNF receptor-associated periodic syndrome (TRAPS); type 2 diabetes; urate crystal arthritis (gout); urticarial vasculitis; or vitiligo.

In some embodiments, the disease, disorder, or condition is cardiovascular infarction or ischaemia-reperfusion in heart, kidney, or brain and the immunomodulatory agent protects against hypoxia-induced cell death; or the disease, disorder, or condition is psoriasis and the immunomodulatory agent prevents skin edema and reduces inflammation.

In some embodiments, the cardiovascular infarction area is reduced in size.

In some embodiments, reduction in tissue injury is due to reduction in mitochondrial reactive oxygen species (mROS).

In some embodiments, the immunomodulatory agent suppresses immune response or inhibits IL-17-associated autoimmune inflammation.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1I depicts graphs showing itaconate has anti-inflammatory effect on macrophage activation. (FIG. 1A) Volcano plots showing metabolites (left) and transcripts (right) that are differentially expressed between resting and activated BMDM (LPS 100 ng/ml+IFN-V 50 ng/ml, 24 h). The y-axis shows the p value corresponding to fold change (x axis) of each metabolite or transcript (see Methods). Indicated are the specific metabolites and transcripts that show the greatest level of induction in activated cells (top part of plots). (FIG. 1B) relative expression of intracellular (left) and secreted (right) itaconate by BMDM at indicated timepoints after activation (LPS+IFN-γ), as determined by metabolomics profiling. Data shown are mean±standard error of the mean (SEM) of triplicate cultures from one experiment. P values were calculated using two-tailed Student's t-test. (FIG. 1C) Histogram of intracellular NOS2 expression determined by flow cytometry in BMDM pretreated with DI (12 h; 0.25 mM) or untreated (control) and then stimulated with LPS and IFN-γ for 24 h. (FIG. 1D) left bar graph show IL-12 protein levels in the culture supernatants of BMDM pretreated with the indicated doses of DI (12 h) and then activated as in c. Data shown are mean±SEM of triplicate cultures from one of two experiments; middle and right bar graphs show IL-6 and TNF-α secreted by BMDM untreated or pre-treated with DI (12 h; 0.25 mM,) and stimulated with LPS for 24 h. “Med.” indicates resting cells cultured in medium alone. Data shown are mean±SEM of triplicate cultures from one of two experiments. P values were calculated using two-tailed Student's t-test. (FIG. 1E) heatmap of differentially expressed inflammasome-related genes and genes encoding for proinflammatory macrophage markers by unstimulated (Uns), LPS-stimulated (100 ng/ml; 4 h), DI-pretreated (DI+Uns), DI-pretreated and then LPS-stimulated. (DI 0.25 mM 12 h+LPS 100 ng/ml, 4 h) BMDM. (FIG. 1F) bar graphs showing mature IL-1β and IL-18 produced by BMDM that were untreated, or pre-treated for 12 h with the indicated doses of DI and then primed by LPS (100 ng/ml, 4 h) followed by ATP (3 mM, 45 min). Data shown are mean±SEM of triplicate cultures from one of two experiments. P values were calculated using two-tailed Student's t-test. (FIG. 1G) bar graphs show mature IL-1β secreted by BMDM untreated or pre-treated with 0.25 mM DI for 12 h and then primed with LPS as in f, followed by addition of nigericin (Nig. left; 5 mM for 1 h) or monosodium urate crystals (MSU; right; 250 pg/ml for 4 h). Data shown are mean±SEM of triplicate cultures from one of two experiments. (FIG. 1H) Western blot analysis of NLRP3, pro-IL-1β and ASC in cell lysates of BMDM untreated or pretreated with DI (0.25 mM for 12 h), primed with LPS (100 ng/ml for 4 h) and then stimulated with ATP (3 mM for 45 min). α-Tubulin was used as loading control. The blot shown is from one of two representative experiments. (FIG. 1I) Bar graphs show levels of indicated cytokines and nitric oxide (NO) present in the supernatants of untreated or DI-treated BMDM that were infected with live S. typhimurium (see methods) for the indicated periods of time. Data shown are mean±SEM of triplicate cultures per time-point from one of two experiments. P values were calculated using two-tailed Student's t-test.

FIG. 2A-FIG. 2E depicts graphs and images showing itaconate is an inhibitor of Sdh activity. (FIG. 2A) Comparative network showing changes in the magnitude of predicted fluxes between unstimulated macrophages with and without itaconate treatment. Network is obtained using Flux Balance Analysis framework (see Methods) based on gene expression in respective conditions. Blue edges correspond to reactions that have flux only in M0 condition and red edges correspond to reactions that have flux only in M0+itaconate conditions, reactions with equal predicted fluxes are shown in grey. 2PG and PEP stand for 2-phosphoglycerate and phosphoenolpyruvate. (FIG. 2B) Extracellular acidification rate (ECAR) measured in BMDM following treatment with 0.25 mM DI (DI+medium) or vehicle (medium) for 12 h using Seahorse technology (see Methods). Data shown are mean±SEM of 10-15 replicates per condition from one of two experiments. (FIG. 2C) Chemical structure of succinate, malonate and itaconate (FIG. 2D) Bar graphs show activity of purified Sdh (from a macrophage cell line) in the presence of indicated does of itaconate (calculated relative to control SDH activity in the absence of itaconate; see methods). Data shown are mean±SEM of two independent experiments performed in duplicates. (FIG. 2E) bar graphs show mature IL-1β produced by BMDM untreated or pre-treated for 12 h with the indicated doses of dimethyl malonate (DM), then primed by LPS (100 ng/ml, 4 h) and stimulated with ATP (3 mM, 45 min). Data shown are mean±SEM of triplicate cultures from one of two experiments. P values were calculated using two-tailed Student's t-test.

FIG. 3A-FIG. 3F depicts graphs and a schematic showing endogenous itaconate controls TCA cycle remodeling and succinate levels. (FIG. 3A) relative expression of intracellular (FIG. 3A; left) and secreted (right) itaconate by WT and Irg^{-/-} BMDM at indicated timepoints after activation with LPS (20 ng/ml) and IFN-γ (50 ng/ml) as determined by metabolomics profiling of respectively, cell extracts and culture supernatants. Data shown are mean±SEM of triplicate cultures per timepoint from one experiment (FIG. 3B) Relative expression of succinate, fumarate and malate in cell extracts of BMDM activated as in a and determined as in a. Data shown are mean±SEM of triplicate samples per time-point from one experiment. (FIG. 3C) scheme showing how itaconate regulates TCA flow in LPS-activated macrophages by inhibiting Sdh as revealed by changes in metabolites in Irg1^{-/-} BMDM observed in b. (FIG. 3D) Basal oxygen consumption rate by resting (left) and LPS-activated BMDM (right; 100 ng/ml, 24 h) from WT and Irg1^{-/-} mice, as measured using Seahorse technology. Data shown are mean±SEM of 13 to 18 technical replicates from one experiment. P values were calculated using two-tailed Student's t-test. (FIG. 3E) bar graphs show IL-12 protein levels in supernatants of WT and Irg1^{-/-} BMDM stimulated with LPS (20 ng/ml) and IFN-γ (50 ng/ml) for 24 h. Data shown are mean±SEM of triplicate cultures from one experiment. P values were calculated using two-tailed Student's t-test. (FIG. 3F) bar graphs show mature IL-1β secreted by WT and Irg1^{-/-} BMDM primed with LPS (100 ng/ml for 4 h) and stimulated with ATP (3 mM, 45 min). Data shown are mean±SEM of triplicate cultures from one of two experiments. P values were calculated using two-tailed Student's t-test.

FIG. 4A-FIG. 4G depicts graphs and an image showing that itaconate acts as an inhibitor of Sdh in vivo and modulates ROS mediated injury in an ischemia model. (FIG. 4A) Representative Evans Blue and TTC stained sections of hearts subjected to ischemia-reperfusion injury, following pretreatment with DI or saline as control. (FIG. 4B, FIG. 4C) Quantitation of area-at-risk (AAR) and infarct area (IA) as % of AAR (FIG. 4B) and left ventricular (LV) myocardium (FIG. 4C) from mice treated as in a (Saline, n=8; DI, n=7). P values were calculated using two-tailed Student's t-test. (FIG. 4D) Percent change in ROS generation (over respective normoxic controls) in neonatal rat cardiac myocytes (NRCMs) subjected to hypoxia for 24 h in the presence of DI or diluent. P values were calculated using post-hoc test after one-way ANOVA (n=4 per condition). (FIG. 4E) percentage of cell death in NRCMs treated as in d. P values were calculated using post-hoc test after one-way ANOVA (n=8 per condition). (FIG. 4F) Histograms of mitochondrial ROS (mROS) expression detected by mitoSox reagent via flow cytometry, in BMDM pretreated or not with DI (0.25 mM for 12 h) then stimulated with LPS 100 ng/ml for 3 h. Medium indicates untreated non-stimulated cells. (FIG. 4G) Fold change in mROS mean fluorescence intensity (relative to Medium) measured in f. Data shown are mean±SEM of three independent experiments performed in duplicates. P value was calculated using two-tailed Student's t-test.

FIG. 5 depicts a graph showing dose-dependent effect of itaconate on cell viability. BMDM were pre-treated with the indicated doses of dimethyl itaconate (DI) for 12 h and subsequently stimulated (LPS, 100 ng/ml) or not (Med.) for 4 h. Live cells were determined via flow cytometry using a live/dead cell fluorescent dye. Shown are representative data from three independent experiments.

FIG. 6 depicts a chart showing pathways transcriptionally regulated by DI treatment of macrophages. Gene Set Enrichment Analysis shows a list of differentially regulated pathways—proinflammatory pathways are downregulated, while Phase II conjugation, glutathione conjugation and biological oxidations are upregulated upon itaconate addition.

FIG. 7A-FIG. 7B depicts graphs showing that DI marginally affects LPS+ATP-induced cytotoxicity and inhibits mature IL-1β in response to AIM2-inflammasome activation. FIG. 7A shows a bar graph of BMDM pre-treated or not with 0.25 mM DI for 12 h, subsequently primed with LPS (100 ng/ml for 4 h), and then stimulated or not with ATP (3 mM, 45 min). Live cells were determined via flow cytometry using a live/dead cell fluorescent dye. Medium indicates untreated and non-stimulated cells, whereas DI indicates DI-treated non-stimulated cells. Bar graphs show mean±s.e.m. of pooled data from two independent experiments (n=4 per condition). FIG. 7B is a bar graph showing mature IL-1β produced by BMDM that were pre-treated for 12 h with the indicated doses of DI and then transfected with either poly dA:dT mixed with Xfect polymer or with polymer alone followed by 5 h incubation. Med, indicates cells cultured in medium alone. Bar graphs show mean±s.e.m. of triplicate cultures from a single experiment.

FIG. 8 depicts graphs showing that itaconate treatment moderately affects TNF-α production but has no bactericidal activity against S. typhimurium in BMDMs. FIG. 8A bar graph shows TNF-α levels present in the supernatants of untreated or DI-treated BMDM that were subsequently infected with live S. typhimurium (see Methods) for the indicated periods of time. FIG. 8B bar graph shows numbers of intracellular bacteria (see Methods) determined at the indicated time-points after infection of untreated or DI-treated BMDM with S. typhimurium. Data in a and b are mean±s.e.m. of triplicate cultures per timepoint from one of two experiments. P values were calculated using two-tailed Student's t-test.

FIG. 9 depicts a graph showing that murine macrophages upregulate Irg1 expression in response to viral infection. Irg1 expression in macrophages infected with active or inactive Sendai virus as extracted from public data from GSE2935 (PMID: 16208318).

FIG. 10 depicts a schematic showing the complete Flux balance Analysis Network. The complete comparative network of flux changes that occurred in resting WT BMDM in response to itaconate treatment. Blue edges indicate decreased fluxes, in red are increased fluxes and in grey fluxes unresponsive to DI treatment. This network includes the fluxes that are highlighted in FIG. 2A.

FIG. 11A-FIG. 11B depicts graphs showing that itaconate and malonate inhibit activity of macrophage-derived Sdh. (FIG. 11A) Bar graph shows activity of BMDM-derived Sdh in the presence of the indicated dose of itaconate. (FIG. 11B) Bar graph shows activity of BV2 cell-derived Sdh in the presence of the indicated doses of malonate. Data shown are representative of two experiments.

FIG. 12A-FIG. 12C depicts a schematic, immunoblot and graph showing the generation and validation of Irg1^{-/-} mice. (FIG. 12A) Scheme of Irg1 locus with targeting cassette. Exons are noted in grey and target location for insertion is noted with dashed lines. (FIG. 12B) Irg1 gene deletion was verified by PCR by the presence of 501 bp band in the mutant allele, whereas WT Irg1 manifests as a 436 bp band. (FIG. 12C) 3′ RNA-seq Irg1 gene coverage plot for WT and Irg^{-/-} macrophages after 24 h stimulation with LPS and IFN-γ. Part of Irg1 mRNA is expressed in Irg1^{-/-} cells, but transcription downstream of the exon 4 is prevented.

FIG. 13 depicts a heatmap showing the transcriptional signatures of activated Irg1^{-/-} and DI-treated BMDM are inversely related. Gene set enrichment analysis shows that genes upregulated in Irg1^{-/-} BMDM were downregulated under conditions of itaconate treatment of WT BMDM (for top 200 differentially upregulated genes, see gene lists in Supplementary tables).

FIG. 14A-FIG. 14B depicts immunoblots showing that itaconate regulates LPS-induced expression of HIF-1α. FIG. 14A is a Western blot analysis of HIF-1 in lysates from WT and Irg^{-/-} BMDM stimulated or not with LPS (100 ng/ml for 4 h). FIG. 14B is a Western blot analysis of HIF-1 in lysates from WT BMDM pretreated or not with DI (0.25 mM, 12 h) and then stimulated with LPS (100 ng/ml for 24 h). α-tubulin was used as loading control. Each blot is representative of two independent experiments.

FIG. 15 shows a scheme of the interdisciplinary approach adopted to characterize metabolic rewiring in macrophage activation shows three main stages: (1) high-throughput profiling of the system; (2) computational analysis and hypothesis generation; (3) using classical experimental immunology techniques to validate emerging hypothesis in vitro and in vivo.

FIG. 16 shows a schematic representation of the global metabolic rewiring during macrophage activation by LPS: glycolytic flux increases dramatically immediately after activation, and TCA cycle becomes dysfunctional due to break point in metabolic flux at the isocitrate dehydrogenase (Idh1) that redirects the metabolic flow toward itaconate and fatty-acid production. It is currently hypothesized that second TCA cycle breakpoint leading to succinate accumulation is controlled by itaconate via inhibition of Sdh due to its structural similarity with succinate, substrate of Sdh.

FIG. 17A shows a vulcano plot of intracellular metabolites regulated between M1- and M2-polarized macrophages: on the right hand side are metabolites that are most specific to M1 macrophages, most notable ones are arginine and itaconic acid.

FIG. 17B shows chemical structures of succinate (Sdh substrate), and malonate and itaconate (Sdh inhibitors).

FIG. 18A-FIG. 18D shows itaconate has distinct anti-inflammatory effect in LPS and LPS+ATP stimulated macrophages: (FIG. 18A) Heatmap showing a subset of inflammatory marker genes based on RNA-seq profiling of macrophages stimulated with LPS in the presence and absence of itaconate; (FIG. 18B) FACS analysis of intracellular expression of iNOS protein after LPS stimulation in the presence and absence of itaconate shows that protein levels are decreased similarly to transcript levels; (FIG. 18C, FIG. 18D) secretion of II1b and II18 cytokines in the media after inflammasome stimulation (LPS+ATP) is dose dependently inhibited by addition of itaconate.

FIG. 19 A shows a Western blot analysis in stimulated macrophages shows decreased expression of proll1b in the presence of itaconate.

FIG. 19B shows a FACS for MitoSox probe showing that itaconate inhibits mitochondrial ROS production by LPS stimulation.

FIG. 20 is a bar graph showing the metabolic profiling of itaconate shows that Irg1−/− cells completely lack itaconate production upon LPS.

FIG. 21A-FIG. 21C are a series of bar graphs and an illustration showing the metabolomic profiling of succinate (FIG. 21A) and fumarate (FIG. 21B) levels in WT and Irg1−/− macrophages upon activation with LPS at 0, 10 and 24 hours shows succinate accumulation is itaconate dependent and consistent with (FIG. 21C) the inhibitory effects on Sdh.

FIG. 22A is a representative image of the heart slices from ischaemia-reperfusion experiment. Blue shows are not at risk, red and white combined are at risk with white being infarct area and red—healthy, unaffected cells. Infarct area is distinctly bigger in the absence of complex II inhibitors.

FIG. 22B is a whisker plot showing the quantification of the infarct sizes.

FIG. 23 is a graph showing continuous OCR measurements performed in Seahorse Analyzer follow mitochondrial oxygen consumption upon consecutive addition of various ETC inhibitors and substrates

FIG. 24 is an image showing the most regulated subnetwork identified by computational analysis for comparison between M1 and M2 polarized macrophages. (Global murine metabolic network consists of more than 2000 enzymes and metabolites measured) The most regulated metabolic subnetwork encompasses 7 distinct modules, highlighted by distinct background shading. Three major novel features of macrophage polarization identified in our previous work are highlighted with dotted line squares—green for M1-specific module and red for M2. Round nodes represent metabolites within core regulatory network. Enzymes are represented by square nodes.

FIG. 25A-FIG. 25F is a series of graphs showing that itaconate, both in its acid and ester form, can suppress Th17 differentiation in a structure-specific manner. Purified naïve CD4 T cells (from C56BL/6 mice) were differentiated into Th17 cells under the typical Th17 polarizing cytokine conditions (IL-6, IL-1, IL-23, TGFβ, anti-IFNγ, anti-IL-4) in presence or absence of DI (dimethyl itaconate; FIG. 25A-FIG. 25C), Itaconic or related acids (FIG. 25D-FIG. 25F). FIG. 25A is a plot showing DI has a moderate dose-dependent cytotoxic effect. Plots show the frequency of live CD4 T cells as determined by flow cytometry on day 4 after Th17 polarization in presence of the indicated doses of DI. FIG. 25B is a flow cytometry plot and bar graph showing DI dose-dependently inhibits the frequency of IL-17-producing cells. (left) Representative flow cytometry plot showing how the frequency of IL-17+CD4 T cells was determined (top left+ right quadrat). (right) Bar graph shows the frequency IL-17+ cells determined under different DI doses. FIG. 25C is a series of plots showing DI inhibits also the secretion of IL-17 (left) and GM-CSF (right) in the supernatant of CD4 T cells. On day 4 cells were collected, washed, and re-stimulated in equal numbers for each condition with anti-CD3 and anti-CD28. After 24 h supernatants were collected and assayed for IL-17 and GM-CSF by ELISA. FIG. 25D is a series of bar graphs showing itaconic acid is not cytotoxic under Th17 conditions and dose-dependently inhibits Th17 differentiation. (Left) bar graph shows the frequency of live CD4 T cells determined as in A; (right) bar graph shows the frequency of IL-17+ cells determined as in FIG. 25B. FIG. 25E are chemical structures of itaconic acid and structurally similar dicarboxylic acids. FIG. 25F is a series of bar graphs showing inhibition of Th17 by itaconic acid is structure specific; (Left) bar graph shows the frequency of live CD4 T cells determined as in A on day 3 post polarization. (right) bar graph shows the frequency of IL-17-producing cells determined as in A. All acids were used at 5 mM.

FIG. 26A-FIG. 26B is a series of graphs and images showing that in vivo administration of itaconate inhibit IL-17-associated autoimmune inflammation in vivo. FIG. 26A is a plot showing DI-treatment diminishes in vivo systemic IL-17 production. Circulating serum IL-17 levels were determined by ELISA 48 h after anti-CD3 induced inflammation in mice treated or not with the indicated doses of DI. Briefly, mice received intraperitoneal (ip) injections of 4 mg (n=10) or 20 mg (n=5) DI or vehicle (PBS; shown as 0; n=10) 3 h prior (−3 h) to an anti-CD3 injection (given at 0 h ip) and every 20 h thereafter until mice were analyzed at 48 h. FIG. 26B is a series of histology images showing DI administration in vivo limits IL-17 associated pathology in imiquimod (IMQ)-induced psoriasis model. IMQ cream (5%) was applied on mouse ear skin for 7 days and skin pathology was assessed on day 8. Mice received 20 mg DI ip. (DI+IMQ) or vehicle ip (PBS; IMQ) was given one day prior to IMQ application and every day thereafter until day 7. DI group: received 20 mg DI as above but no IMQ; naïve mice received vehicle as above but no IMQ. Images shows hematoxylin and eosin stains of mouse ear skin sections of the groups described above. Administration of DI prevents dermal edema that occurs during IMQ-induced skin inflammation.

### DETAILED DESCRIPTION OF THE INVENTION

The present disclosure is based, at least in part, on the discovery that itaconate is an endogenous metabolic regulator of the inflammatory functions of succinate.

As shown herein, succinate regulation arises during the process of metabolic remodeling via the actions of itaconate—one of the most highly induced metabolites in activated macrophages. It is also shown that itaconate regulates the inflammatory activity of succinate via its effect on succinate dehydrogenase (Sdh), by inhibiting conversion of succinate to fumarate. Addition of exogenous itaconate resulted in anti-inflammatory effects in vitro and in vivo using models of macrophage activation and ischemia-reperfusion injury. Furthermore, using newly generated Irgf^{-/-} mice, which lack the ability to produce itaconate, it is shown that endogenous itaconate regulates succinate levels, mitochondrial respiratory rate, and inflammatory cytokine production during macrophage activation. These studies highlight itaconate as a major physiological regulator of the global metabolic rewiring in activated macrophages.

Stimulation-induced metabolic rewiring in immune cells can bear a dual purpose: it reflects altered needs for energy and material but also plays a regulatory and signaling function. Studies have focused on dissecting regulatory capacity of metabolic changes associated with macrophage activation. Previously it was demonstrated that succinate (TCA cycle metabolite) acts as a key proinflammatory signal, and this work was followed by intense studies of succinate's functional roles in macrophages. Yet, regulatory mechanisms that control succinate accumulation and utilization still remain unknown. As described herein, a systems biology approach was used—the global metabolic architecture of macrophage activation was dissected and showed that upon LPS stimulation, the TCA cycle becomes disrupted by inactivating isocitrate dehydrogenase (Idh1) which frees citrate to participate in the production of acetyl-CoA, an important fatty acid synthesis precursor, and in the intracellular production of itaconic acid, a dicarboxilyc acid metabolite structurally very similar to succinate. Here, an unexpected role for itaconate as an endogenous, negative regulator of succinate's inflammatory function is explored.

Understanding that metabolites can play regulatory roles has revolutionized the way of thinking about cellular metabolism. Targeting metabolic circuits has shown to be an effective approach to modify cellular behavior, first in the context of cancer biology, yielding a number of metabolism modulating drugs in various clinical stages. The field of immunology quickly followed to discover critical importance of the metabolic rewiring for the immune responses, both innate and adaptive. In spite of now well-recognized importance of the metabolic control of immune system, detailed fundamental understanding of many mechanisms regulating metabolic rewiring is lacking. One prominent example of metabolites playing a regulatory role is a pro-inflammatory effect of succinate that was originally described in the context of macrophage activation, and later expanded to other cellular contexts such as ischaemia-reperfusion injury in heart, kidney and brain. Since then, a number of studies have detailed the mechanisms via which succinate exerts its effects on the immune system yet leaving unanswered the critical question of how succinate itself is regulated. The data suggests that another metabolite produced by macrophages upon LPS stimulation—itaconate—is a major natural regulator of the inflammatory function of succinate. This observation advances the understanding of the metabolic rewiring during macrophage activation. Importantly, itaconate is produced in response to inflammatory stimuli in both human and mouse macrophages, suggesting that itaconate could be clinically relevant natural compound with immunomodulatory effects. Studies to detail the mechanism of itaconate's anti-inflammatory action both in vivo and in vitro are described herein.

Apart from delineating unique importance of itaconate as a regulator of succinate's inflammatory action, the experiments described in Examples 2-5 also presents a unique opportunity to develop an integrated view of metabolic rewiring during immune response. One of the major difficulties in studying metabolism stems from its complex regulatory structure: in addition to relative levels of metabolites, metabolic rewiring is also directly controlled by enzymes, whose levels are determined via transcriptional, epigenetic, and other regulatory mechanisms. In this regard, macrophages serve as an excellent model for studying metabolic control of the immune responses. Apart from metabolic changes, the process of macrophage activation involves a coordinated rewiring of macrophage physiology at multiple regulatory levels that are already characterized to a degree: 1) signaling pathways driving cellular response to distinct in vitro stimulations (LPS, LPS+ATP, etc.) have been well described; and 2) large collections of transcriptional and epigenetic data for macrophage activation are publicly available through databases such as GEO. Finally, macrophages provide tractable system for translating between in vitro and in vivo phenotypes (for instance, connecting inflammasome activation, metabolism and Salmonella infection). Overall, the experiments as described herein provide a unique opportunity to dissect metabolic rewiring during macrophage activation and characterize critical immunomodulatory role of itaconate.

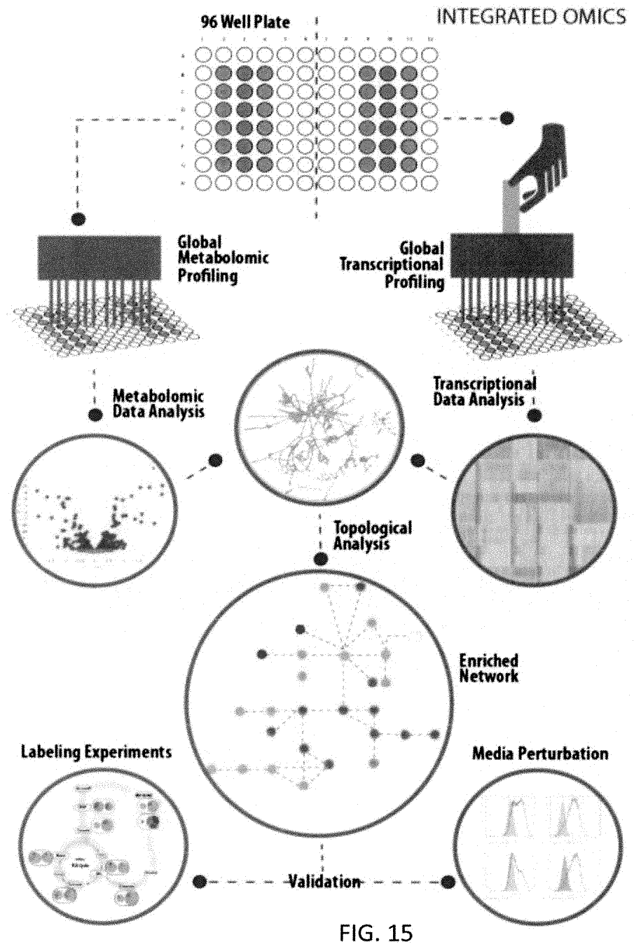

The complex nature of regulatory mechanisms underlying metabolic rewiring warrants integrated systems level approach, which includes (1) classical immunological experimental approaches, but also (2) high throughput data generation, and (3) analysis focused on the formulating the hypotheses that are experimentally testable in the context of immune response (see e.g., FIG. 15). This delicate combination requires cross-disciplinary approach, as described herein. An important feature of the approach described herein is the development and use of novel computational approaches with the goal of gaining specific functional insights that can be validated directly in the experiments as opposed to providing generic “spaghetti network” description of the data.

As a result of these efforts, a state-of-the-art high-throughput transcriptional-metabolic profiling and analysis pipeline is described herein, which was applied to characterize global rewiring during murine macrophage polarization to M1 and M2 states in vitro.

As described herein, itaconate becomes highly upregulated both as an intracellular and extracellular metabolite in LPS-activated macrophages. It was then asked whether secreted itaconate might act in an autocrine manner on macrophages themselves. In view of itaconate's dramatic suppressive effects on activated macrophages, determination of its potential paracrine effect on other cells types was sought. One of the cells types that activated macrophages often encounter in vivo in secondary lymphoid organs and/or in affected tissues during numerous diseases in humans and rodents, are CD4 T cells. Activated myeloid cells, like macrophages and dendritic cells, can drive and/or promote the differentiation of naïve CD4 T cells into distinct T helper (Th) subsets with effector functions that are commonly defined by the production of a specific array of cytokines. Of these subsets, the so-called Th17 subset (Th17 cells) and its signature cytokines including IL-17, GM-CSF, IL-22 and others, have been most frequently associated with autoimmune pathology in diseases as diverse as inflammatory bowel disease, psoriasis, multiple sclerosis, rheumatoid arthritis and others. Importantly, there is currently no cure for these diseases and the available treatments have partial and often only short-term success.

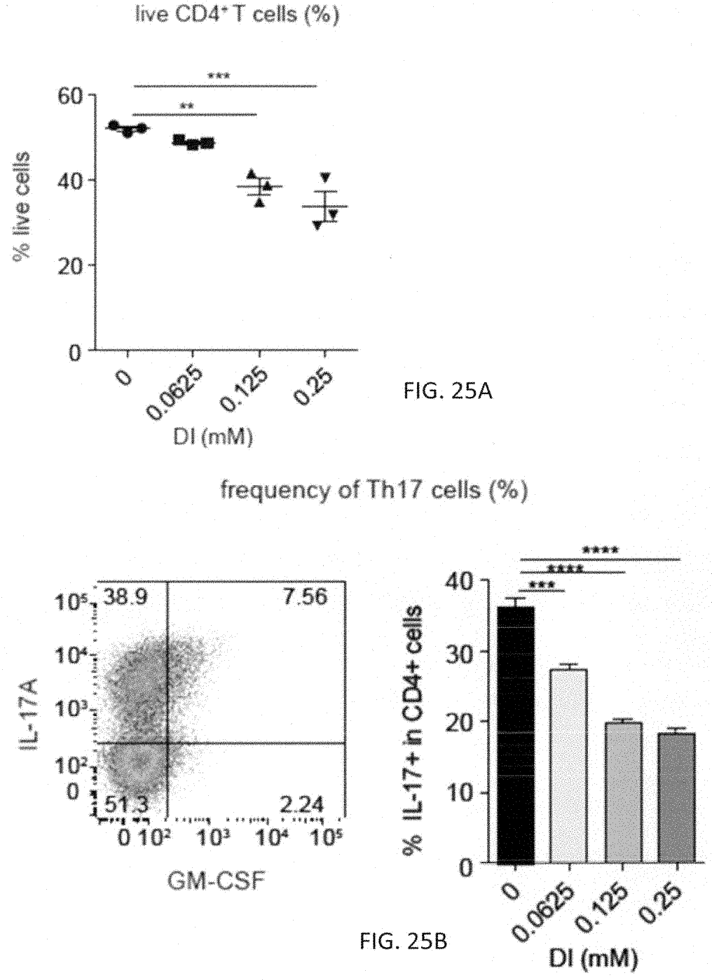

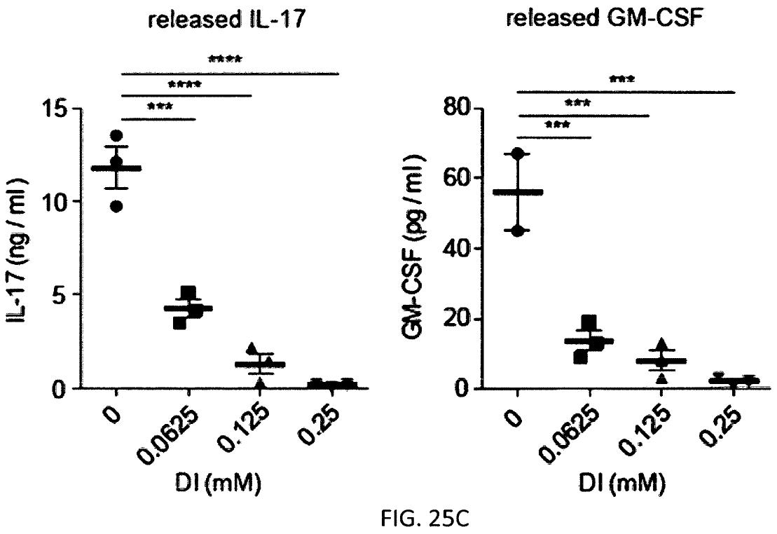

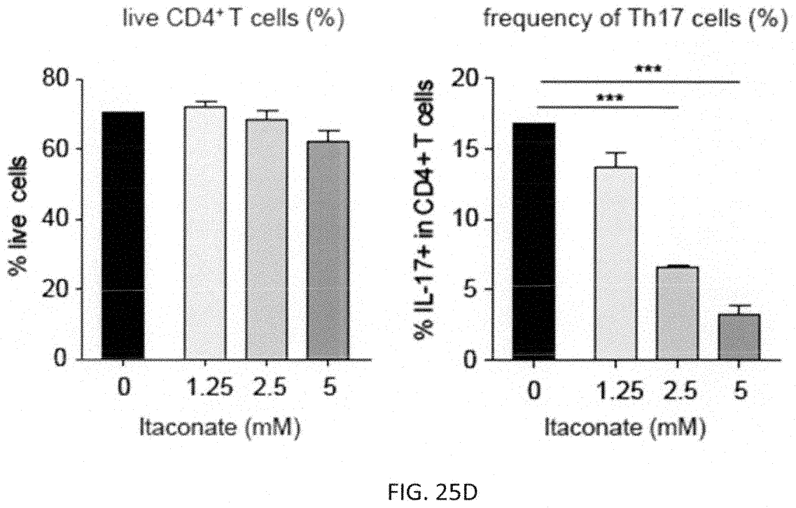

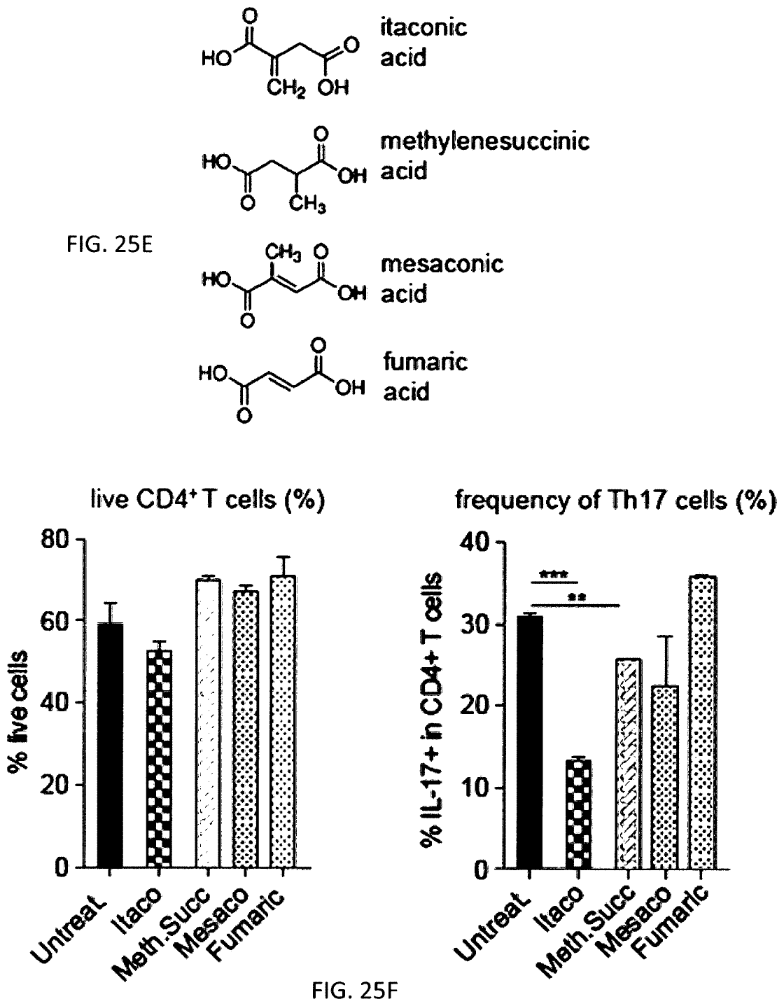

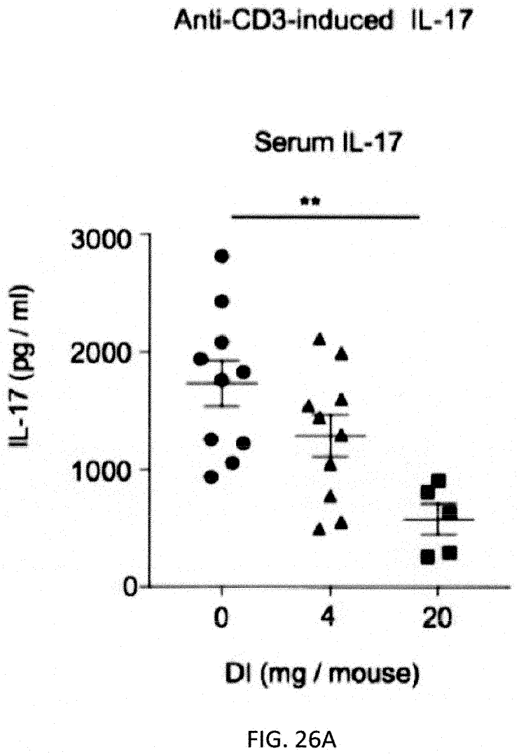

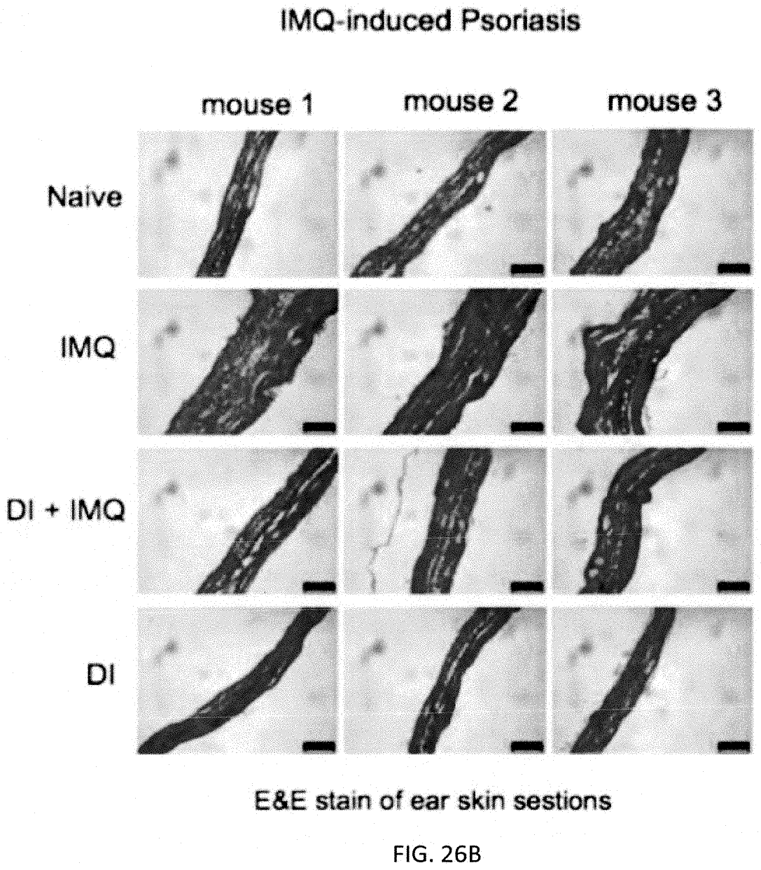

For these reasons investigation of the effect of itaconate on the differentiation of Th17 cells in vitro (FIG. 25) and Th17 associated autoimmune inflammation in vivo (FIG. 26) was studied. It was shown that itaconate suppresses Th17 differentiation, GM-CSF production, and inhibits IL-17-associated inflammation in vivo.

Immunomodulatory Agents

Examples of immunomodulatory agents are described herein.

Immunomodulatory agents can be, for example, modulators of immune activity. Examples of such agents can be small organic molecules that regulate pathways. For example, the immunomodulatory agents can downregulate proinflammatory pathways and upregulate Phase II conjugation, glutathione conjugation, and biological oxidations. As another example, an immunomodulatory agent can modulate the production of cytokines and gene expression.

An immunomodulatory agent can be an itaconate, a malonate, or a derivative thereof. An itaconate, malonate, or a derivative thereof can have a formula of:

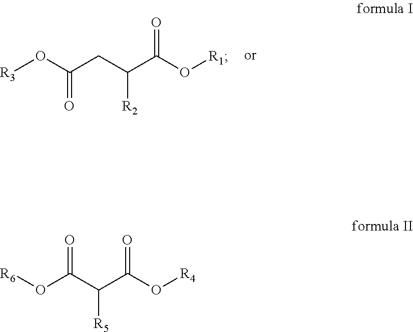

R₁ is hydrogen, unsubstituted or substituted alkyl; unsubstituted or substituted alkenes; or unsubstituted or substituted alkynes;

R₂ is hydrogen, unsubstituted or substituted alkyl; unsubstituted or substituted alkenes; or unsubstituted or substituted alkynes;

R₃ is hydrogen, unsubstituted or substituted alkyl; unsubstituted or substituted alkenes; or unsubstituted or substituted alkynes;

R₄ is hydrogen, unsubstituted or substituted alkyl; unsubstituted or substituted alkenes; or unsubstituted or substituted alkynes;

R₅ is hydrogen, unsubstituted or substituted alkyl; unsubstituted or substituted alkenes; or unsubstituted or substituted alkynes; and

R₆ is hydrogen, unsubstituted or substituted alkyl; unsubstituted or substituted alkenes; or unsubstituted or substituted alkynes;

wherein

R₁, R₂, R₃, R₄, and R₅ can be optionally substituted with one or more groups independently selected from the group consisting of hydroxyl; hydroxyl; amine; C₁₋₁₀carboxylic acid; C₁₋₁₀carboxyl, straight chain or branched C₁₋₁₀alkyl, optionally containing unsaturation; a C₂₋₆ cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl, wherein

the unsubstituted phenyl ring or substituted phenyl ring can be optionally substituted with one or more groups independently selected from the group consisting of hydroxyl; hydroxyl; amine; C₁₋₁₀carboxylic acid; C₁₋₁₀carboxyl, straight chain or branched C₁₋₁₀alkyl, optionally containing unsaturation; straight chain or branched C₁₋₁₀alkyl amine, optionally containing unsaturation; a C₂₋₆ cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and

the unsubstituted heterocyclyl or substituted heterocyclyl can be optionally substituted with one or more groups independently selected from the group consisting of hydroxyl; C₁₋₁₀alkyl hydroxyl; amine; C₁₋₁₀carboxylic acid; C₁₋₁₀carboxyl, straight chain or branched C₁₋₁₀alkyl, optionally containing unsaturation; straight chain or branched C₁₋₁₀alkyl amine, optionally containing unsaturation; a C₂₋₆ cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms.

In some embodiments, the immunomodulatory agent is

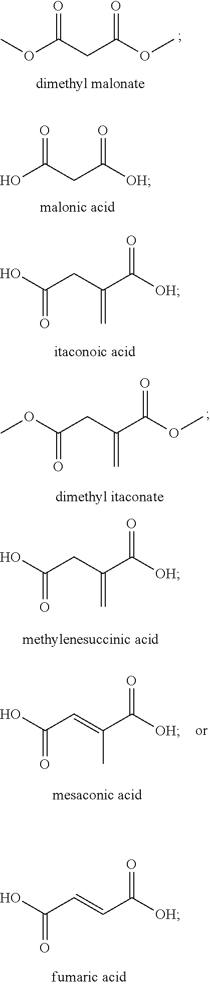

or a derivative, or a pharmaceutically acceptable salt thereof, including all tautomers and stereoisomers.

In some embodiments, the immunomodulatory agent is not dimethyl fumaric acid.

The term “imine” or “imino”, as used herein, unless otherwise indicated, includes a functional group or chemical compound containing a carbon-nitrogen double bond. The expression “imino compound”, as used herein, unless otherwise indicated, refers to a compound that includes an “imine” or an “imino” group as defined herein.

The term “hydroxyl”, as used herein, unless otherwise indicated, includes —OH.

The terms “halogen” and “halo”, as used herein, unless otherwise indicated, include a chlorine, chloro, Cl, fluorine, fluoro, F; bromine, bromo, Br; or iodine, iodo, or I.

The term “aryl”, as used herein, unless otherwise indicated, include a carbocyclic aromatic group. Examples of aryl groups include, but are not limited to, phenyl, benzyl, naphthyl, or anthracenyl.

The terms “amine” and “amino”, as used herein, unless otherwise indicated, include a functional group that contains a nitrogen atom with a lone pair of electrons and wherein one or more hydrogen atoms have been replaced by a substituent such as, but not limited to, an alkyl group or an aryl group.

The term “alkyl”, as used herein, unless otherwise indicated, includes saturated monovalent hydrocarbon radicals having straight or branched moieties, such as but not limited to, methyl, ethyl, propyl, butyl, pentyl, hexyl, octyl groups, etc. Representative straight-chain lower alkyl groups include, but are not limited to, -methyl, -ethyl, -n-propyl, -n-butyl, -n-pentyl, -n-hexyl, -n-heptyl and -n-octyl; while branched lower alkyl groups include, but are not limited to, -isopropyl, -sec-butyl, -isobutyl, -tert-butyl, -isopentyl, 2-methylbutyl, 2-methylpentyl, 3-methylpentyl, 2,2-dimethylbutyl, 2,3-dimethylbutyl, 2,2-dimethylpentyl, 2,3-dimethylpentyl, 3,3-dimethylpentyl, 2,3,4-trimethylpentyl, 3-methylhexyl, 2,2-dimethylhexyl, 2,4-dimethylhexyl, 2,5-dimethylhexyl, 3,5-dimethylhexyl, 2,4-dimethylpentyl, 2-methylheptyl, 3-methylheptyl, unsaturated C₁-C₈ alkyls include, but are not limited to, -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, 1-hexyl, 2-hexyl, 3-hexyl, -acetylenyl, -propynyl, -1-butynyl, -2-butynyl, -1-pentynyl, -2-pentynyl, or -3-methyl-1 butynyl. An alkyl can be saturated, partially saturated, or unsaturated.

The term “carboxyl”, as used herein, unless otherwise indicated, includes a functional group consisting of a carbon atom double bonded to an oxygen atom and single bonded to a hydroxyl group (—COOH).

The term “alkenyl”, as used herein, unless otherwise indicated, includes alkyl moieties having at least one carbon-carbon double bond wherein alkyl is as defined above and including E and Z isomers of said alkenyl moiety. An alkenyl can be partially saturated or unsaturated.

The term “alkynyl”, as used herein, unless otherwise indicated, includes alkyl moieties having at least one carbon-carbon triple bond wherein alkyl is as defined above. An alkynyl can be partially saturated or unsaturated.

The term “acyl”, as used herein, unless otherwise indicated, includes a functional group derived from an aliphatic carboxylic acid, by removal of the hydroxyl (—OH) group.

The term “alkoxyl”, as used herein, unless otherwise indicated, includes O-alkyl groups wherein alkyl is as defined above and O represents oxygen. Representative alkoxyl groups include, but are not limited to, —O-methyl, —O-ethyl, —O-n-propyl, —O-n-butyl, —O-n-pentyl, —O-n-hexyl, —O-n-heptyl, —O-n-octyl, —O-isopropyl, —O-sec-butyl, —O-isobutyl, —O-tert-butyl, —O-isopentyl, —O-2-methylbutyl, —O-2-methylpentyl, —O-3-methylpentyl, —O-2,2-dimethylbutyl, —O-2,3-dimethylbutyl, —O-2,2-dimethylpentyl, —O-2,3-dimethylpentyl, —O-3,3-dimethylpentyl, —O-2,3,4-trimethylpentyl, —O-3-methylhexyl, —O-2,2-dimethylhexyl, —O-2,4-dimethylhexyl, —O-2,5-dimethylhexyl, —O-3,5-dimethylhexyl, —O-2,4dimethylpentyl, —O-2-methylheptyl, —O-3-methylheptyl, —O-vinyl, —O-allyl, —O-1-butenyl, —O-2-butenyl, —O-isobutylenyl, —O-1-pentenyl, —O-2-pentenyl, —O-3-methyl-1-butenyl, —O-2-methyl-2-butenyl, —O-2,3-dimethyl-2-butenyl, —O-1-hexyl, —O-2-hexyl, —O-3-hexyl, —O-acetylenyl, —O-propynyl, —O-1-butynyl, —O-2-butynyl, —O-1-pentynyl, —O-2-pentynyl and —O-3-methyl-1-butynyl, —O-cyclopropyl, —O-cyclobutyl, —O-cyclopentyl, —O-cyclohexyl, —O-cycloheptyl, —O— cyclooctyl, —O-cyclononyl and —O-cyclodecyl, —O—CH₂-cyclopropyl, —O—CH₂-cyclobutyl, —O—CH₂-cyclopentyl, —O—CH₂-cyclohexyl, —O—CH₂-cycloheptyl, —O—CH₂-cyclooctyl, —O—CH₂-cyclononyl, —O—CH₂-cyclodecyl, —O—(CH₂)₂-cyclopropyl, —O—(CH₂)₂-cyclobutyl, —O—(CH₂)₂-cyclopentyl, —O—(CH₂)₂-cyclohexyl, —O—(CH₂)₂-cycloheptyl, —O—(CH₂)₂-cyclooctyl, —O—(CH₂)₂-cyclononyl, or —O—(CH₂)₂-cyclodecyl. An alkoxyl can be saturated, partially saturated, or unsaturated.

The term “cycloalkyl”, as used herein, unless otherwise indicated, includes a non-aromatic, saturated, partially saturated, or unsaturated, monocyclic or fused, spiro or unfused bicyclic or tricyclic hydrocarbon referred to herein containing a total of from 3 to 10 carbon atoms, preferably 3 to 8 ring carbon atoms. Examples of cycloalkyls include, but are not limited to, C₃-C₈ cycloalkyl groups include, but are not limited to, -cyclopropyl, -cyclobutyl, -cyclopentyl, -cyclopentadienyl, -cyclohexyl, -cyclohexenyl, -1,3-cyclohexadienyl, -1,4-cyclohexadienyl, -cycloheptyl, -1,3-cycloheptadienyl, -1,3,5-cycloheptatrienyl, -cyclooctyl, and -cyclooctadienyl.

The term “cycloalkyl” also includes -lower alkyl-cycloalkyl, wherein lower alkyl and cycloalkyl are as defined herein. Examples of -lower alkyl-cycloalkyl groups include, but are not limited to, —CH₂-cyclopropyl, —CH₂-cyclobutyl, —CH₂-cyclopentyl, —CH₂-cyclopentadienyl, —CH₂-cyclohexyl, —CH₂-cycloheptyl, or —CH₂-cyclooctyl.

The term “heterocyclic”, as used herein, unless otherwise indicated, includes an aromatic or non-aromatic cycloalkyl in which one to four of the ring carbon atoms are independently replaced with a heteroatom from the group consisting of O, S and N. Representative examples of a heterocycle include, but are not limited to, benzofuranyl, benzothiophene, indolyl, benzopyrazolyl, coumarinyl, isoquinolinyl, pyrrolyl, pyrrolidinyl, thiophenyl, furanyl, thiazolyl, imidazolyl, pyrazolyl, triazolyl, quinolinyl, pyrimidinyl, pyridinyl, pyridonyl, pyrazinyl, pyridazinyl, isothiazolyl, isoxazolyl, (1,4)-dioxane, (1,3)-dioxolane, 4,5-dihydro-1H-imidazolyl, or tetrazolyl. Heterocycles can be substituted or unsubstituted. Heterocycles can also be bonded at any ring atom (i.e., at any carbon atom or heteroatom of the heterocyclic ring). A heterocyclic can be saturated, partially saturated, or unsaturated.

The term “cyano”, as used herein, unless otherwise indicated, includes a —CN group.

The term “alcohol”, as used herein, unless otherwise indicated, includes a compound in which the hydroxyl functional group (—OH) is bound to a carbon atom. In particular, this carbon center should be saturated, having single bonds to three other atoms.

The term “solvate” is intended to mean a solvate form of a specified compound that retains the effectiveness of such compound. Examples of solvates include compounds of the invention in combination with, for example: water, isopropanol, ethanol, methanol, dimethylsulfoxide (DMSO), ethyl acetate, acetic acid, or ethanolamine.

The term “mmol”, as used herein, is intended to mean millimole. The term “equiv”, as used herein, is intended to mean equivalent. The term “mL”, as used herein, is intended to mean milliliter. The term “g”, as used herein, is intended to mean gram. The term “kg”, as used herein, is intended to mean kilogram. The term “μg”, as used herein, is intended to mean micrograms. The term “h”, as used herein, is intended to mean hour. The term “min”, as used herein, is intended to mean minute. The term “M”, as used herein, is intended to mean molar. The term “μL”, as used herein, is intended to mean microliter. The term “μM”, as used herein, is intended to mean micromolar. The term “nM”, as used herein, is intended to mean nanomolar. The term “N”, as used herein, is intended to mean normal. The term “amu”, as used herein, is intended to mean atomic mass unit. The term “° C.”, as used herein, is intended to mean degree Celsius. The term “wt/wt”, as used herein, is intended to mean weight/weight. The term “v/v”, as used herein, is intended to mean volume/volume. The term “MS”, as used herein, is intended to mean mass spectroscopy. The term “HPLC”, as used herein, is intended to mean high performance liquid chromatograph. The term “RT”, as used herein, is intended to mean room temperature. The term “e.g.”, as used herein, is intended to mean example. The term “N/A”, as used herein, is intended to mean not tested.

As used herein, the expression “pharmaceutically acceptable salt” refers to pharmaceutically acceptable organic or inorganic salts of a compound of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, or pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counterions. Hence, a pharmaceutically acceptable salt can have one or more charged atoms and/or one or more counterion. As used herein, the expression “pharmaceutically acceptable solvate” refers to an association of one or more solvent molecules and a compound of the invention. Examples of solvents that form pharmaceutically acceptable solvates include, but are not limited to, water, isopropanol, ethanol, methanol, DMSO, ethyl acetate, acetic acid, and ethanolamine. As used herein, the expression “pharmaceutically acceptable hydrate” refers to a compound of the invention, or a salt thereof, that further includes a stoichiometric or non-stoichiometric amount of water bound by non-covalent intermolecular forces.

Cytokines, Inflammatory Markers, and Lps-Mediated Responses Associated Diseases, Disorders, or Conditions

An immunomodulatory agent as described herein can treat, reduce, or prevent a disease, disorder, or condition associated with inflammation or an immune response. For example, diseases associated with inflammation or an immune response can include ischaemia-reperfusion, cardiovascular infarction, inflammatory bowel disease, psoriasis, multiple sclerosis, rheumatoid arthritis autoinflammatory disease, or an autoimmune disease. As another example, the disease, disorder, or condition can be ischaemia-reperfusion in the heart, kidney, or brain or a tissue injury caused by ischaemia-reperfusion in the heart, kidney, or brain, or myocardial injury, where the tissue injury can occur during reperfusion.

The immunomodulatory agents as described herein can treat a disease, disorder, or condition associated with inflammation or an immune response by modulating cytokines and inflammatory markers. For example, the immunomodulatory agents have been shown to modulate the expression or secretion of Casp1, iNOS, HIF-1α, pro-IL-1β, ASC, NLRP3, NOS2, iNOS, IL6, IL12B, IFNB1, IL-12p70, IL-6, IL-1β, IL-12β, NO, GM-CSF, IL-17, or IL-18. As another example, the immunomodulatory agents as described herein can treat a disease, disorder, or condition associated with increased expression or secretion of Casp1, iNOS, HIF-1α, pro-IL-1β, ASC, NLRP3, NOS2, iNOS, IL6, IL12B, IFNB1, IL-12p70, IL-6, IL-1β, IL-12β, NO, GM-CSF, IL-17, or IL-18.

Diseases, disorders, and conditions that can be treated by the immunomodulatory agents include: adult and juvenile Still disease; asthma; allergy; Alzheimer's disease; age-related macular degeneration; antisynthetase syndrome; autoinflammatory disease; autoimmune disease; autoimmune response; Behcet disease; Blau syndrome; cancer; cardiovascular infarction; chronic infantile neurological cutaneous and articular (CINCA) syndrome; chronic recurrent multifocal osteomyelitis; cinca syndrome; classic autoinflammatory diseases; cryopyrin-associated autoinflammatory syndromes (CAPS); deficiency in IL-1 receptor antagonist (DIRA); diabetes mellitus; Erdheim-Chester syndrome (histiocytosis); extrapulmonary tuberculosis; familial atypical mycobacteriosis; familial cold autoinflammatory syndrome (FCAS); gastric cancer Risk after H. pylori Infection; Guillain-Barré syndrome; Hashimoto's thyroiditis; heart failure; hepatic fibrosis; Huntington's disease; hyper IgD syndrome (HIDS); hypoxia; ischaemia-reperfusion; immunodeficiency 29; inflammation; inflammation by HIV; inflammatory bowel disease (IBD); macrophage activation syndrome (MAS); mycobacteriosis; Miller-Fisher syndrome; Muckle-Wells syndrome (MWS); multiple sclerosis (MS); neonatal-onset multisystem inflammatory disease (NOMID); neuropathic pain; N syndrome; osteoarthritis; osteoporosis; Periodontal Disease; periodic fever, aphthous stomatitis, pharyngitis, adenitis syndrome (PFAPA); postmyocardial infarction heart failure; psoriasis; recurrent idiopathic pericarditis; recurrent pericarditis; relapsing chondritis; relapsing-remitting multiple sclerosis; rheumatoid arthritis (RA); Sapho Syndrome; Schnitzler syndrome; secondary progressive multiple sclerosis; septic shock; smoldering myeloma; Sweet syndrome; synovitis, acne, pustulosis, hyperostosis, osteitis (SAPHO); systemic juvenile rheumatoid arthritis; familial Mediterranean fever (FMF); pyogenic arthritis; pyoderma gangrenosum, acne (PAPA); TNF receptor-associated periodic syndrome (TRAPS); type 2 diabetes; urate crystal arthritis (gout); or urticarial vasculitis.

Cytokines are considered to be in a broad and loose category of small proteins (˜5-20 kDa) that are important in cell signaling. Their release has an effect on the behavior of cells around them. It can be said that cytokines are involved in autocrine signaling, paracrine signaling and endocrine signaling as immunomodulating agents. Their definite distinction from hormones is still part of ongoing research. Cytokines are generally known to include chemokines, interferons, interleukins, lymphokines, and tumour necrosis factors but generally not hormones or growth factors (despite some overlap in the terminology). Cytokines can be produced by a broad range of cells, including immune cells like macrophages, B lymphocytes, T lymphocytes and mast cells, as well as endothelial cells, fibroblasts, and various stromal cells; a given cytokine may be produced by more than one type of cell.

Cytokines can act through receptors, and are especially important in the immune system. Cytokines can modulate the balance between humoral and cell-based immune responses, and they can regulate the maturation, growth, or responsiveness of particular cell populations. Some cytokines can enhance or inhibit the action of other cytokines in complex ways.

Cytokines are different from hormones, which can also be important in cell signaling molecules, in that hormones circulate in less variable concentrations and hormones tend to be made by specific kinds of cells.

Cytokines can be important in health and disease, specifically in host responses to infection, immune responses, inflammation, trauma, sepsis, cancer, or reproduction.

The immunomodulatory agents as described herein can treat a disease, disorder, or condition associated with increased expression or secretion of Casp1, iNOS, HIF-1α, pro-IL-1β, ASC, NLRP3, NOS2, iNOS, IL6, IL12B, IFNB1, IL-12p70, IL-6, IL-1β, IL-12β, NO, GM-CSF, IL-17, or IL-18.

Interleukin 1 beta (IL-1β)/1L1B associated diseases.

Interleukin 1 beta (IL1β), including pro-IL-1β, also known as leukocytic pyrogen, leukocytic endogenous mediator, mononuclear cell factor, lymphocyte activating factor or other names, is a cytokine protein that in humans is encoded by the IL1B gene. There are two genes for interleukin-1 (IL-1): IL-1 alpha and IL-1 beta. IL-1β precursor is cleaved by cytosolic caspase 1 (interleukin 1 beta convertase) to form mature IL-1β.

Increased production of IL-1β can causes a number of different autoinflammatory syndromes, most notably the monogenic conditions referred to as Cryopyrin-Associated Autoinflammatory Syndromes (CAPS), due to mutations in the inflammasome receptor NLRP3 which triggers processing of IL-1β.

IL-1β can be associated with a number of autoinflammatory diseases. For these, neutralization of IL-1β results in a rapid and sustained reduction in disease severity. Treatment for autoimmune diseases often includes immunosuppressive drugs whereas neutralization of IL-1β is mostly anti-inflammatory.

For example IL-1β implicated diseases can include gout, type 2 diabetes, heart failure, recurrent pericarditis, rheumatoid arthritis, and smoldering myeloma also are responsive to IL-1β neutralization.

It is well established that IL-1β is implicated in numerous inflammatory diseases (see e.g., Dinarello, Blood. 2011 April 7; 117(14): 3720-3732). For example, the following conditions can be treated with blocking or reduction in IL-1β: Classic autoinflammatory diseases; Familial Mediterranean fever (FMF); Pyogenic arthritis, pyoderma gangrenosum, acne (PAPA); Cryopyrin-associated periodic syndromes (CAPS); Hyper IgD syndrome (HIDS); Adult and juvenile Still disease; Schnitzler syndrome; TNF receptor-associated periodic syndrome (TRAPS) Blau syndrome; Sweet syndrome; Deficiency in IL-1 receptor antagonist (DIRA) Recurrent idiopathic pericarditis; Macrophage activation syndrome (MAS) Urticarial vasculitis; Antisynthetase syndrome; Relapsing chondritis; Behcet disease; Erdheim-Chester syndrome (histiocytosis) Synovitis, acne, pustulosis, hyperostosis, osteitis (SAPHO); Rheumatoid arthritis; Periodic fever, aphthous stomatitis, pharyngitis, adenitis syndrome (PFAPA) Urate crystal arthritis (gout); Type 2 diabetes; Smoldering multiple myeloma; Postmyocardial infarction heart failure; or Osteoarthritis.

Diseases associated with IL-1β include Gastric Cancer Risk After H. Pylori Infection and Periodontal Disease.

Interleukin 17 (IL-17) associated diseases.

Interleukin 17 is a pro-inflammatory cytokine produced by T-helper cells, gamma-delta T cells and subsets of innate lymphoid cells (Sutton et al, EJI 2012; Klose and Artis, Nat Immunol, 2016), and is induced and/or promoted by cytokines including IL-6, IL-23, IL-1β, or TGFβ. To elicit its functions, IL-17 binds to a type I cell surface receptor called IL-17R of which there are at least three variants IL17RA, IL17RB, and IL17RC. IL-17 acts as a potent mediator in delayed-type reactions by increasing chemokine production in various tissues. Signaling from IL-17 recruits monocytes and neutrophils to the site of inflammation in response to invasion by pathogens, similar to Interferon gamma. In promoting inflammation, IL-17 has been demonstrated to act synergistically with tumor necrosis factor and interleukin-1. This activity can also be redirected towards the host and result in various autoimmune disorders that involve chronic inflammation, such as the skin disorder psoriasis.

IL-17 is implicated in numerous inflammatory diseases (see e.g., psoriasis, vitiligo, allergies, autoimmune disease, rheumatoid arthritis, inflammatory bowel disease, multiple sclerosis, or asthma) (Wang et al PlosOne 2011).

Interleukin 18 (IL-18)/IL18 associated diseases.

IL-18 has been shown to induce severe inflammatory reactions, which suggests its role in certain inflammatory disorders. For example, IL-18 has been implicated in age-related macular degeneration, Hashimoto's thyroiditis, Alzheimer's disease.

Diseases associated with IL18 include Adult-Onset Still's Disease and Sapho Syndrome.

Casp1 associated diseases.

Caspase-1/Interleukin-1 converting enzyme (ICE) plays a central role in cell immunity as an inflammatory response initiator. Caspase-1 has also been shown to induce necrosis and may also function in various developmental stages. Studies suggest a role in the pathogenesis of Huntington's disease. Alternative splicing of the gene results in five transcript variants encoding distinct isoforms. Recent studies implicated caspase-1 in promoting CD4 T-cell death and inflammation by HIV, two signature events that fuel HIV disease progression to AIDS.

Inducible Nitric oxide synthases (iNOS), NOS2, and NO associated diseases.

Nitric oxide synthases (NOSs) are a family of enzymes catalyzing the production of nitric oxide (NO) from L-arginine. NO is an important cellular signaling molecule. It helps modulate vascular tone, insulin secretion, airway tone, and peristalsis, and is involved in angiogenesis and neural development. It may function as a retrograde neurotransmitter. Nitric oxide is mediated in mammals by the calcium-calmodulin controlled isoenzymes eNOS (endothelial NOS) and nNOS (neuronal NOS). The inducible isoform, iNOS, is involved in immune response, binds calmodulin at physiologically relevant concentrations, and produces NO as an immune defense mechanism, as NO is a free radical with an unpaired electron. It is the proximate cause of septic shock and may function in autoimmune disease.

HIF-1α associated diseases.

Hypoxia-inducible factor 1-alpha, also known as HIF-1-alpha, is a subunit of a heterodimeric transcription factor hypoxia-inducible factor 1 (HIF-1) that is encoded by the HIF1A gene. It is a basic helix-loop-helix PAS domain containing protein, and is considered as the master transcriptional regulator of cellular and developmental response to hypoxia. The dysregulation and overexpression of HIF1A by either hypoxia or genetic alternations have been heavily implicated in cancer biology, as well as a number of other pathophysiologies, specifically in areas of vascularization and angiogenesis, energy metabolism, cell survival, and tumor invasion. Two other alternative transcripts encoding different isoforms have been identified.

Pycard (ASC) associated diseases.

Apoptosis-associated speck-like protein containing a CARD or ASC is a protein that in humans is encoded by the PYCARD gene.

This gene encodes an adaptor protein that is composed of two protein-protein interaction domains: an N-terminal PYRIN-PAAD-DAPIN domain (PYD) and a C-terminal caspase-recruitment domain (CARD). The PYD and CARD domains are members of the six-helix bundle death domain-fold superfamily that mediates assembly of large signaling complexes in the inflammatory and apoptotic signaling pathways via the activation of caspase. In normal cells, this protein is localized to the cytoplasm; however, in cells undergoing apoptosis, it forms ball-like aggregates near the nuclear periphery. Two transcript variants encoding different isoforms have been found for this gene.

Diseases associated with PYCARD include Chronic Recurrent Multifocal Osteomyelitis and Cinca Syndrome.

NLRP3 associated diseases.

NACHT, LRR and PYD domains-containing protein 3 (NALP3) also known by cryopyrin is a protein that in humans is encoded by the NLRP3 gene located on the long arm of chromosome 1.

NALP3 is expressed predominantly in macrophages and as a component of the inflammasome, detects products of damaged cells such as extracellular ATP and crystalline uric acid. Activated NALP3 in turn triggers an immune response. Mutations in the NLRP3 gene are associated with a number of organ specific autoimmune diseases.

Mutations in the NLRP3 gene have been associated with a spectrum of dominantly inherited autoinflammatory diseases called cryopyrin-associated periodic syndrome (CAPS). This includes familial cold autoinflammatory syndrome (FCAS), Muckle-Wells syndrome (MWS), chronic infantile neurological cutaneous and articular (CINCA) syndrome, and neonatal-onset multisystem inflammatory disease (NOMID).

Defects in this gene have also been linked to familial Mediterranean fever. In addition, the NALP3 inflammasome has a role in the pathogenesis of gout and neuroinflammation occurring in protein-misfolding diseases, such as Alzheimer's, Parkinson's, and Prion diseases.

Deregulation of NALP3 has been connected with carcinogenesis. For example, all the components of the NALP3 inflammasome are downregulated or completely lost in human hepatocellular carcinoma.

Diseases associated with NALP3 are familial cold autoinflammatory syndrome (FCAS), Muckle-Wells syndrome (MWS), chronic infantile neurological cutaneous and articular (CINCA) syndrome, and neonatal-onset multisystem inflammatory disease (NOMID).

IL-6 associated diseases.

Interleukin 6 (IL-6) is an interleukin that acts as both a pro-inflammatory cytokine and an anti-inflammatory myokine. In humans, it is encoded by the IL6 gene.

Interleukin 6 is secreted by B cells, T cells, and macrophages to stimulate immune response, e.g. during infection and after trauma, especially burns or other tissue damage leading to inflammation. IL-6 also plays a role in fighting infection, as IL-6 has been shown in mice to be required for resistance against bacterium Streptococcus pneumoniae.

In addition, osteoblasts secrete IL-6 to stimulate osteoclast formation. Smooth muscle cells in the tunica media of many blood vessels also produce IL-6 as a pro-inflammatory cytokine. IL-6's role as an anti-inflammatory cytokine is mediated through its inhibitory effects on TNF-alpha and IL-1, and activation of IL-1ra and IL-10.

IL-6 is associated with and stimulates the inflammatory and auto-immune processes in many diseases such as diabetes, atherosclerosis, depression, Alzheimer's Disease, systemic lupus erythematosus, multiple myeloma, prostate cancer, Behçet's disease, inflammatory bowel disease (Neurath, Nat Rev Immunol, 2014), rheumatoid arthritis, vitiligo, and systemic sclerosis (O'Reilly et al, Clin and Translational Immunol, 2013).

IL-6 has been associated with diabetes mellitus and systemic juvenile rheumatoid arthritis.

IFNB associated diseases.

Interferons (IFNs) are a group of signaling proteins made and released by host cells in response to the presence of several pathogens, such as viruses, bacteria, parasites, and also tumor cells. In a typical scenario, a virus-infected cell will release interferons causing nearby cells to heighten their anti-viral defenses.

IFNs belong to the large class of proteins known as cytokines, molecules used for communication between cells to trigger the protective defenses of the immune system that help eradicate pathogens. Interferons are named for their ability to “interfere” with viral replication by protecting cells from virus infections. IFNs also have various other functions: they activate immune cells, such as natural killer cells and macrophages; they increase host defenses by up-regulating antigen presentation by virtue of increasing the expression of major histocompatibility complex (MHC) antigens. Certain symptoms of infections, such as fever, muscle pain and “flu-like symptoms”, are also caused by the production of IFNs and other cytokines.

Overactivation of type I interferon secretion is linked to autoimmune diseases. Interferon beta is a protein that in humans is encoded by the IFNB1 gene. Diseases associated with IFNB1 include Relapsing-Remitting Multiple Sclerosis and Secondary Progressive Multiple Sclerosis.

IL-12p70 and IL-12β/IL12B associated diseases.

Subunit beta of interleukin 12 (also known as IL-12B, natural killer cell stimulatory factor 2, cytotoxic lymphocyte maturation factor p40, or interleukin-12 subunit p40) is a protein that in humans is encoded by the IL12B gene. IL-12B is a common subunit of interleukin 12 and Interleukin 23.

This gene encodes a subunit of interleukin 12, a cytokine that acts on T and natural killer cells, and has a broad array of biological activities. Interleukin 12 is a disulfide-linked heterodimer composed of the 40 kD cytokine receptor like subunit encoded by this gene, and a 35 kD subunit encoded by IL12A. This cytokine is expressed by activated macrophages that serve as an essential inducer of Th1 cells development. This cytokine has been found to be important for sustaining a sufficient number of memory/effector Th1 cells to mediate long-term protection to an intracellular pathogen. Overexpression of this gene was observed in the central nervous system of patients with multiple sclerosis (MS), suggesting a role of this cytokine in the pathogenesis of the disease. The promoter gene polymorphism of this gene has been reported to be associated with the severity of atopic and non-atopic asthma in children.

Interleukin 12 (IL-12) is an interleukin that is naturally produced by dendritic cells, macrophages, neutrophils, and human B-lymphoblastoid cells (NC-37) in response to antigenic stimulation. IL-12 is composed of a bundle of four alpha helices. It is a heterodimeric cytokine encoded by two separate genes, IL-12A (p35) and IL-12B (p40). The active heterodimer (referred to as ‘p70’), and a homodimer of p40 are formed following protein synthesis.

IL-12 is linked with autoimmunity. Administration of IL-12 to people suffering from autoimmune diseases was shown to worsen the autoimmune phenomena. This is believed to be due to its key role in induction of Th1 immune responses. In contrast, IL-12 gene knock-out in mice or a treatment of mice with IL-12 specific antibodies ameliorated the disease.

Interleukin 12 (IL-12) is produced by activated antigen-presenting cells (dendritic cells, macrophages). It promotes the development of Th1 responses and is a powerful inducer of IFNγ production by T and NK cells.

Other diseases associated with IL12B include Immunodeficiency 29, Mycobacteriosis and Familial Atypical Mycobacteriosis.

IL-12p70 has been ishown to be overexpressed in Crohn's disease.

Dysregulated expression of IL-12 p40 can lead to prolonged, unresolved inflammation manifesting into chronic inflammatory disorders such as inflammatory bowel disease (IBD).

Overexpression IL12B was observed in the central nervous system of patients with multiple sclerosis (MS).

Diseases associated with IL12RB1 include Immunodeficiency 30 and Familial Atypical Mycobacteriosis.

GM-CSF associated diseases.

Granulocyte-macrophage colony-stimulating factor (GM-CSF), also known as colony stimulating factor 2 (CSF2), is a monomeric glycoprotein secreted by macrophages, T cells, B cells, mast cells, NK cells, endothelial cells and fibroblasts that functions as a cytokine. The pharmaceutical analogs of naturally occurring GM-CSF are called sargramostim and molgramostim.

GM-CSF is found in high levels in joints with rheumatoid arthritis, in the cerebrospinal fluid of MS patients and in the serum of patients with acute aortic aneurysm. Also, its receptor is highly expressed in subsets of myeloid cells in patients with rheumatoid arthritis and psoriatic arthritis. GM-CSF can activate microglial cells that promote inflammation of the central nervous system. Targeting GM-CSF may reduce inflammation or damage and could be beneficial for patients with rheumatoid arthritis, MS, plaque psoriasis, and asthma (Wicks and Roberts, Nat Rev Rheumatology, 2016).

P2rx7.

P2X purinoceptor 7 is a protein that in humans is encoded by the P2RX7 gene.

The product of this gene belongs to the family of purinoceptors for ATP. Multiple alternatively spliced variants which would encode different isoforms have been identified although some fit nonsense-mediated decay criteria.

The receptor is found in the central and peripheral nervous systems, in microglia, in macrophages, in uterine endometrium, and in the retina. The P2X7 receptor also serves as a pattern recognition receptor for extracellular ATP-mediated apoptotic cell death, regulation of receptor trafficking, mast cell degranulation, and inflammation.

Diseases associated with P2RX7 include Extrapulmonary Tuberculosis and N Syndrome. Among its related pathways are Peptide ligand-binding receptors and Nucleotide-binding domain, leucine rich repeat containing receptor (NLR) signaling pathways.

Microglial P2X7 receptors are thought to be involved in neuropathic pain because blockade or deletion of P2X7 receptors results in decreased responses to pain, as demonstrated in vivo.

Moreover, P2X7 receptor signaling increases the release of proinflammatory molecules such as IL-1β, IL-6, and TNF-α. In addition, P2X7 receptors have been linked to increases in proinflammatory cytokines such as CXCL2 and CCL3. Interestingly, P2X7 receptors are also linked to P2X4 receptors, which are also associated with neuropathic pain mediated by microglia.

P2RX7 has also been linked to osteoporosis. Mutations in this gene have been associated to low lumbar spine bone mineral density and accelerated bone loss in post-menopausal women.

P2RX7 has also been linked to diabetes. The ATP/P2X7R pathway may trigger T-cell attacks on the pancreas, rendering it unable to produce insulin. This autoimmune response may be an early mechanism by which the onset of diabetes is caused.

P2RX7 has also been linked to hepatic fibrosis. One study in mice showed that blockade of P2X7 receptors attenuates onset of liver fibrosis.

LPS-mediated immune response associated diseases.

The immunomodulatory agents as described herein have been shown to suppress a lipopolysaccharide (LPS)-mediated immune response. Diseases associated with LPS-mediated immune response that can be treated with immunomodulatory agents can include: autoimmune disease and responses, MS flare ups, Guillain-Barré syndrome and a variant of Guillain-Barré called Miller-Fisher syndrome.

CD4 T Cells

The T helper cells (Th cells) are a type of T cell that can play an important role in the immune system, particularly in the adaptive immune system. They help the activity of other immune cells by releasing T cell cytokines. These cells can help suppress or regulate immune responses. They are essential in B cell antibody class switching, in the activation and growth of cytotoxic T cells, and in maximizing bactericidal activity of phagocytes such as macrophages.

Mature Th cells express the surface protein CD4 and are referred to as CD4+ T cells. Such CD4+ T cells are generally treated as having a pre-defined role as helper T cells within the immune system. For example, when an antigen-presenting cell expresses an antigen on MHC class II, a CD4+ cell will aid those cells through a combination of cell to cell interactions (e.g. CD40 (protein) and CD40L) and through cytokines.

TH17 Cells

T helper 17 cells (Th17) are a subset of pro-inflammatory T helper cells that can be defined by their production of interleukin 17 (IL-17). They are related to T regulatory cells and the signals that cause Th17s to differentiate are currently thought to inhibit Treg differentiation. Th17s can be developmentally distinct from Th1 and Th2 lineages. Th17 cells can play an important role in maintaining mucosal barriers and contributing to pathogen clearance at mucosal surfaces, but they have also been implicated in autoimmune and inflammatory disorders. The loss of Th17 cell populations at mucosal surfaces has been linked to chronic inflammation and microbial translocation.

Molecular Engineering

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host cell or, if from the same source, is modified from its original form. Thus, a heterologous gene in a host cell includes a gene that is endogenous to the particular host cell but has been modified through, for example, the use of DNA shuffling. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a promoter operably linked to a transcribable nucleic acid molecule operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

Design, generation, and testing of the variant nucleotides, and their encoded polypeptides, having the above required percent identities and retaining a required activity of the expressed protein is within the skill of the art. For example, directed evolution and rapid isolation of mutants can be according to methods described in references including, but not limited to, Link et al. (2007) Nature Reviews 5(9), 680-688; Sanger et al. (1991) Gene 97(1), 119-123; Ghadessy et al. (2001) Proc Natl Acad Sci USA 98(8) 4552-4557. Thus, one skilled in the art could generate a large number of nucleotide and/or polypeptide variants having, for example, at least 95-99% identity to the reference sequence described herein and screen such for desired phenotypes according to methods routine in the art.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2 or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine), Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tₘ) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA:DNA sequence can be determined using the following formula: Tₘ=81.5° C.+16.6(log₁₀[Na⁺])+0.41(fraction G/C content)−0.63(% formamide)−(600/l). Furthermore, the Tₘ of a DNA:DNA hybrid is decreased by 1-1.5° C. for every 1% decrease in nucleotide identity (see e.g., Sambrook and Russel, 2006).

Host cells can be transformed using a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Methods of down-regulation or silencing genes are known in the art. For example, expressed protein activity can be down-regulated or eliminated using antisense oligonucleotides, protein aptamers, nucleotide aptamers, and RNA interference (RNAi) (e.g., small interfering RNAs (siRNA), short hairpin RNA (shRNA), and micro RNAs (miRNA) (see e.g., Fanning and Symonds (2006) Handb Exp Pharmacol. 173, 289-303G, describing hammerhead ribozymes and small hairpin RNA; Helene, C., et al. (1992) Ann. N.Y. Acad. Sci. 660, 27-36; Maher (1992) Bioassays 14(12): 807-15, describing targeting deoxyribonucleotide sequences; Lee et al. (2006) Curr Opin Chem Biol. 10, 1-8, describing aptamers; Reynolds et al. (2004) Nature Biotechnology 22(3), 326-330, describing RNAi; Pushparaj and Melendez (2006) Clinical and Experimental Pharmacology and Physiology 33(5-6), 504-510, describing RNAi; Dillon et al. (2005) Annual Review of Physiology 67, 147-173, describing RNAi; Dykxhoorn and Lieberman (2005) Annual Review of Medicine 56, 401-423, describing RNAi). RNAi molecules are commercially available from a variety of sources (e.g., Ambion, TX; Sigma Aldrich, MO; Invitrogen). Several siRNA molecule design programs using a variety of algorithms are known to the art (see e.g., Cenix algorithm, Ambion; BLOCK-iT™ RNAi Designer, Invitrogen; siRNA Whitehead Institute Design Tools, Bioinofrmatics & Research Computing). Traits influential in defining optimal siRNA sequences include G/C content at the termini of the siRNAs, Tm of specific internal domains of the siRNA, siRNA length, position of the target sequence within the CDS (coding region), and nucleotide content of the 3′ overhangs.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A.R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intramuscular, intraperitoneal, intravenous, subcutaneous, intranasal, epidural, ophthalmic, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for treatment of the disease, disorder, or condition.

Therapeutic Methods

Also provided is a process of treating a disease, disorder, or condition associated with inflammation or an immune response in a subject in need and administration of a therapeutically effective amount of an immunomodulatory agent comprising itaconate or malonate, or a derivative thereof, so as to reduce immune response or result in anti-inflammatory effects.

Methods described herein are generally performed on a subject in need thereof. A subject in need of the therapeutic methods described herein can be a subject having, diagnosed with, suspected of having, or at risk for developing a disease, disorder, or condition associated with inflammation or an immune response, such as ischemia-reperfusion injury, cardiovascular infarction, inflammatory bowel disease, psoriasis, multiple sclerosis, rheumatoid arthritis, autoinflammatory disease, or an autoimmune disease. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of an immunomodulatory agent comprising itaconate, malonate, or a derivative thereof is, for example, that amount that would cause the desired therapeutic effect in a subject while minimizing undesired side effects. In various embodiments, an effective amount of an immunomodulatory agent comprising itaconate, malonate, or a derivative thereof described herein can substantially inhibit inflammation or an immune response, slow the progress of a disease, disorder, or condition associated with inflammation or an immune response, or limit the development of a disease, disorder, or condition associated with inflammation or an immune response.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of itaconate, malonate, or a derivative thereof can be employed in pure form or, where such forms exist, in pharmaceutically acceptable salt form and with or without a pharmaceutically acceptable excipient. For example, the compounds of the present disclosure can be administered, at a reasonable benefit/risk ratio applicable to any medical treatment, in a sufficient amount to reduce or prevent inflammation or an immune response.

The amount of an immunomodulatory agent described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of itaconate, malonate, or a derivative thereof can occur as a single event or over a time course of treatment. For example, itaconate, malonate, or a derivative thereof can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a disease, disorder, or condition associated with inflammation or an immune response.

An immunomodulating agent, as described herein, can be administered simultaneously or sequentially with another agent, such as an antibiotic, an antiinflammatory, or another agent. For example, an immunomodulating agent can be administered simultaneously with another agent, such as an antibiotic or an antiinflammatory. Simultaneous administration can occur through administration of separate compositions, each containing one or more of an immunomodulating agent, an antibiotic, an antiinflammatory, or another agent. Simultaneous administration can occur through administration of one composition containing two or more of an immunomodulating agent, an antibiotic, an antiinflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to a composition comprising an immunomodulating agent. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Itaconate is an Endogenous Metabolic Regulator of the Inflammatory Functions of Succinate

In the context of inflammation, macrophage activation is accompanied by marked changes in metabolism, including upregulation of glycolytic flux, disruption of the TCA cycle at the isocitrate to 2-oxoglutarate transition (Idh1), and accumulation of succinate. While a significant portion of the metabolic adaptation is transcriptionally controlled (e.g., via modulation of Nos2, Cox2, and Idh1 transcript levels), succinate provides an important example of a metabolite acting in a regulatory role, affecting major inflammatory pathways in both immune and non-immune cell types, by controlling IL-1β production, HIF-1α activity and ROS levels. However, the mechanism(s) regulating succinate levels during macrophage activation have not been identified to date.

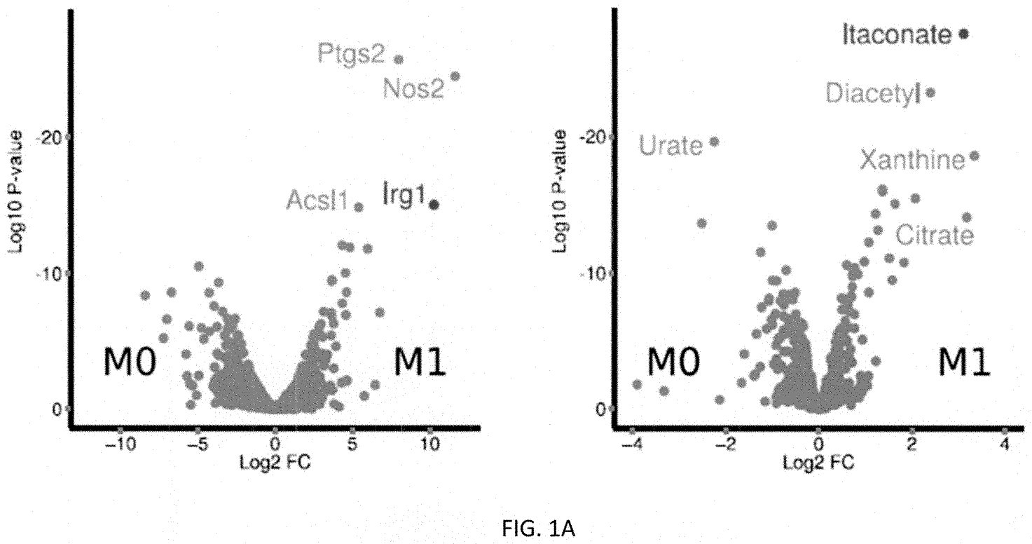

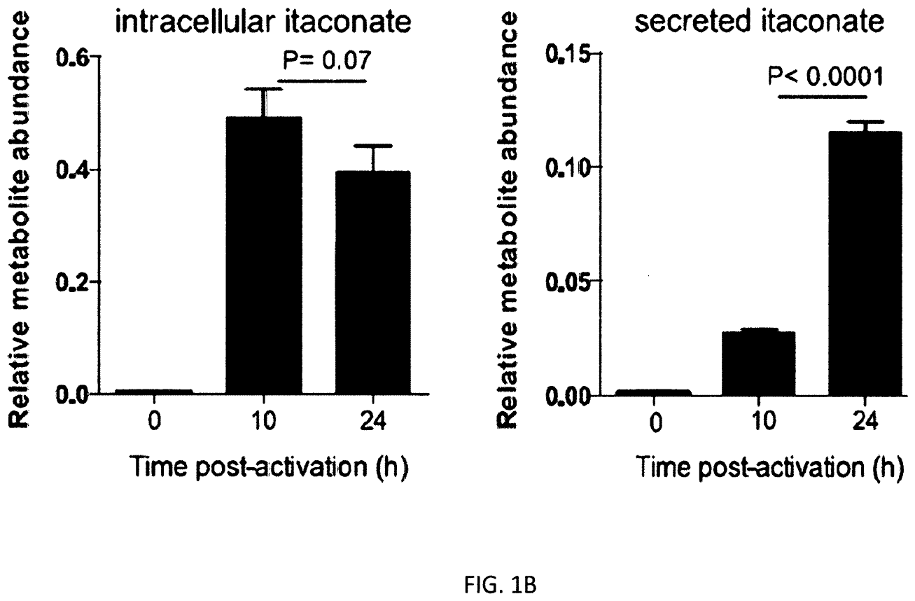

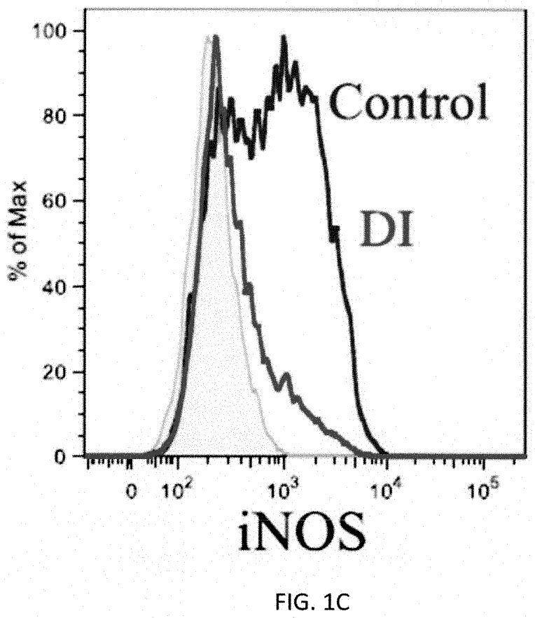

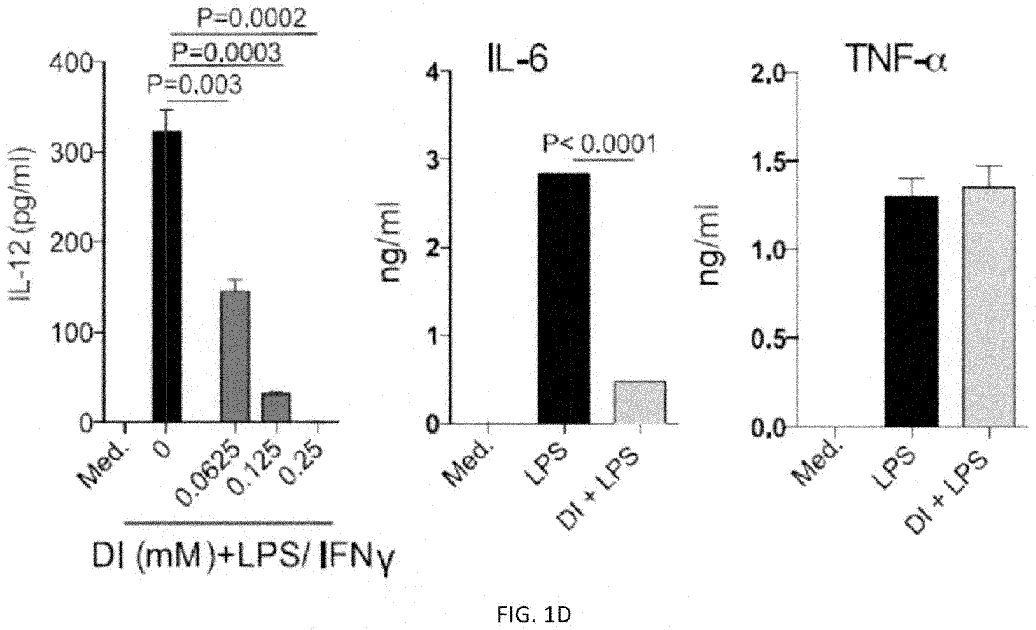

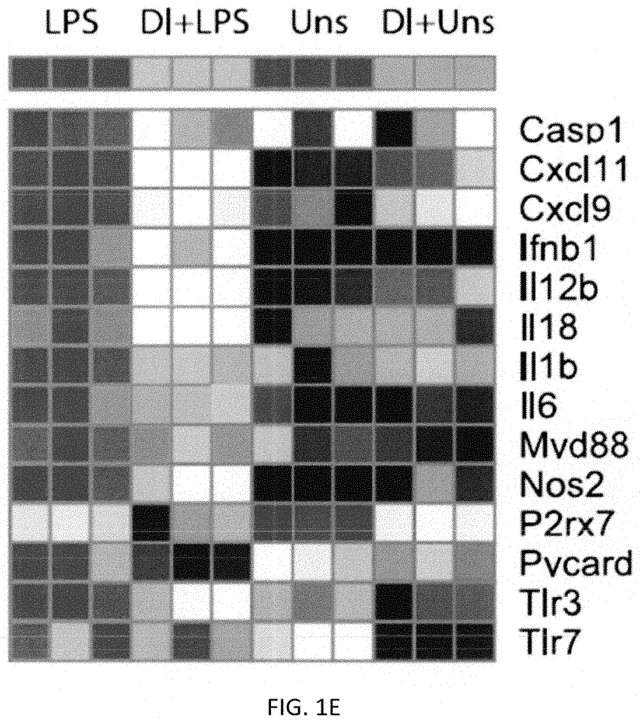

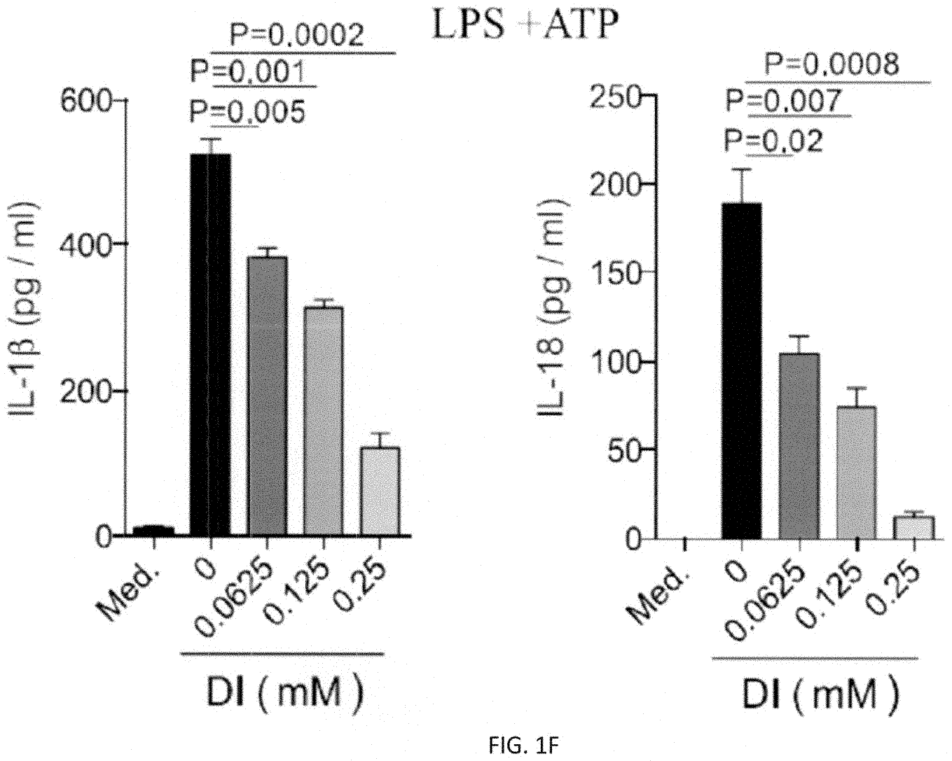

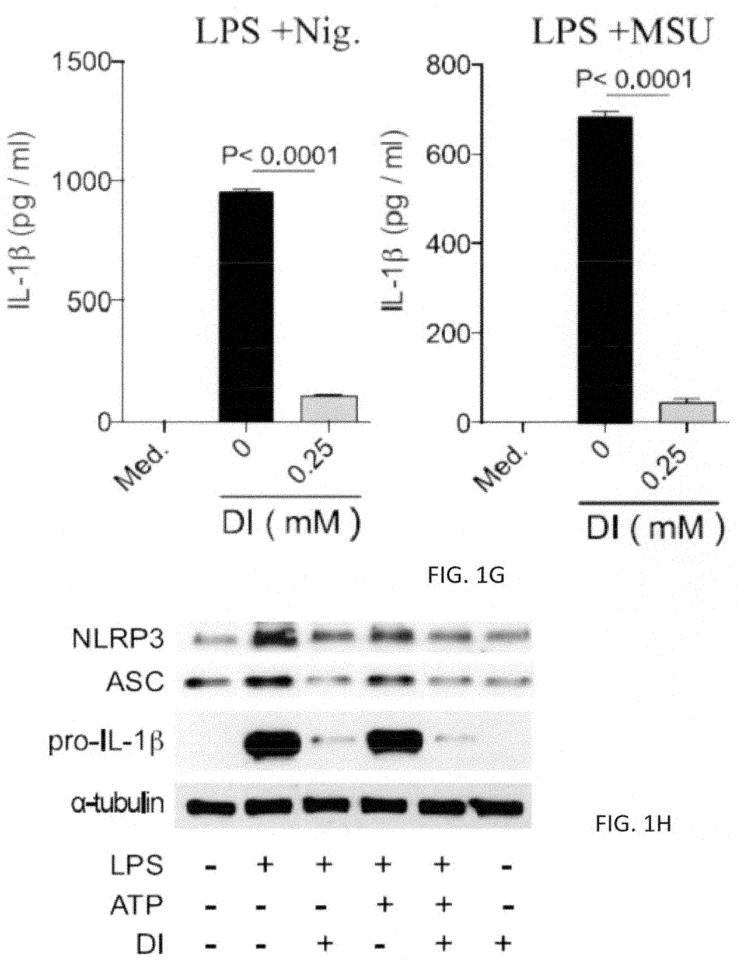

During macrophage activation, the TCA cycle break point at Idh1 redirects the metabolic flux towards production of cytosolic acetyl-CoA and itaconate. Indeed, after lipopolysaccharide (LPS+IFN-γ) stimulation, Irg1, the mitochondria-associated enzyme regulating itaconate production, is highly induced, and itaconate accumulates to high levels within cells (see e.g., FIG. 1A). Metabolomic profiling of culture supernatants from activated bone marrow derived macrophages (BMDMs) revealed and confirmed that itaconate also was secreted (see e.g., FIG. 1B), with 70-fold higher levels measured within 24 h of stimulation with LPS and IFN-γ. As circulating metabolites can influence innate and adaptive immune responses, the magnitude of itaconate production and secretion prompted the investigation of its potential regulatory role during macrophage activation.

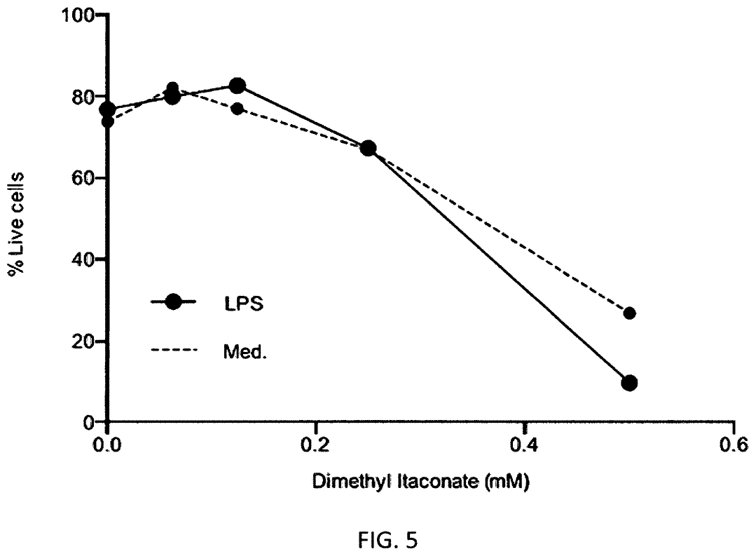

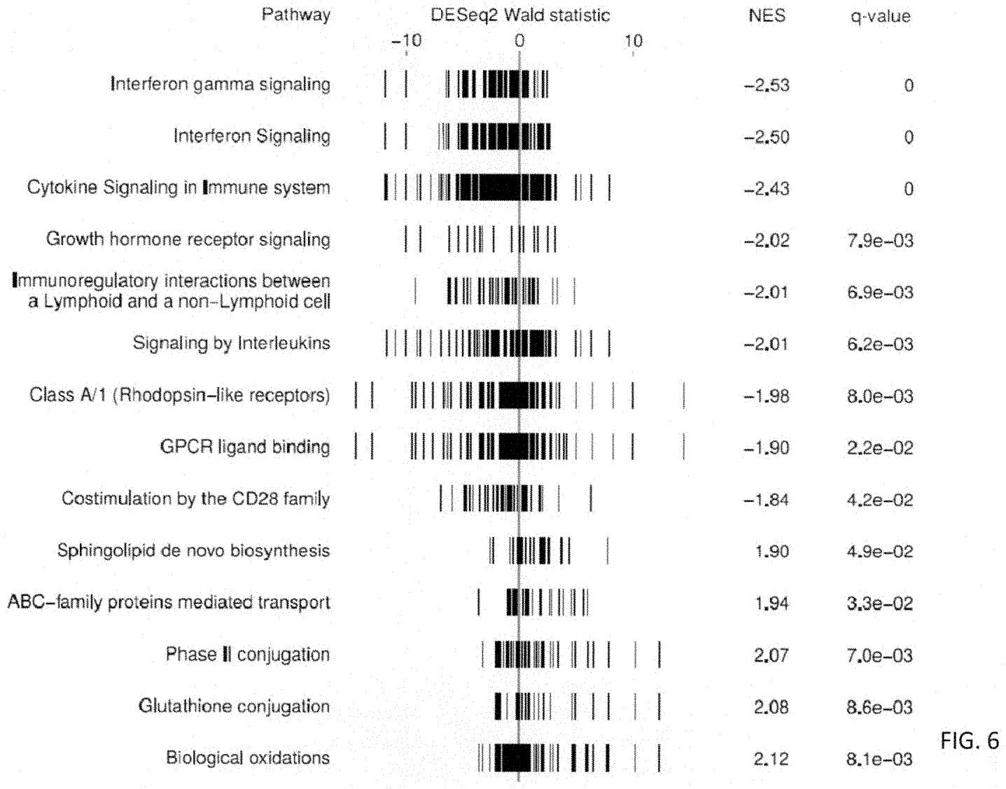

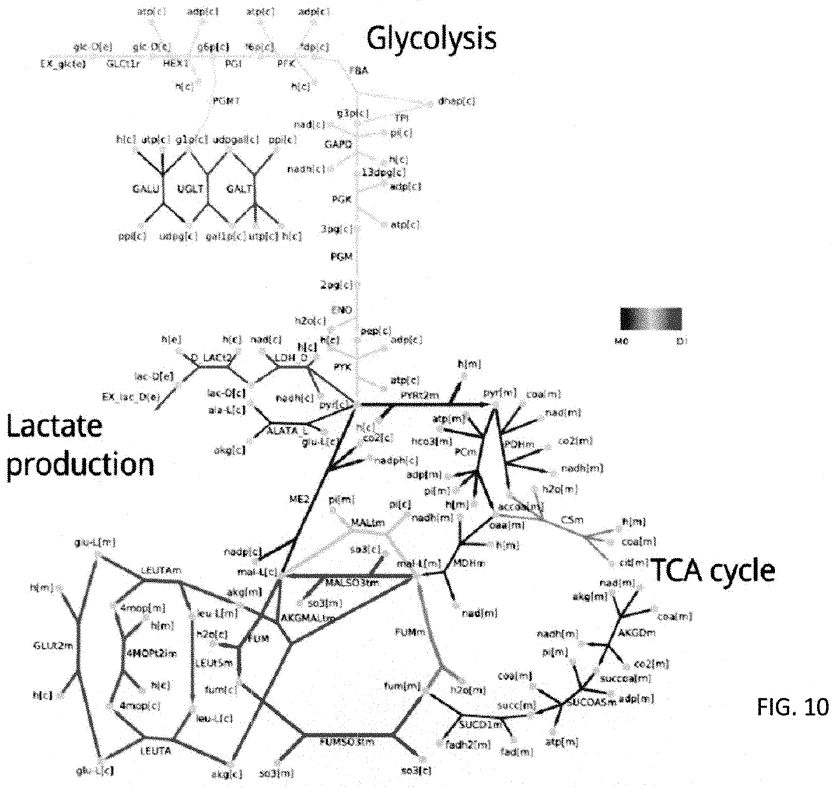

The effect of exogenously added itaconate on the inflammatory response induced after LPS or LPS+IFN-γ stimulation was tested. Mouse bone marrow-derived macrophage (BMDM) was treated with physiologically relevant doses of dimethyl itaconate (DI) (see e.g., FIG. 5), a membrane-permeable nonionic form of itaconate. Pretreatment with DI suppressed iNOS protein expression (see e.g., FIG. 10) and IL-12p70 and IL-6 secretion (see e.g., FIG. 1D) thereby interfering with activation of pro-inflammatory macrophages. In contrast, TNF-α production remained unchanged (see e.g., FIG. 1D), indicating that the effects of DI-treatment were not due to global inhibition of NF-KB-dependent gene expression. To determine the specific pathways affected by itaconate, a global transcriptional profiling by RNA-seq on BMDM pre-treated with DI or vehicle was performed and then stimulated with LPS for 4 h. Differential gene expression confirmed that DI-treatment resulted in down-regulation of a spectrum of pro-inflammatory transcripts (see e.g., FIG. 1E), including Nos2, II6, and II12b, as a part of a global down-regulation of immune activation pathways (see e.g., FIG. 6).

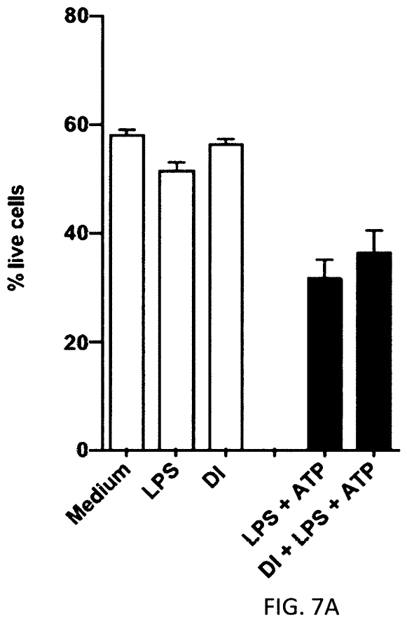

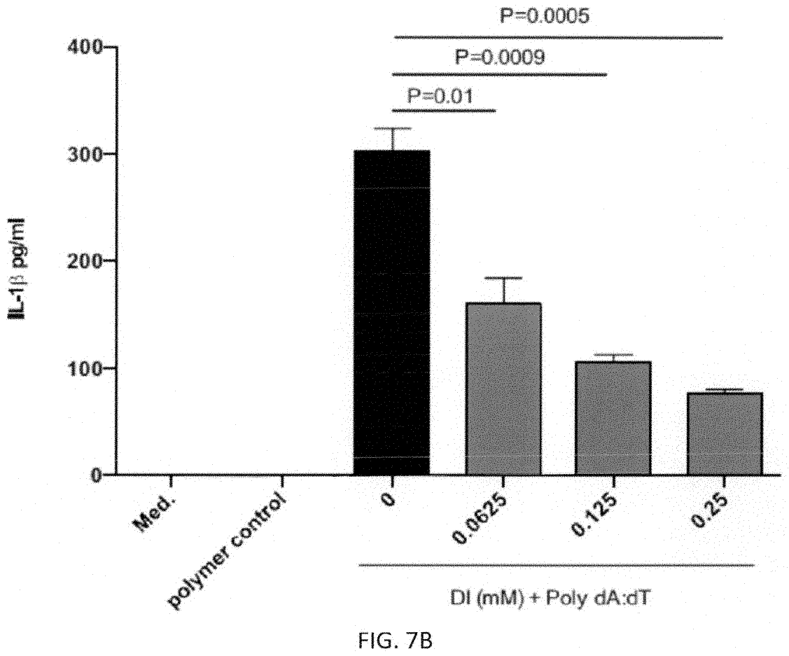

The RNA-seq analysis also revealed that pretreatment with DI modulated the expression of a number of LPS-regulated genes involved in inflammasome function (see e.g., FIG. 1E), including II1b, II18, P2rx7, Casp1, and the inflammasome adapter Pycard (ASC). Indeed, DI potently inhibited production of mature IL-1β and IL-18 induced under prototypical NLRP3-activating conditions, namely LPS-driven priming (signal 1) followed by signal 2-inducers ATP, nigericin, and monosodium urate crystals (see e.g., FIG. 1F, FIG. 1G), whereas it affected inflammasome-induced cytotoxicity only moderately (see e.g., FIG. 7A). DI-treated BMDM also had impaired IL-1β production following AIM-2-dependent inflammasome activation (see e.g., FIG. 7B), suggesting a broader regulatory effect on inflammasome activation. The decreased protein expression of pro-IL-1β, ASC and NLRP3 in DI-treated cells (see e.g., FIG. 1H) indicated that itaconate-mediated inhibition of inflammasome function was due primarily to a defective priming phase.

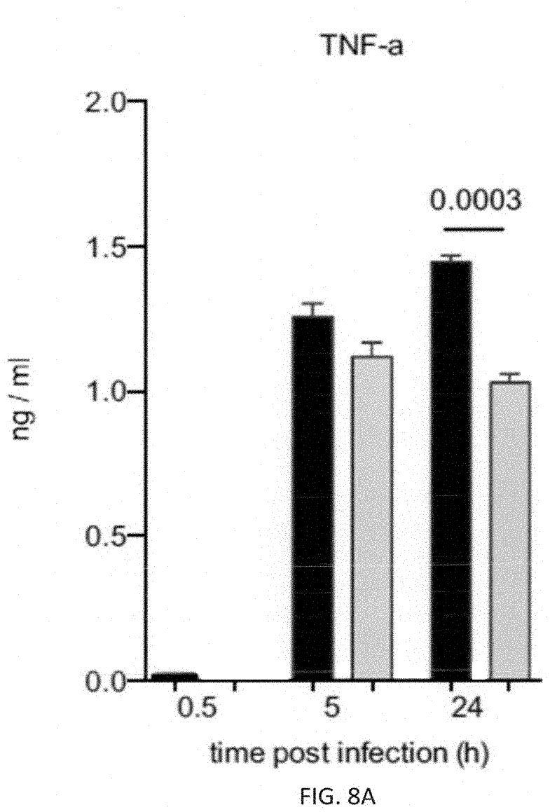

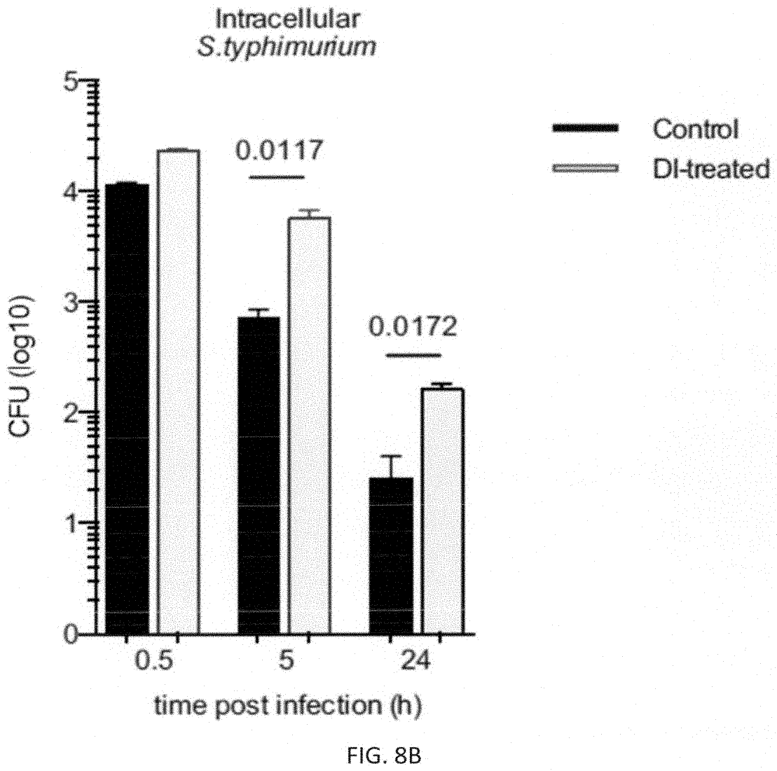

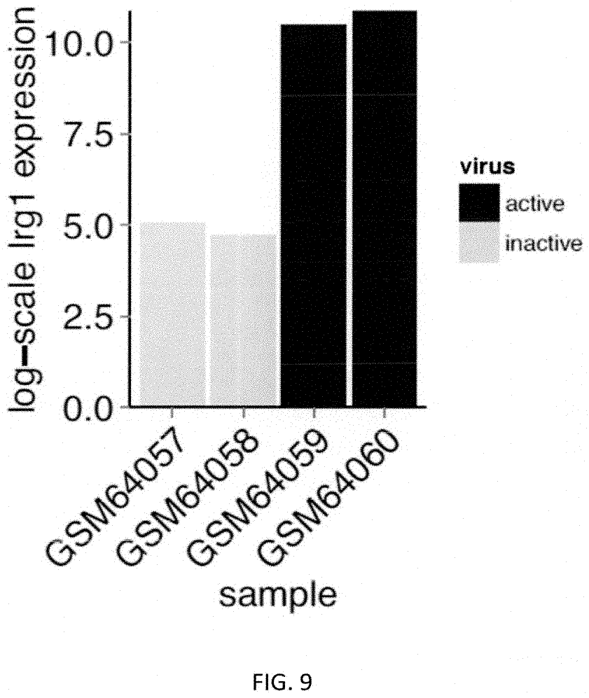

Bactericidal action of itaconate when added exogenously to live Salmonella enterica and Mycobacterium tuberculosis cultures has been shown. However, the effect of itaconate on bacteria in the context of infection of macrophages was not tested to date. To evaluate this, BMDMs were infected with Salmonella typhimurium, an intracellular gram-negative bacterium that triggers TLR4 signaling and NLRP3 inflammasome activation in the presence or absence of DI pretreatment. Without itaconate, infection of untreated BMDMs with S. typhimurium resulted in expected production of IL-1β, IL-6, and nitric oxide (NO) (see e.g., FIG. 1I). Remarkably, these cytokine responses were virtually abrogated in cells that were pre-treated with DI (see e.g., FIG. 1I), whereas TNF-α levels were only marginally affected (see e.g., FIG. 8A). Importantly, the number of intracellular bacteria was comparable between DI-treated and control BMDM (see e.g., FIG. 8B), indicating that the anti-inflammatory effects of itaconate did not result directly from bactericidal activity. Thus, besides its previously reported activity of inhibiting isocitrate lyase (Id), a bacterial glyoxylate shunt enzyme catalyzing the conversion of isocitrate to succinate and glyoxylate, an additional role of itaconate in the host cell response was observed. Furthermore, the fact that Irg1 is induced by viral as well as bacterial infections also points to itaconate having additional regulatory functions that are not specific to anti-bacterial response (see e.g., FIG. 9).

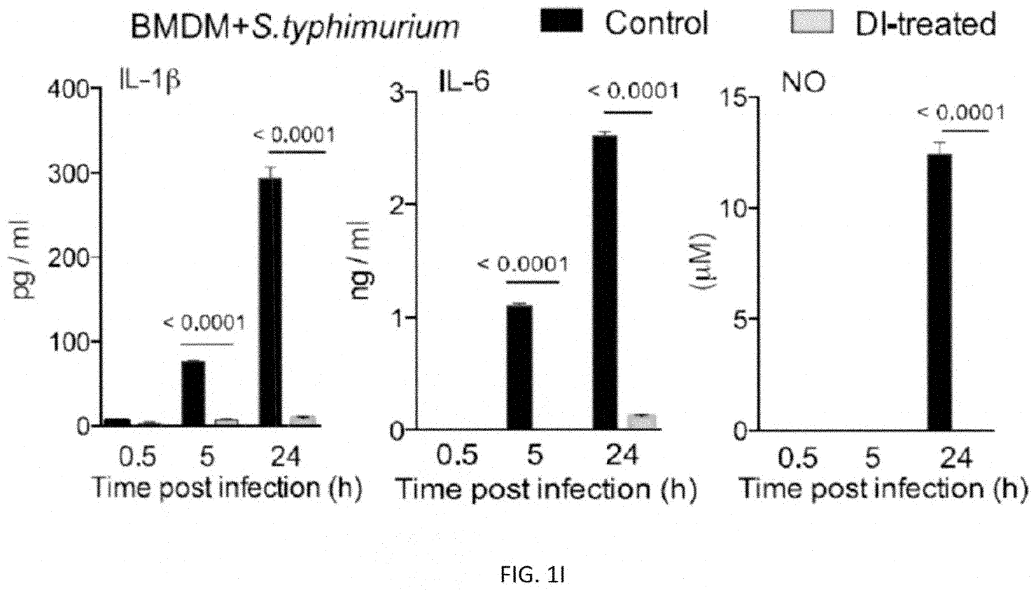

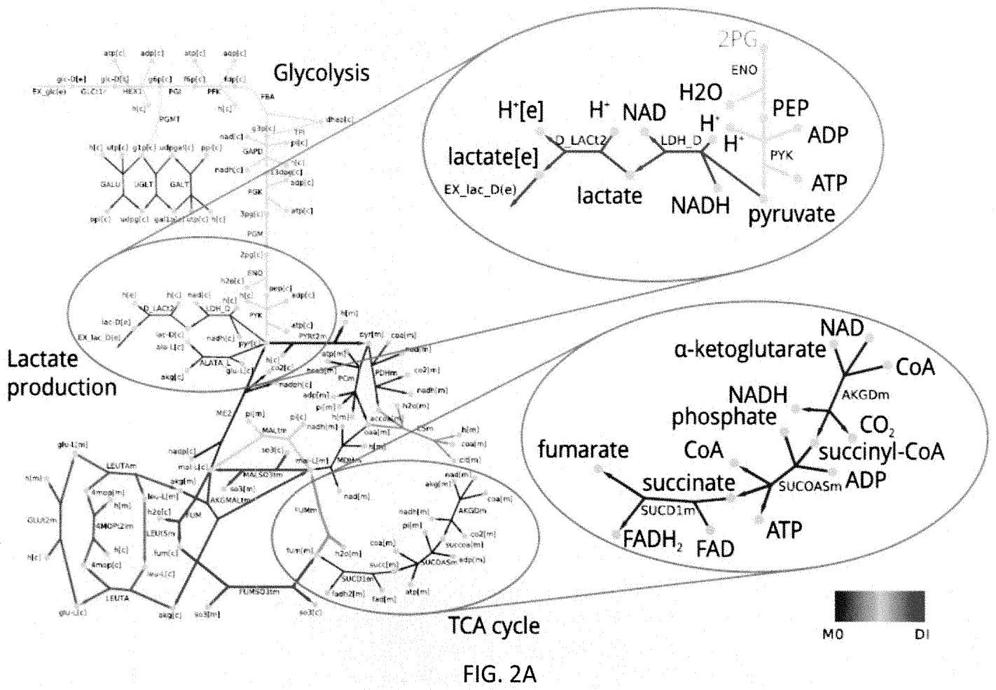

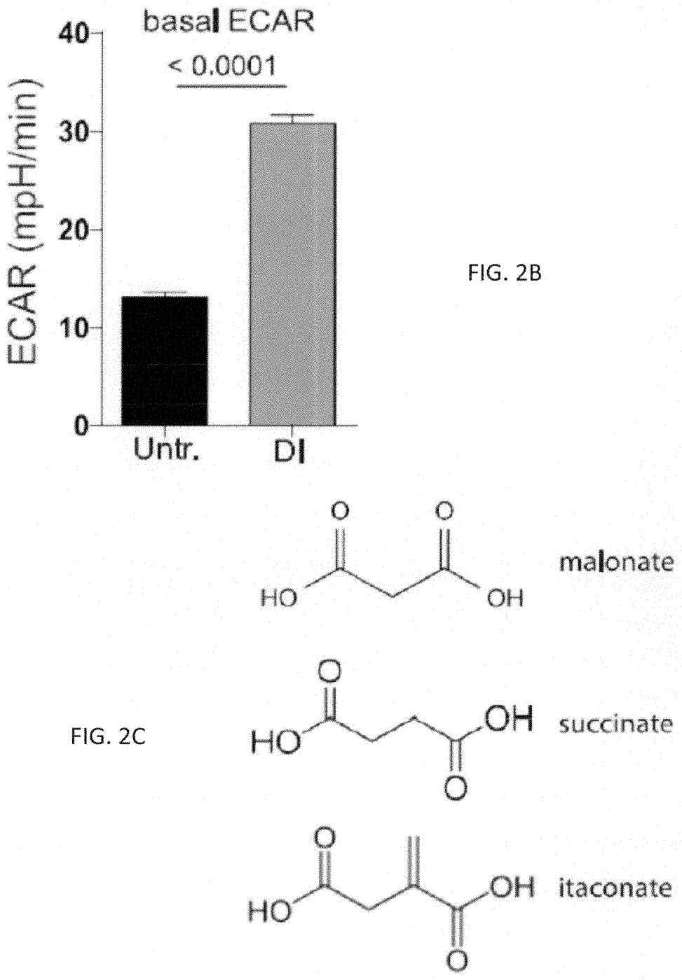

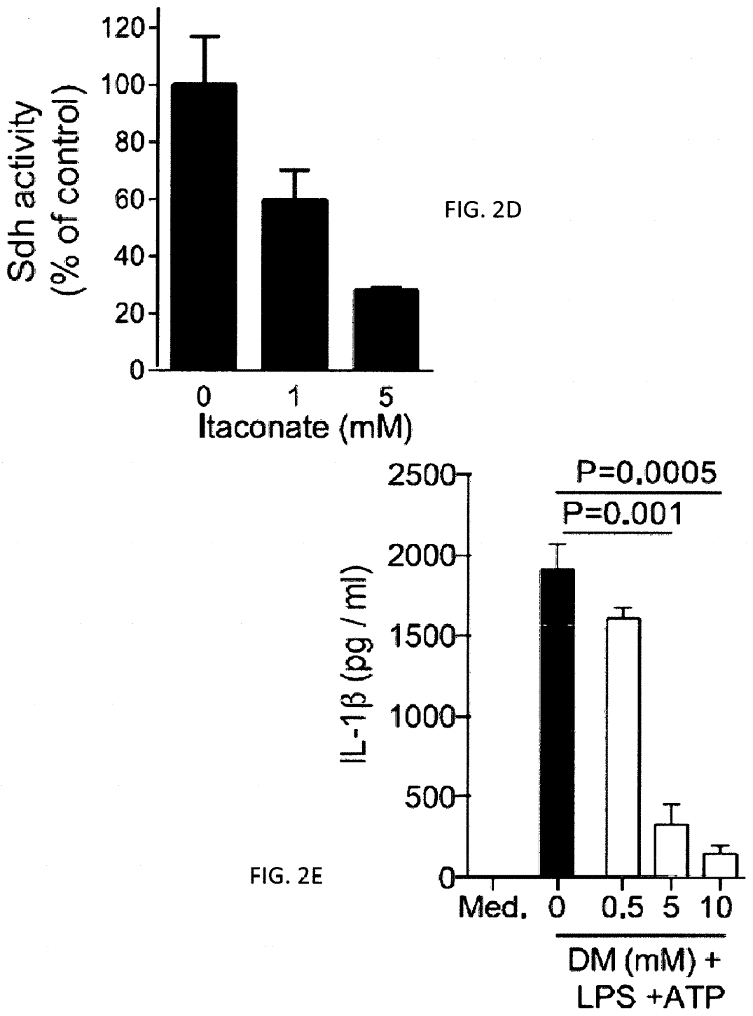

As perturbations in cellular metabolism can lead to transcriptional defects in IL-1β production and inflammasome activation, it was hypothesized that itaconate exerts its anti-inflammatory action, in part, by interfering with cellular metabolism. Using computational analysis of transcriptional data, the possible rewiring of the metabolic flux triggered by itaconate in the absence of LPS was investigated. Using a flux balance analysis framework, the metabolic model originally formulated for RAW 264.7 macrophage cell line was extended by including several reactions and enzymes that were absent in the original model (e.g. Irg1 and itaconate, see Methods for details). The fluxes in untreated and itaconate-treated conditions that were most consistent with the RNA-seq data were searched (see e.g., Methods in Example 1). A comparative network highlights three types of metabolic flux change in response to itaconate treatment (see e.g., FIG. 2A, FIG. 10 for detailed network): decreased metabolic flux (blue edges), increased metabolic flux (red) and reactions insensitive to itaconate addition (grey).

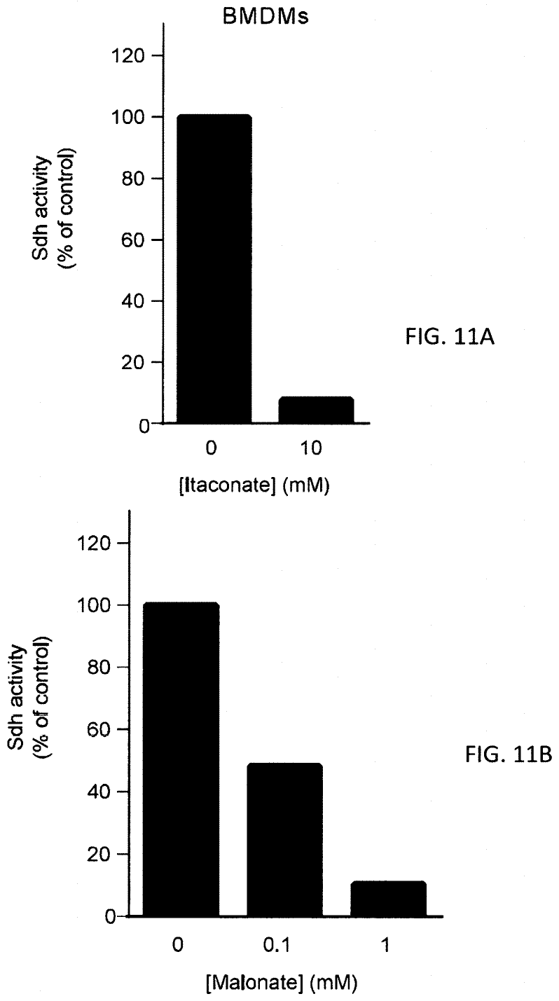

Two features of the computational analysis were apparent. First, itaconate addition was predicted to increase lactate dehydrogenase (Ldh) production. To confirm this experimentally, a metabolic SeaHorse analysis of unstimulated BMDM with and without DI treatment was performed. It was observed that extracellular acidification rate (ECAR), which occurs as a consequence of lactate accumulation in the medium, was increased in the presence of itaconate (see e.g., FIG. 2B). The second computational prediction was that itaconate addition should decrease the metabolic flux through succinate dehydrogenase (Sdh). This suggested that itaconate competitively inhibits Sdh, conceivably due the structural similarity between itaconate, succinate, and the Sdh inhibitor, malonate (see e.g., FIG. 2C). To evaluate this hypothesis directly, the activity of purified Sdh in the presence or absence of itaconate was compared. Notably, itaconate blocked the activity of Sdh in a dose-dependent manner (see e.g., FIG. 2D and FIG. 11A), as did malonate (see e.g., FIG. 11B). This data suggests that the anti-inflammatory effects of itaconate in BMDM likely were due to inhibition of Sdh. In support of this notion, pre-treatment of BMDMs with dimethyl malonate also inhibited IL-18 production after LPS+ATP stimulation (see e.g., FIG. 2E).

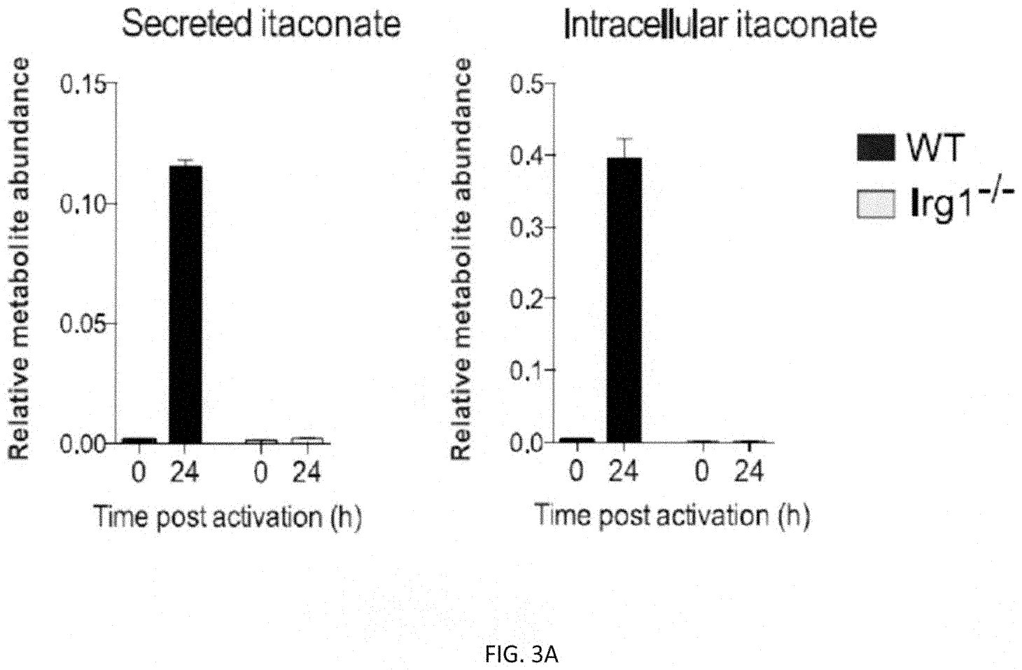

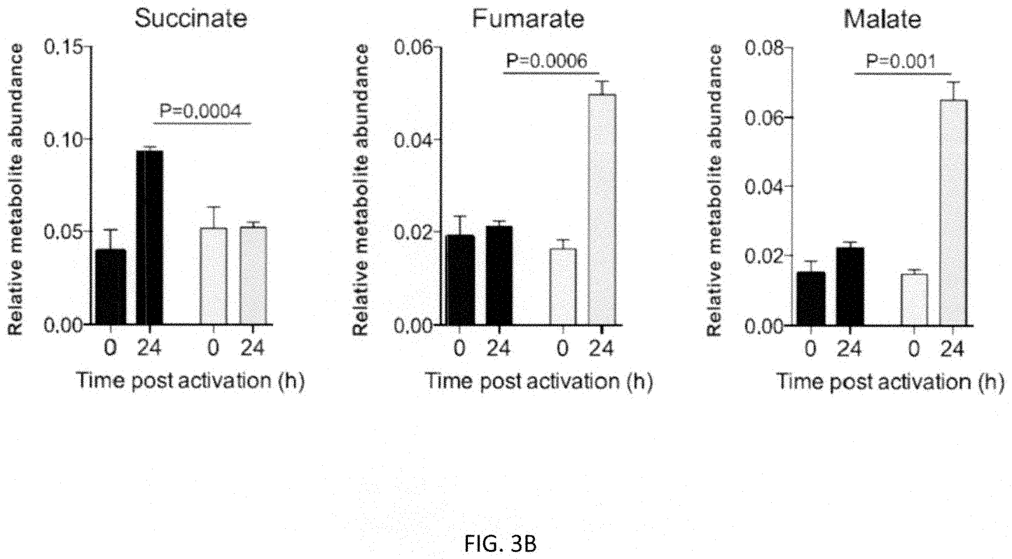

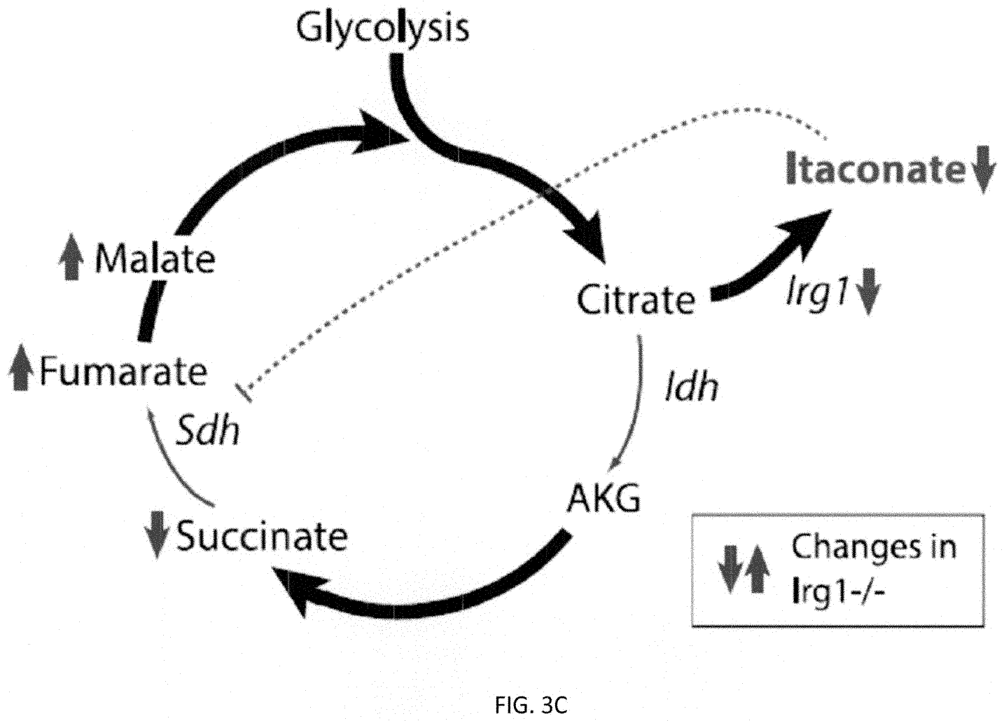

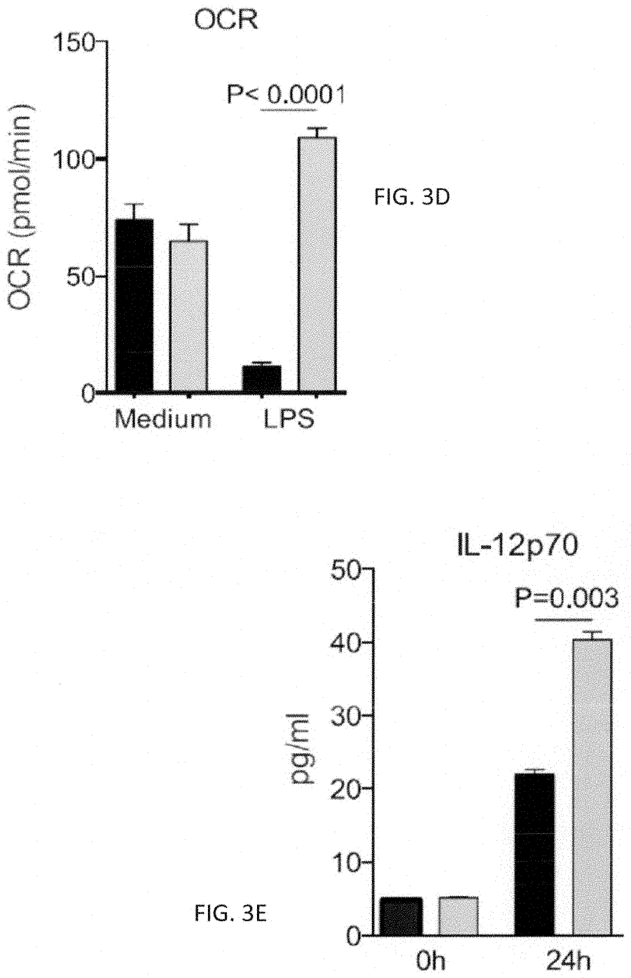

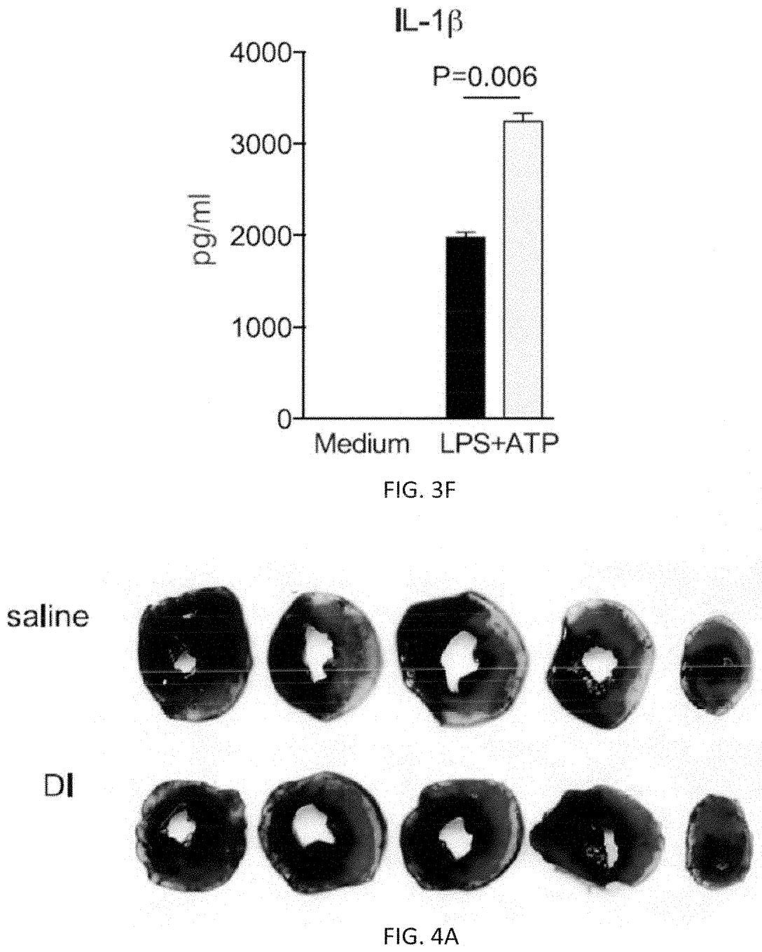

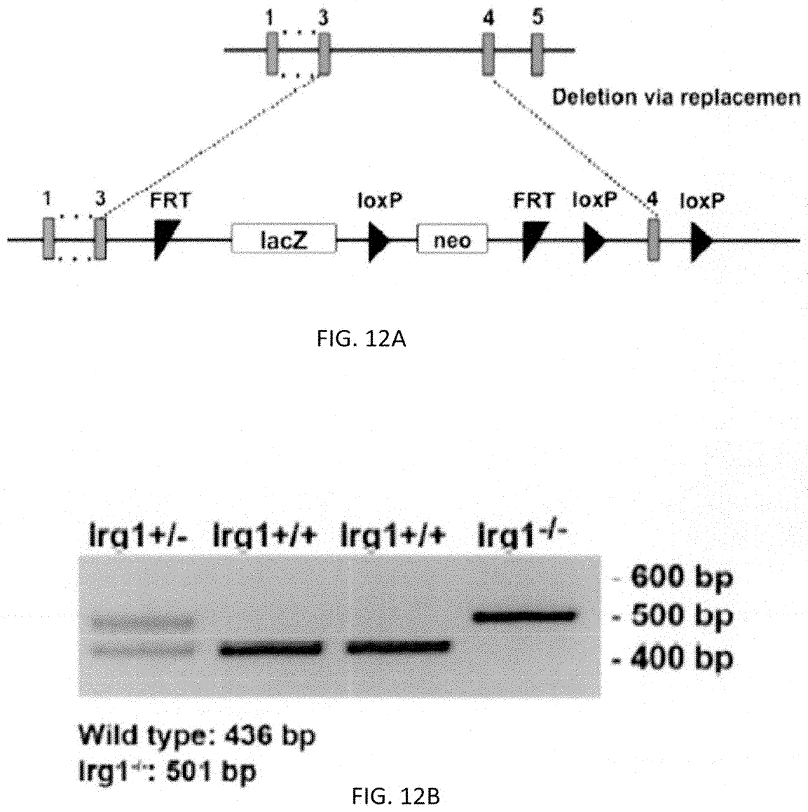

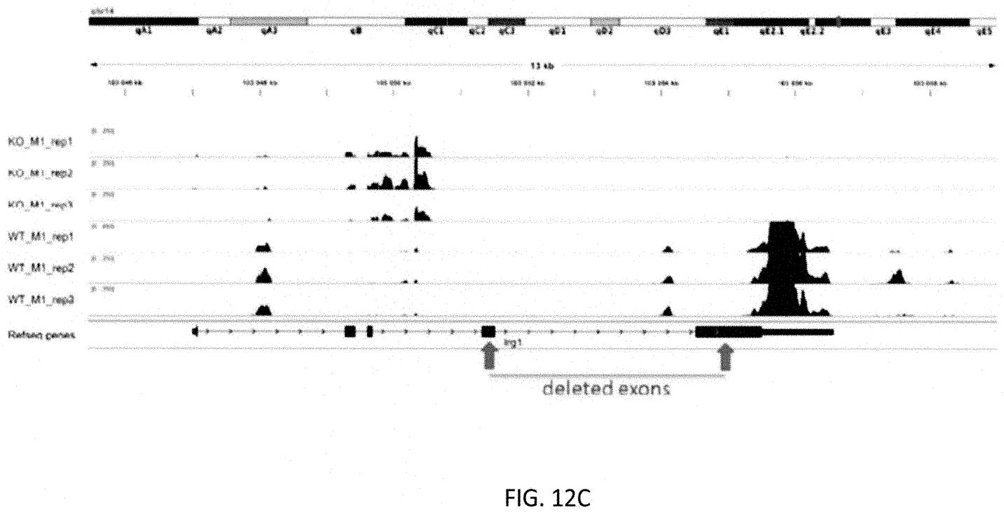

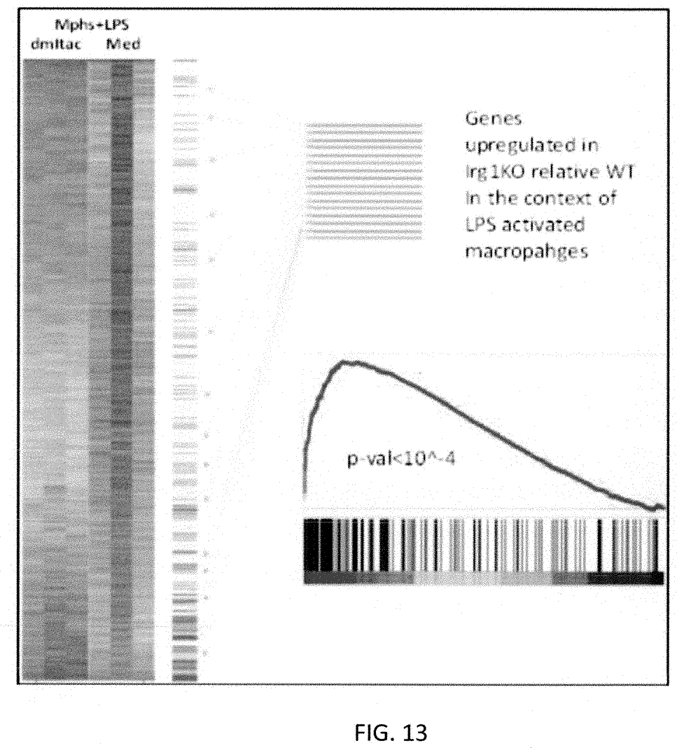

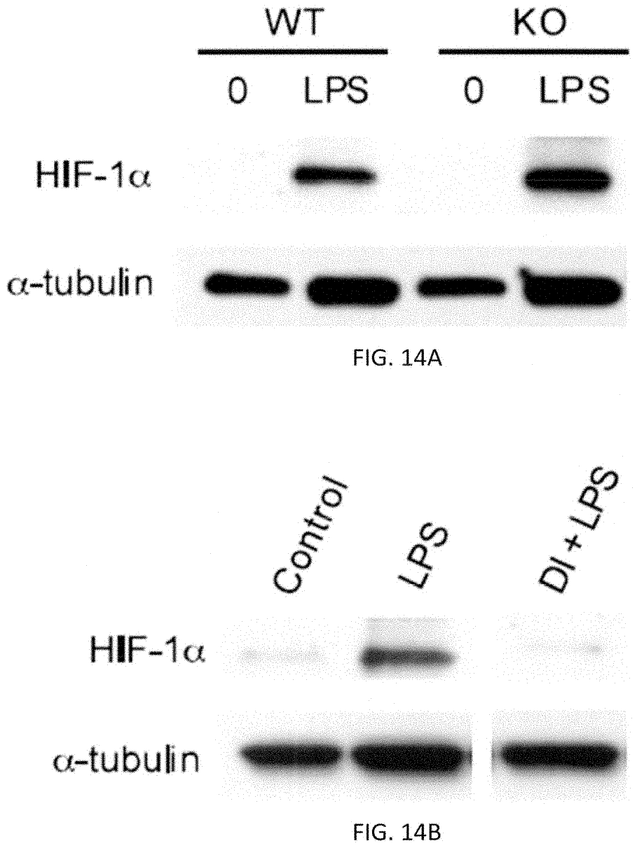

Collectively, these observations suggested a key role for itaconate in macrophage metabolism and effector functions. To test the physiological relevance of this hypothesis, mice with a targeted disruption of the Irg1 gene were generated (see e.g., FIG. 12); Irg1 has been reported as the enzyme responsible for synthesis of itaconate in inflammatory macrophages. As BMDMs from Irg1^{-/-} mice failed to produce or secrete itaconate (see e.g., FIG. 3A) after stimulation with LPS and IFN-γ, it was concluded and presently believed that Irg1 is the only enzyme carrying out itaconate synthesis under these conditions. Next, RNA-seq was used to profile differences in gene expression of LPS-activated wild-type (WT) and Irg1^{-/-} BMDMs. Notably, the transcriptional signature in Irg1^{-/-} cells essentially was the inverse of that from itaconate-treated WT cells: genes upregulated in Irg1^{-/-} cells were downregulated in itaconate-treated WT BMDMs (see e.g., FIG. 13), suggesting that endogenous itaconate functions in a manner similar to that described for exogenously added DI. To validate this assumption, the metabolites of Irg1^{-/-} BMDMs was profiled, which revealed changes indicative of altered Sdh activity. A lack of Irg1 expression resulted in reduced levels succinate, and a corresponding accumulation of fumarate and malate (see e.g., FIG. 3B). Thus, in the absence of itaconate, Sdh remained active and oxidized succinate to fumarate, which was rapidly converted to malate (see e.g., FIG. 3C). Besides its role in the TCA cycle, Sdh (termed complex II in this context) also is part of the mitochondrial electron transport system and catalyzes the release of electrons from succinate that feed the ubiquinone cycle, which is necessary for mitochondrial respiration. In a natural course of inflammatory activation of macrophages, mitochondrial respiration is decreasing significantly, potentially due to reduced Sdh activity. Thus, itaconate-mediated inhibition of Sdh influenced mitochondrial function was tested by measuring oxygen consumption rates (OCR). Remarkably, relative to WT cells, BMDMs did not show the reduced OCR at 24 h after activation (see e.g., FIG. 3D), indicating that inhibition of Sdh activity by endogenous itaconate contributes significantly to the regulation of mitochondrial respiration in inflammatory macrophages. Collectively, these results confirmed the function of itaconate as a physiological regulator of Sdh activity in inflammatory macrophages. Accordingly, Irg1^{-/-} BMDM produced more IL-12 in response to LPS and IFN-γ compared to WT cells (see e.g., FIG. 3E) and sustained higher expression of mature IL-1β under conditions that stimulate NLPR3 (see e.g., FIG. 3F). Consistent with the known IL-1β-promoting effect of HIF-1α, increased HIF-1α protein levels were observed in cells and reciprocally, suppression of HIF-1α expression in itaconate-treated BMDM (see e.g., FIG. 14).

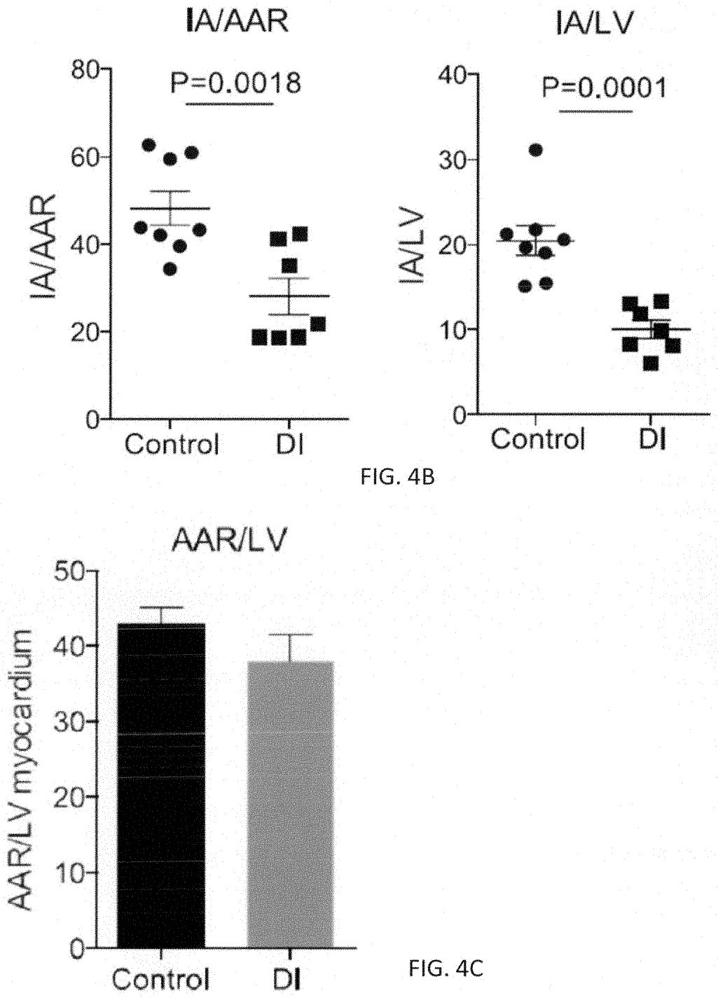

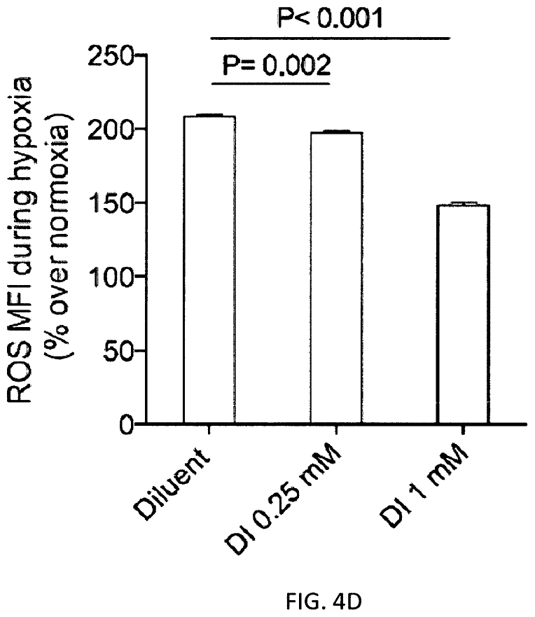

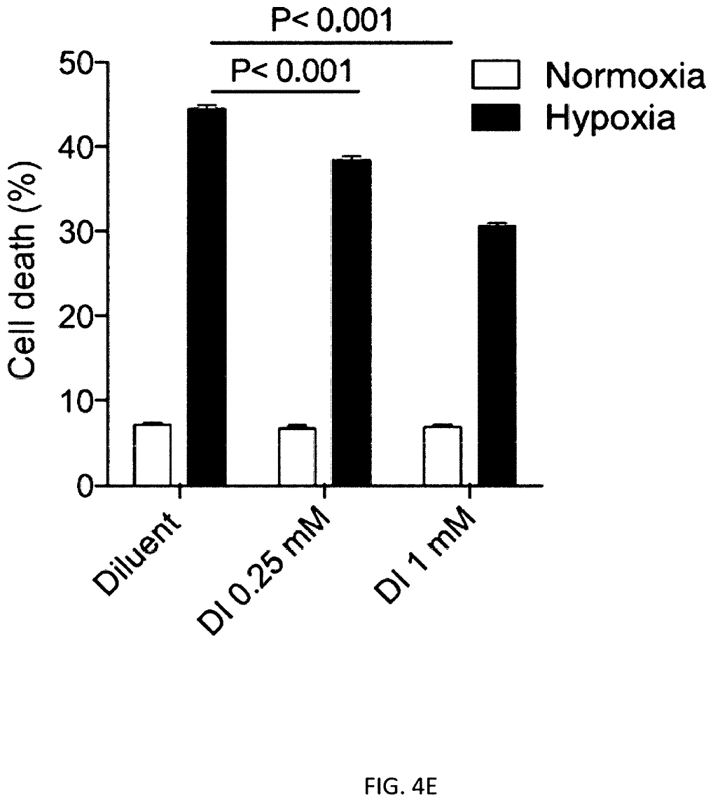

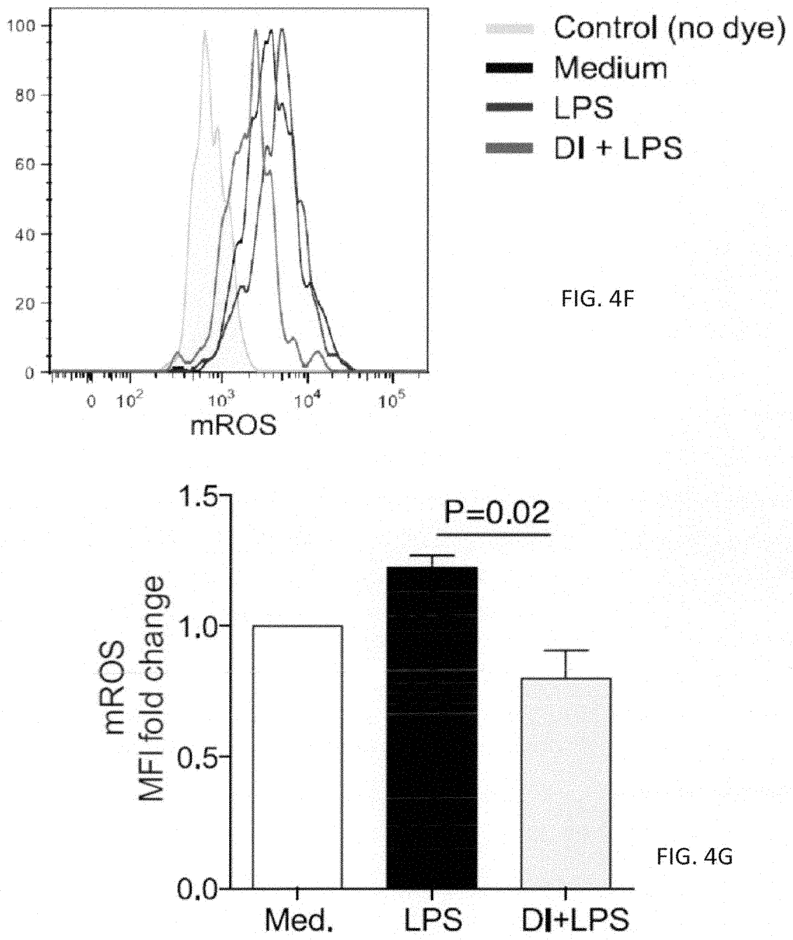

It was discovered that intravenous infusion of DI during ischemia markedly reduced myocardial infarct size (see e.g., FIG. 4A, FIG. 4B). While the area-at-risk was similar between the two groups (see e.g., FIG. 4C), the 42% reduction in infarct size with DI treatment was comparable to that seen following dimethyl malonate administration. It was thought that the cardioprotective effects of DI might involve the regulation of mROS production. To evaluate this hypothesis, an in vitro assay that mimics or models myocardial infarction injury by subjecting neonatal cardiomyocytes to hypoxic insult was used. Pretreatment with DI attenuated the hypoxia-induced increase in ROS generation (see e.g., FIG. 4D) and conferred dose-dependent protection from hypoxia-induced cell death (see e.g., FIG. 4E).

In the context of macrophage activation, mROS production appears to be driven by the reverse flow of the electron transport chain as it is inhibited by the mitochondrial complex I inhibitor rotenone. Accordingly, it was tested if blockade of Sdh activity with itaconate affected mROS generation. Pre-treatment with DI impaired the ability of BMDM to upregulate mROS in response to LPS (see e.g., FIG. 4F, FIG. 4G). As interfering with mROS generation is known to affect inflammasome priming, the blunted mROS response provides a mechanistic link between Sdh inhibition and the anti-inflammatory effects of itaconate on IL-1β and IL-18 production (see e.g., FIG. 1F).

Overall, this work identifies a posttranscriptional regulatory mechanism that governs TCA cycle remodeling and, consequently, macrophage activation via inhibitory effect of itaconate on Sdh. It was shown that itaconate is an inhibitor of the Sdh activity during macrophage activation. In this model, itaconate functions as a “release valve” on the TCA cycle, providing a mechanism to regulate succinate conversion to fumarate. This effectively allows itaconate to dampen mROS production, and prevents the excessive inflammatory cytokine production that leads to tissue damage. The present results expand the physiological roles of itaconate, previously thought to be restricted to direct anti-bacterial action via inhibition of Id, to include regulation of TLR-mediated inflammatory cytokine production, and provides a physiological regulatory mechanism to control electron transport chain flow, succinate levels, ROS production, and tissue inflammation.

(I) Methods for Example 1.

Mice.

Irg1^{-/-} (MGI:103206) mice were generated at Washington University after receiving embryonic stem (ES) cells (Irg1^{tm1a(KOMP)Wtsi}) from the Knockout Mouse Project Repository (KOMP, University of California, Davis) containing an insertion cassette between exons 3 and 4. This cassette prevents transcription of downstream exons 4 and 5 and production of mature protein. Irg1^{-/-} C57BL/6N ES cells were microinjected into (Cg)-Tyrc-2J/J albino recipient female C57BL/6 mouse blastocysts. Chimeric mice with black coat color were selected and bred to wild type C57BL/6N mice. Homozygous mice Irg1^{-/-} were generated by intercrossing the heterozygous animals and confirmed by PCR. mice were fertile and exhibited normal Mendelian frequencies and BMDM from both sexes were used.

C57BL/6N WT mice were purchased from Charles Rivers Laboratories and used as age-matched controls. Mice were maintained at Washington University under specific pathogen-free conditions in accordance with Federal and University guidelines and protocols approved by the Animal Studies Committee of Washington University.

Differentiation and Activation of Macrophages.

BMDM were prepared from 6 to 8 week-old mice as previously described and seeded at 10⁵ cells/well in 96 well tissue-culture plates in RMPI-1640 medium (Gibco) supplemented with 10% FBS, 2 mM L-glutamine (Gibco) and 100 U/ml penicillin-streptomycin (Gibco). For in vitro bacteria infection experiments, kanamycin was added instead of penicillin-streptomycin. Cells were treated or not with DI (Sigma), and then stimulated as indicated with LPS (Escherichia coli serotype 0111:B4; Sigma), ATP (Sigma), nigericin (Sigma), monosodium urate crystals (MSU) (InvivoGen). For activation of the AIM2-inflammasome, untreated of DI-treated BMDM were transfected with poly (dA:dT) (InvivoGen) complexed to Xfect polymer (Clontech laboratories) as previously described; as a control, some cells received polymer alone.

Flow Cytometry.

Cells were incubated with FcγR-blocking anti-CD16/32 (clone 93, Biolegend) before staining with antibodies to F4/80 (clone BM8, eBioscience) and CD11b (clone M1/70; BD Pharmingen) together with LIVE/DEAD dye (Invitrogen). Intracellular iNOS was detected with mouse anti-NOS2 (clone C-11, Santa Cruz Biotechnology) followed by incubation with fluorochrome-conjugated anti-mouse IgG (Clone A85-1, BD Pharmingen) using the BD cytofix/cytoperm Kit (BD Biosciences) according to manufacturer's instructions. Cells were acquired on a Canto II flow cytometer (BD Biosciences) and data were analyzed using FlowJo v.9.5.2 software (Tree Star).

Quantification of Cytokines and Nitric Oxide in Culture Supernatants.

The concentration of cytokines in culture supernatants was determined using the following kits according to manufacturer's instructions. DuoSet® kits for mouse IL-1β/IL-1F2, TNF-α and IL-6 (all R&D systems); mouse IL-12 ELISA MAX™ Deluxe Set (BioLegend); mouse IL-18 ELISA kit (MBL). Nitric oxide was quantified using Griess Reagent System (Promega).

Western Blotting.

BMDM were stimulated as indicated, washed with PBS, lysed in RIPA Lysis Buffer System (Santa Cruz) and boiled in sample loading buffer containing SDS and 100 mM DTT. Proteins were separated by electrophoresis through 4-20% polyacrylamide gradient gels (BioRad). After electrophoretic transfer of proteins onto PVDF membranes, nonspecific binding was blocked by incubation with 5% skim milk and membranes were probed with primary antibodies anti-IL1β (1:1000 dilution; 12507S, CellSignaling), NIrp3 (1:500 dilution; NBP2-12446, Novus), HIF-1α (1:500 dilution; NB100-449, Novus), α-tubulin (1:2000 dilution; 2125S, CellSignaling), ASC (1:1000 dilution; sc-22514-R, Santa Cruz) followed by incubation with secondary antibodies (horseradish peroxidase-conjugated anti-rabbit, 1:10,000 dilution; sc-2030, Santa Cruz) and visualized with Clarity™ western ECL substrate (Biorad).

Sdh Activity Assay.

Sdh was purified from BV2 cells (a macrophage cell line) or BMDM and its activity was measured in the presence of itaconate or malonate using the Complex II Enzyme Activity Microplate Assay Kit (Abcam)-based on a colorimetric detection method according to the manufacturer's protocol. The test compounds were diluted in activity buffer and added to the phospholipid mixture 5 min before adding the activity solution. Absorbance was measured at 600 nm.

In Vitro Infection with Salmonella typhimurium.

This in vitro infection protocol was adapted from Weiss et al. Twenty-four-well plates were seeded with 2×10⁵ BMDM/well in 500 μl of RPMI-1640 medium (Gibco) supplemented with 10% FBS (Hyclone), 2 mM L-glutamine (Sigma) and 100 μg/ml kanamycin (Sigma). Cultures were treated with 0.25 mM DI (final concentration) or vehicle (medium). After 12 h, 200,000 CFU S. Typhimurium (strain SB100; kanamycin resistant) were added (time zero) and plates were centrifuged for 10 min at 850×g. Gentamicin (100 μg/ml; Sigma) was added at 30 min after infection. Triplicate samples were plated for each time point: 30 min, 6 h and 24 h. To determine the number of intracellular bacteria, supernatants was removed and cells were lysed by adding 50 μl of 10% Triton X-100 for 10 min before addition of 450 μl of cold sterile PBS. Appropriate dilutions were made, and samples were plated on Luria agar plates containing 10 μg/ml kanamycin (Sigma). Colonies were counted the next day.

Extracellular Flux Analysis.

Extracellular acidification rate (ECAR) and Oxygen Consumption rate (OCR) were measured in real time using Seahorse technology as described previously.

Metabolite Profiling by GC-MS.

Cellular metabolites were extracted from equal numbers of cells for each sample and analyzed by GC-MS as previously described Briefly, intracellular metabolism was quenched by the addition of 800 μl of 80% methanol. To analyze secreted metabolites, 10 μl of cell culture media was added to 800 μl of 80% methanol. D-myristic acid (750 ng/sample) was added as an internal standard to all metabolite extracts. Extracts were dried by vacuum centrifuge and pellets were resuspended in 30 μl of pyridine containing 10 mg/ml methoxyamine hydrochloride, before being derivatized using N-(tert-butyldimethylsilyl)-N-methyltrifluoroacetamide. Metabolite abundance was expressed relative to the internal standard.

RNA Seq Analysis.

mRNA was extracted from cell lysates by means of oligo-dT beads (Invitrogen). For cDNA synthesis, a custom oligo-dT primer with a barcode and adaptor-linker sequence (CCTACACGACGCTCTTCCGATCT-XXXXXXXX-T15) (SEQ ID NO: 1) was used. After first-strand synthesis, samples were pooled together based on Actb qPCR values and RNA-DNA hybrids were degraded with consecutive acid-alkali treatment. Subsequently, a second sequencing linker (AGATCGGAAGAGCACACGTCTG) (SEQ ID NO: 2) was ligated with T4 ligase (NEB) followed by SPRI-beads (Agencourt AMPure XP, BeckmanCoulter) clean-up. The mixture was enriched by PCR for 12 cycles and SPRI-beads (Agencourt AMPure XP, BeckmanCoulter) purified to yield final strand-specific RNA-seq libraries. Libraries were sequenced using a HiSeq 2500 (Illumina) using 50 bp×25 bp pair-end sequencing. Second read (read-mate) was used for sample demultiplexing. Reads were aligned to the GRCm38.p2 assembly of mouse genome using STAR aligner. Aligned reads were quantified using quant3p script (github.com/ctlab/quant3p) to account for specifics of 3′ sequencing. RefSeq genome annotation was used and DESeq2 was used for differential gene expression analysis.

Flux Balance Analysis.

To investigate possible rewiring of the metabolic fluxes, a flux balance analysis framework (FBA) was used. Using the RAW 264.7 macrophage cell line metabolic model and an algorithm similar to GIMME and MADE, the fluxes in untreated and itaconate-treated conditions were simulated based on their consistency with the obtained RNA-seq data for each condition. First, RAW 264.7 model was updated as follows: i) added reactions regulated by Irg1, Aco1 and Aco2 and itaconate transport reactions; ii) updated several reaction-gene associations by adding missing homologs for the genes in corresponding Recon 2 reactions; iii) removed dependence of some OXPHOS reactions on the large gene complexes due to insufficient gene annotation; iv) removed octadecanoate (n-C18:0 and n-C18:1) and tetradecanoate (n-C14:0) production reaction from nothing and v) added ATP, NADH, AcCoA usage reactions. (ATP+H2O→ADP+H⁺+PO3, NADH→NAD+H⁺, AcCoA→CoA). Next, the maximal possible biomass production rate was found by running FBA linear optimization using the sybil R package. A biomass production of at least 50% of this rate was added as a constraint to all consequently used models. For each reaction and each of the two conditions (untreated and Itaconate-treated) an inconsistency penalty per unit of additional flux was calculated. Following the MADE algorithm (Jensen et al, 2011), differential expression data was used as an indicator of a gene (and a corresponding reaction) being inactive: a down-regulated gene (p-value<1 e-2) in a given condition (untreated or Itaconate-treated) compared to any other condition (unstimulated macrophages, or with addition of LPS, itaconate, itaconate+LPS) was considered inactive. More formally, a penalty for a gene was calculated as min(−log 10(p)+2, 0), where p is the gene minimal p-value among all three comparisons. The reaction penalty scores per unit of flux were calculated by substitution into the corresponding gene-reaction rule “min” and “max” operations instead of “or” and “and” operations and gene penalty instead of a gene. Next, a linear optimization was ran to find the fluxes for untreated and Itaconate-treated conditions with the minimal total inconsistency score and that produced biomass with at least 50% of the optimal rate. Because a reaction inconsistency score depends on the absolute flux, to calculate it each reversible reaction was split into two irreversible reactions corresponding to the forward and reverse directions. In this way, the total flux inconsistency was calculated as a sum of fluxes through individual reactions multiplied by the corresponding penalty score per unit of flux. Also, a minimal total flux assumption was used and thus added 0.001 penalty per unit of flux for each reaction. To compare fluxes, reactions that had reaction rates greater than 0.1 unit in any of the two conditions were selected and selected the biggest connected component, ignoring ubiquitous metabolites such as water or ATP. These reactions are shown on the FIG. 2A. The width of an edge is proportional to sum of fluxes in M0 and M0+ itaconate conditions with a color displaying relative difference of the fluxes in the conditions. Blue edges correspond to reactions that have flux only in M0 condition and red edges correspond to reactions that have flux only in M0+Itac conditions. Reactions that have equal predicted fluxes are shown in grey. For the visualization purposes akg[c], akg[m] and glu-L[c] vertices were split in two.

Myocardial Ischemia-Reperfusion Model.

In vivo ischemia-reperfusion modeling was performed as previously described. 8 to 10 week-old mice were anesthetized and subjected to an open chest procedure of reversible left anterior descending artery ligation for 30 min and subsequent reperfusion for 2 hours. Saline or DI (4 mg/Kg/min) was infused intravenously for 10 min before and throughout the ischemic period. A cardioplegic solution followed by TTC at 37° C. and then Evans Blue (after reocclusion of the LAD) was injected in a retrograde manner through the aorta in situ. The left ventricle was then sectioned into 5 slices and image analysis was performed with Image J (NIH).

Hypoxia Modeling in Neonatal Rat Cardiac Myocytes.

Neonatal rat cardiac myocytes were isolated and cultured as described. Cells were subjected to hypoxia in an oxygen control cabinet (Coy Laboratories, Grass Lake, Mich.) mounted within an incubator and equipped with oxygen controller and sensor for continuous oxygen level monitoring. A mixture of 95% nitrogen and 5% CO2 was utilized to create hypoxia, and oxygen levels in the chamber were monitored and maintained at <1%. Cell death was assessed with the Live-Dead Cytotoxicity Viability kit for Mammalian cells (Invitrogen) and ROS generation was monitored by flow cytometric assessment of carboxy-H2DCFDA fluorescence (following incubation in 10 μmol/L for 30 minutes), as previously described.

Measurement of mROS.

Cells were incubated with DI (0.25 mM, 12 h) or vehicle and then loaded with 5 μM of the mitochondrial superoxide indicator MitoSOX (Invitrogen) at 37° C. for 30 min in HBSS supplemented with 0.1% BSA. Cells were then rinsed with warm culture medium and LPS treatment was started 1 h later. Cells were harvested and analyzed on a Canto II flow cytometer (Becton-Dickinson).

### Example 2: Regulatory Role of Itaconate and Determine its Functional Impact on Immune Responses In Vivo Using Newly Generated Irg1^{-/-} and Irg1^{FL/FL} Mice

Metabolic rewiring is thought to be an important regulatory mechanism controlling activation of the immune cells. Succinate is thought to be one of the major metabolic regulators of macrophage activation, playing distinct proinflammatory role. As shown herein, data suggest that high production of itaconate during macrophage activation is functionally critical, as it provides an endogenous, structural mimetic metabolic regulator to balance the pro-inflammatory function of succinate.

As described herein, it was discovered that itaconate is one of the metabolites most highly produced extracellularly upon macrophage activation, with corresponding itaconate-producing enzyme (Irg1) levels increasing by >200 fold, putting Irg1 and itaconate on the same scale as other well-described metabo-transcriptional pairs: iNOS/NO, and Cox2/PGE2. Functionally, itaconate can have an anti-bacterial role through its inhibition of bacteria-specific enzyme isocitrate lyase (Icl). However, given the magnitude of itaconate production by activated macrophages, it was hypothesized that, analogous to NO and PGE2, itaconate may have immunomodulatory effects in addition to its antimicrobial effects. Indeed, by pre-treating macrophages in vitro with itaconate, significant reduction in the production of NO, IL-6, IL-1β was observed in response to LPS stimulation, as well as dose-dependent inhibition of IL-1β and IL-18 production during inflammasome activation. These effects are opposite to those previously described for succinate, in spite of the structural similarity of two compounds. In fact, structural mimetics of succinate (e.g. malonate, which is not found in immune cells naturally) are known to serve as inhibitors of succinate dehydrogenase (Sdh), an enzyme complex also known as complex II in the electron transfer chain (ETC). Indeed, the data show that mtROS-mediated injury, generated in the in vivo model of ischaemia-reperfusion model, is decreased significantly by addition of i.v. itaconate. Collectively, these observations suggest that the high production of itaconate during macrophage activation is functionally critical, as it provides an endogenous, structural mimetic metabolic regulator to balance the pro-inflammatory function of succinate. Here, the goal is to decipher the regulatory role of itaconate and determine its functional impact on immune responses in vivo using newly generated Irg1^{-/-} and Irg1^{fl/fl} mice that lack itaconate producing enzyme.

### Example 3: Confirm Itaconate as an Endogenous Inhibitor of Succinate Dehydrogenase (Sdh) that Provides a Succinate-Specific Break Point in the TCA Cycle and Regulation of ROS Production

To show itaconate is a competitive inhibitor of Sdh, a) classical biochemical assays are used to confirm its inhibitory properties; b) SeaHorse Metabolic assays to confirm change in respiratory function of cells upon addition of itaconate or itaconate+succinate mixtures are used; c) confirm that itaconate regulates succinate accumulation during inflammatory activation of macrophages by comparing succinate levels on WT and background; d) evaluate classical functional consequence of inhibiting ETC—modulation of ROS production—by measuring levels of mtROS in the cells upon different treatments and genetic backgrounds.

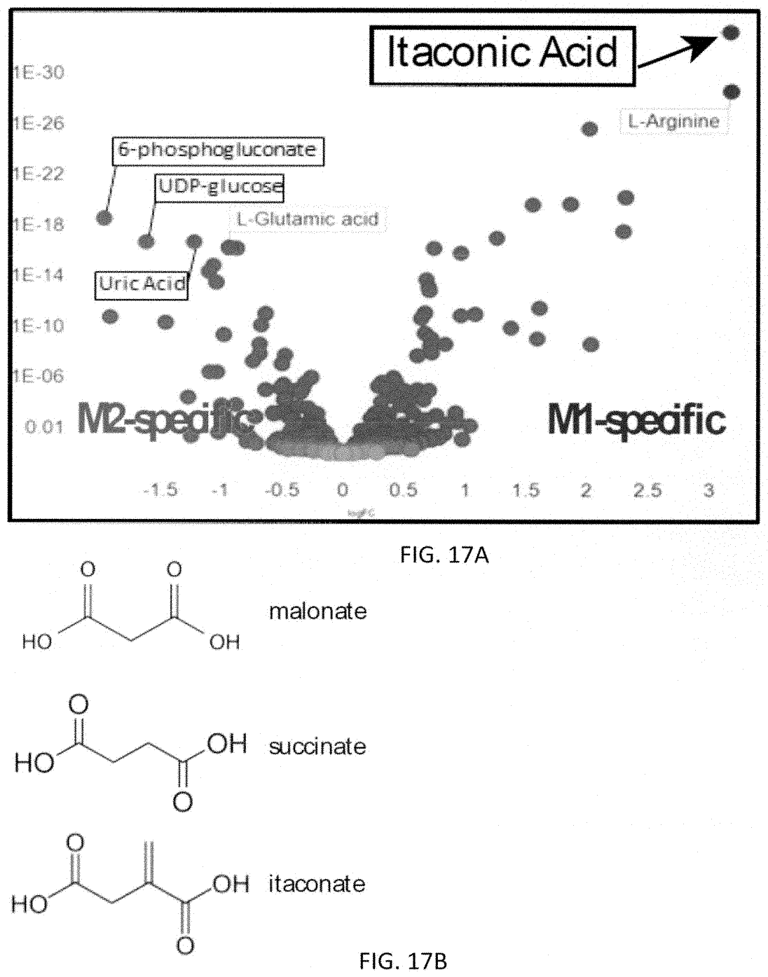

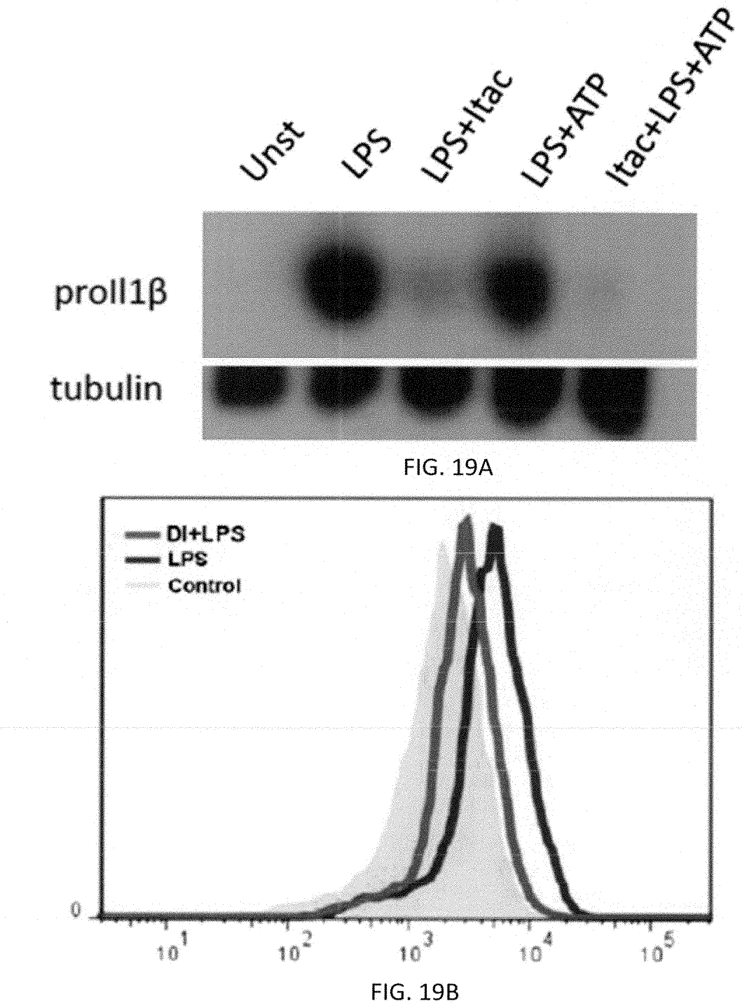

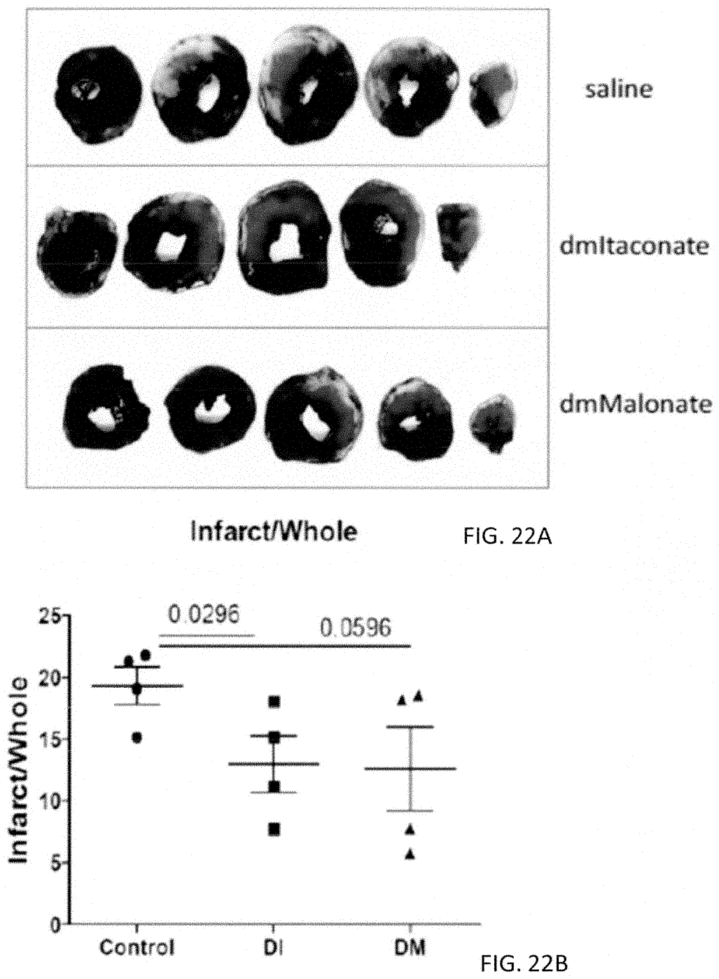

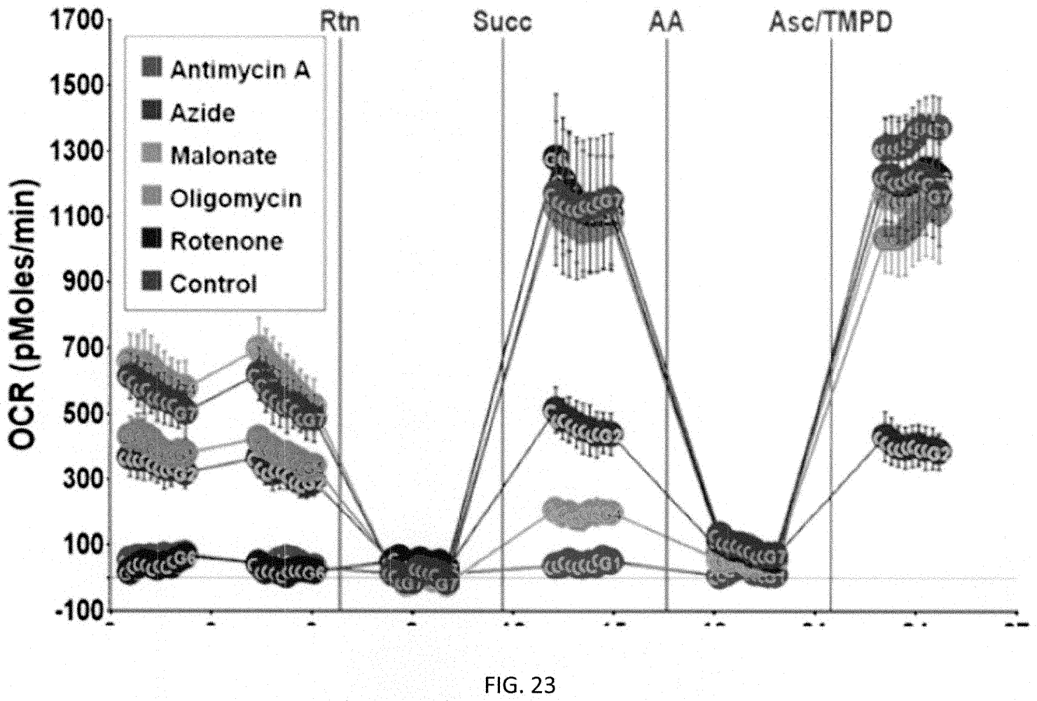

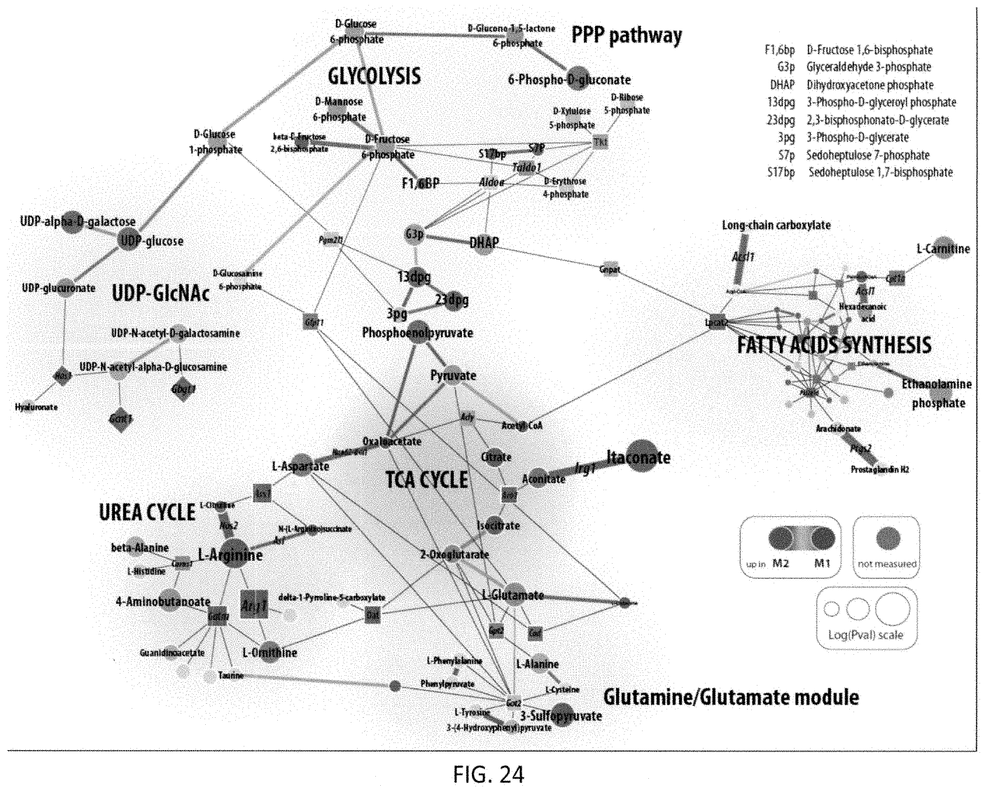

A substantial amount of functional evidence suggesting that itaconate can affect macrophage's inflammatory response was been collected. The present data show that anti-inflammatory properties of itaconate are associated with regulation of succinate via timely inhibition of succinate dehydrogenase (Sdh), also known as complex II of electron transport chain. It is presently thought that such inhibition is possible due to structural similarity between itaconate and succinate (see e.g., FIG. 17B), suggesting a competitive inhibition mechanism. Consistently, complex II mediated mtROS production is reduced in the presence of itaconate in the context of activating macrophages (see e.g., FIG. 19) and in the context of ischaemia-reperfusion injury (see e.g., FIG. 22). This example describes how to dissect interactions between itaconate and Sdh, as well as itaconate and electron transfer chain, on the molecular level to firmly establish the basis of the functional effects of itaconate.

(I) Test Direct Inhibitory Effect of Itaconate on Sdh and Icl Activity.

First, the exact nature of the effect of the itaconate on the human and mouse Sdh will be confirmed and its ability to convert succinate to fumarate in enzyme assay. A study has reported direct inhibitory effect of itaconate, characterized as competitive inhibition with affinity slightly bigger for itaconate than that of malonate, another structural mimetic of succinate well known to be competitive inhibitor of Sdh. However, it is believed that no discussion or further work on itaconate-Sdh interaction has been done, and it has never been independently reproduced. Classical biochemistry assay will be used to determine inhibitory characteristics of itaconate towards Sdh.

Specifically, inhibitory properties of itaconate on succinate dehydrogenase will be determined in mouse BMDM using Complex II Enzyme Activity Microplate Assay Kit (Abcam). This systems allows in-well purification of the Sdh from cell culture homogenates. Sdh will be purified from cell lysates and its activity will be analyzed in colorimetric assay based on the production of ubiquinol by the SDH enzyme that is coupled to the reduction of the dye DCPIP (2,6-diclorophenolindophenol) according to following reaction:

Ubiquinol (QH2)+DCPIP (blue)→ubiquinone (Q)+DCPIPH2 (colorless).

Decrease in DCPIP absorbance at 600 nm will be measured. In order to check for another potential sources of hydrogen causing reduction of DCPIP than Sdh, malonate, a competitive inhibitor of Sdh, will be also analyzed.

To extend the results obtained in mouse BMDMs to human samples, the impact of itaconate on Sdh activity will be also determined in macrophages derived from human peripheral blood mononuclear cells (PBMCs). Briefly, white blood cell samples from anonymous donors will be obtained from Pheresis center in Barnes-Jewish Hospital. PBMCs will be isolated by ficoll gradient centrifugation. Subsequently, the Miltenyi Biotec MACS magnetic cell separation system will be used to isolate the CD14+monocyte fraction from the PBMCs. Isolated CD14+monocytes will then be cultured in the presence of colony stimulating factor 1(CSF-1) to differentiate them into macrophages.

Finally, in recent studies anti-bacterial role of itaconate has been widely discussed. The proposed mechanism of action is associated with inhibition of isocitrate lyase (Id), bacteria-specific enzyme of glyoxilate shunt. To that end, results from similar competitive inhibition assays will be reproduced using Id enzyme isolated from M.tuberculosis and from Salmonella. These data will allow for the assessment of the magnitude of itaconate effect on macrophages versus bacterial cells.

(II) Determine the Extent of Itaconate on the Mitochondrial Function by Measuring Oxygen Consumption Rate (OCR) and Extracellular Acidification Rate (ECAR).

Seahorse Bioscience Metabolic Analyzer is an analytical instrument that can simultaneously measure in vitro cellular oxygen consumption rate (OCR) and extracellular acidification rate (ECAR) of the cells as frequent as every 2-5 minutes. This allows one to assess in real time cellular functions such as TCA cycle (OCR is indicative of mitochondrial respiration) and glycolysis (ECAR measures rate of acid reflux—predominantly lactic acid formed via glycolytic metabolism). This instrument has become de facto standard for any modern study of metabolic rewiring in immune cells. Using Seahorse Analyzer, we, and other researchers in the field, have established that mitochondrial function is decreased in macrophages at 24 hours after LPS stimulation as well as the fact that glycolytic flux is increased.

Importantly, Seahorse Analyzer design allows to perform continuous OCR/ECAR measurements while adding different chemicals to cellular milieu. Hence, one can directly measure the degree of coupling between the electron transport chain and oxidative phosphorylation machinery and effects of different compounds on the OCR and ECAR. For instance, this allows to discriminate the inhibitors of complexes I, II or III (see e.g., FIG. 9). FIG. 9 shows representative experiments performed by Seahorse Bioscience, where mitochondria isolated from cells are treated consecutively with complex I inhibitor Rotenone at 7 minutes, complex II substrate Succinate at 12 minutes, complex III inhibitor Antimycin A and complex IV substrates Asc/TMPD. Accordingly, after treatment of normal mitochondria (brown curves on FIG. 9) with rotenone, OCR falls to nearly zero since ETC has been completely blocked. Upon subsequent supplementation with succinate, substrate of complex II (downstream of complex I), ETC electron flow is recovered, leading to rescue of the oxygen consumption, which is then blocked again by complex III inhibition. Finally, complex IV substrates supplementation leads to recovery of OCR.

If, however, mitochondria are initially loaded in the SeaHorse Analyzer in the presence of the complex II inhibitor malonate (light blue curve on the FIG. 9), the response to sequential treatment described above will be different. Specifically, upon succinate addition (minutes 12 to 16 on FIG. 9), rescue of the oxygen consumption will be incomplete (OCR does not go up as high) due to competition between malonate and succinate.

Accordingly, such OCR measurements in the bone-marrow derived macrophages will be performed before and after LPS stimulation, and in the presence and absence of itaconate pretreatment. This will allow for direct confirmation of the effect of the itaconate on the mitochondrial function.

(III) Test Effect of Itaconate on the Cellular and Mitochondrial ROS Production and Downstream Immunological Function

It is well known that complex I and III inhibitors can induce mitochondrial ROS production by redirecting electron flow towards oxygen species, which in turn can drive proinflammatory cytokine production. Intriguingly, complex II inhibitors were shown to play dual role, capable of both inhibiting and enhancing reactive oxygen species production depending on the direction of the electron flow in the ETC. The present data show (see e.g., FIG. 19B) that itaconate inhibits mitochondrial ROS production in LPS stimulated macrophages at 24 hour timepoint. In this part of the project the details of the ROS production in WT and Irg1 deficient macrophages will be investigated to establish detailed mechanistic picture of itaconate's role.

Three types of mitochondria-specific labels that distinguish respiring (Mitotracker deep red), total (Mitotracker green) and ROS-generating mitochondria (MitoSOX) will be used. The following will be performed:
Perform such ROS measurements in the bone-marrow derived macrophages after LPS stimulation, and in the presence and absence of itaconate pretreatment. This will allow for direct confirmation of the effect of the itaconate on the mitochondrial function during macrophage activation.
Perform ROS measurements as described above in the itaconate pre-treated macrophages in the absence of LPS stimulation. Malonate pretreated macrophages will be used as positive control for complex II inhibition.
Perform ROS measurements as described above in the Irg1^{-/-} macrophages.

These results allow for defining the relationship between itaconate and ROS production in detail. Furthermore, cellular ROS will be measured by staining cells with CelIROX dye for the final 30 mins of the LPS stimulation. Recent work has shown that inhibiting complex I in macrophages leads to inhibition of ROS production and reduced production of Ill b, further confirming relation between macrophage activation and electron transport chain (ETC).

Finally, given that itaconate and succinate have opposing effect on functioning of ETC, measurements will be performed of ROS production in different cultures supplemented with mixtures of succinate and itaconate at different proportions to observe direct competing effect on macrophage activation.

(IV) Interpretation

The above experiments comprise rigorous analysis of molecular mechanism of itaconate interaction with succinate dehydrogenase. Among described, the most challenging experiments are the ones related to mitochondrial ROS production measurements since reported magnitude of ROS upregulation in LPS stimulated macrophages is relatively modest. It is for this reason it is preferred to use multiple ROS readout to firmly establish the observed effects. It is known that while mitochondrial function is defective at 24 hours post LPS stimulation (as measured by oxygen consumption), oxygen consumption at early time points continues to be at the same level as prior the stimulation. This change might be associated with the reversal of the direction of electron flow in the ETC in early and late time points. Accordingly, investigation of details of the electron flow directionality in macrophage early and late after LPS stimulation may need to be performed. Seahorse Analyzer experiments with appropriate sequence of substrates and inhibitors can be used.

### Example 4: Characterize the Role of Itaconate in Macrophage Activation at a Systems Level

The timeline of metabolic rewiring during macrophage activation is highly orchestrated: immediate stimulation-induced upregulation of glycolysis is followed by NO production at ˜6 hours, with itaconate production kicking in at ˜8-10 hours post-stimulation. To investigate this cross-over point in details, the following will be performed: a) assemble comprehensive metabolic and transcriptional profiles of macrophage activation along the time course spanning 0-24 hours in both WT and Irg1^{-/-} genetic backgrounds; b) use integrated metabo-transcriptional network analysis pipeline currently developed to identify temporal sequence of events and critical pathways involved in metabolic rewiring; and c) validate analyses using pharmacological inhibition of critical pathways and targeted gene deletions.

Multidimensional process of macrophage activation is relatively well characterized on the level of signaling pathways and transcriptional regulators. Additionally, a number of studies described the details of epigenetic regulation, such as changes in histone modifications and transcription factor binding sites. However, metabolic rewiring was described to a much lower extent. This present work (2) was the first one to provide publicly accessible resource for broad coverage (˜500 metabolites) metabolomics profiles for activated and nonactivated macrophages. These data addressed 24 hour time point post stimulation with LPS+IFNg and IL-4.

In addition to providing unique data, a computational analysis pipeline focused on integrating metabolic and transcriptional data was developed. These two data types provide unique complementary outlook at the systems level changes underlying macrophage activation: regulation of enzymes can be assessed through transcriptional profiles and linked to changes in metabolites via corresponding reactions catalyzed by the enzymes. This strategy allows for the identification of subnetworks that are most differentially regulated between two conditions, thus zooming in on the most important metabolic nodes. It has been shown here, such analytical approach leads to identification of novel pathways, such as UDP-GlcNAc module (see e.g., FIG. 10), and validated them using appropriate pharmacological inhibitors. It is using this strategy itaconate-Irg1 was identified as one of the major metabo-transcriptional markers of macrophage activation (green box in FIG. 10) and determined Idh1-associated breakpoint in TCA cycle. In this part of the project, it is proposed to apply this integrated analysis pipeline to investigate the role of itaconate by comparing WT and Irg1^{-/-} macrophages.

(I) Collect Metabo-Transcriptional Profiles for Time Course of Macrophage Activation in WT and Irg1 KO Backgrounds

Newly generated Irg1 knock-out mouse will be used to obtain comparative transcriptional and metabolomics profiles. Since itaconate production begins at approximately 7 hours post-stimulation and reaches maximum at 9-10 hours, it is important to reconstruct the chronological context in which itaconate production occurs. Accordingly, profile bone-marrow derived macrophages at 2 hour interval are collected after stimulation with LPS (i.e. at 0, 2, 4, 6, 8, 10, 12, 16, and 24 h time points). Given the considerable amount of time point samples as well as biological replicates, it was decided to use the custom high-throughput RNA-seq protocol to streamline sample processing.

Specifically, 3′-end focused RNA-sequencing with a barcode-first strategy will be used that allows sample pooling at the cDNA stage, improving sensitivity and consistency between samples, and allowing construction of a high-throughput library from material extracted from cell lysates in a single well of a 96-well plate. Sequencing data will be analyzed using computational pipeline established in-house which includes read alignment by STAR aligner, counting with custom script based on ht-seq software and differential expression calling with DESeq2.

To obtain metabolic profiles, a non-targeted, flow-injection-analysis (FIA) mass spectrometry (MS) method will be used, enabling broad metabolite coverage between 50 and 1000 daltons, and yielding quantitative information on approximately ˜10000 MS spectral features. All steps of MS data processing will be performed with Matlab R2010b (The Mathworks, Natick) using functions native to the Bioinformatics, Statistics, Database, and Parallel Computing toolboxes.

(II) Carry Out Comprehensive Integrated Network Analysis of the Metabolic and Transcriptional Changes

First, a traditional pathway level analyses of the metabolomics and transcriptional datasets will be separately carried out. However, such approach inherently fails to leverage the notion of coherent changes between these regulatory levels, diminishing their power to identify important regulators of metabolic rewiring. The present network-based data integration approach is based on comparison of two states (such as unstimulated vs LPS stimulated macrophages) to identify most critical metabolite-enzymatic subnetwork controlling metabolic rewiring between such two states. In the course of the present work, computational algorithms have been optimized to be able to analyze multiple samples, as opposed to previous version where only pair-wise comparison could be carried out. Specifically, a global murine cellular reaction network (CRN) was used that connects ˜3000 metabolites and corresponding enzymes based on the latest edition of the KEGG database. Next, is characterizing individual metabolites and enzymes based on the degree of the mutual correlation between multiple samples and seeking for the most strongly weighted subnetwork (typically of the size ˜100 nodes) within the CRN accounting for both the nodal connectivity and the degree of differential expression of metabolites and enzymes in the network. This optimized approach will be used to comprehensively describe the high-dimensional time-course data obtained for the WT and Irg1^{-/-} macrophages and identify core subnetworks representing major metabolic modules of macrophage metabolic rewiring.

(III) Validate Major Regulatory Modules Controlling Timeline of Metabolic Rewiring

Two major approaches will be used to validate metabolic rewiring architecture emerging from the analyses above. First, labeling studies will be used to confirm that metabolic fluxes are redirected as predicted by the network analysis. Typically, in such experiments C13-labeled glucose or glutamine are used to establish major routes of the utilization of these metabolites by tracing the fate of C13 label. Considering labeling time of 4 hours, such labeling experiments can be performed at early or late phases of macrophage activation (e.g. from 1-5 hours and from 20-24 hours).

The second major approach involves targeting critical modules identified by network analysis. This can be done by perturbing relevant pathways via pharmacological inhibition or targeting most critical genes of the corresponding modules by shRNA knock-down in primary macrophages. Changes in typical markers of inflammation, such as cytokine production, iNOS upregulation, etc. can serve as read-out to determine relevance of the module.

Finally, the wealth of data obtained from these studies serve as excellent resource to study chronological details of metabolic rewiring in LPS-stimulated macrophages beyond involvement of itaconate.

### Example 5: Test how Pharmacologic Administration of Itaconate or Deletion of Irg1 Regulates Immune Responses and Modulates Disease In Vivo

The present data show that itaconate can be safely administered in vivo and its pretreatment results in decreased macrophage response to live Salmonella infection. This suggests that anti-inflammatory effect of itaconate is transferrable from in vitro models to in vivo settings. Indeed, the present data show that itaconate treated mice succumb to Salmonella infection faster than their untreated counterparts. Details of in vivo itaconate action in this model will be investigated and use Irg1^{-/-} mice to identify major mechanism of susceptibility, details of in vivo metabolic rewiring during inflammation, and test dominant role of macrophage-produced itaconate using newly generated Irg1^{fl/fl} mice.

In the context of immunity to bacterial infection, the present data show that itaconate production affects not only bacterial virulence but also modulates immune response of macrophages. Thus, it was asked what would be the effect of itaconate in the in vitro system that contains both live macrophages and bacteria: is it predominantly affecting macrophages or bacteria? To address this question, BMMDs were infected with Mycobacterium tuberculosis (Mtb) H37Rv strain (MOI of 1). Three treatment groups were set up: untreated macrophages, macrophages pretreated with interferon, and macrophages pretreated with interferon and then treated with itaconate. The infection was then allowed to proceed for 7 days. On day 7 post infection, serial dilutions of infected BMDM homogenates were plated on 7H11 agar plates and colony forming unit (CFU) determined.

FIG. 11 shows that itaconate treatment results in the increased bacterial burden in the cells suggesting that anti-inflammatory action of itaconate on macrophages plays dominant role even when live bacteria are present. Consistently, II1b levels in the supernatants are decreased in the presence of itaconate (data not shown). These results establish that in at least some bacterial infection contexts, itaconate significantly affects macrophages' ability to mount immune response as opposed to serving as direct anti-bacterial metabolite. Similar results were obtained in the context of bacterial infection with Salmonella Typhimurium.

### Example 6: Systemic Metabolic Changes in Macrophages Stimulated with LPS are Associated with Large Production of Itaconate, Structural Mimetic of Succinate

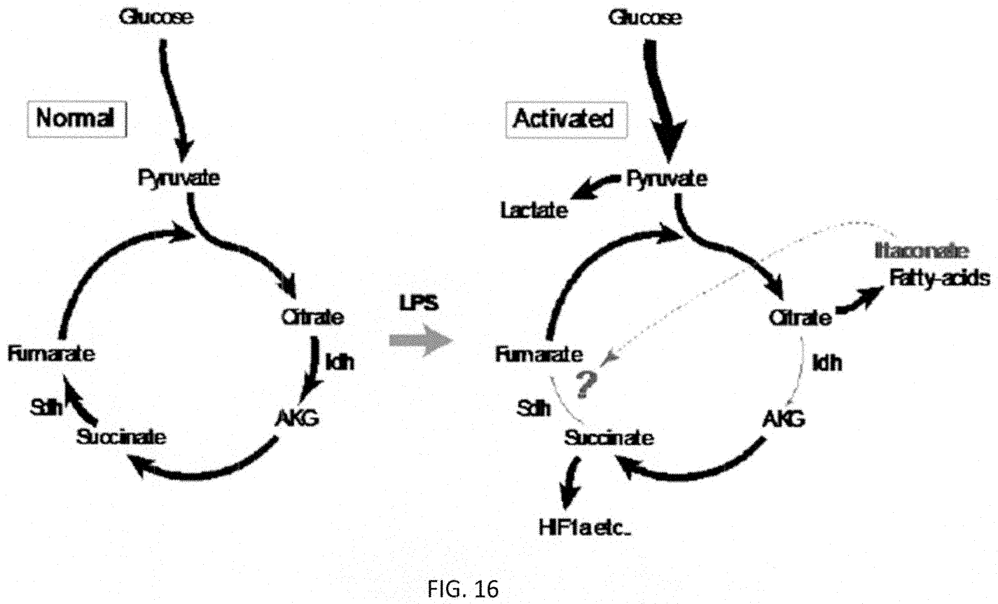

Metabolic rewiring of macrophages during inflammatory response (such as LPS stimulation) is characterized by two major features (see e.g., FIG. 16). First, immediately upon stimulation, macrophages and dendritic cells undergo transition in the glycolytic flux regulation comparable to Warburg effect observed in cancer cells: glycolysis is upregulated leading to increased production of pyruvate which is then converted to lactate rather than feeding into tricarboxylic acid (TCA) cycle. At the same time, normal mitochondrial functions are impaired through changes in the membrane potential and NAD/NADH ratio which is followed by emergence of so called anapleurotic TCA cycle—situation when individual TCA metabolites are used as building blocks and signaling molecules instead of partaking in the metabolic cycle. The studies have described one mechanism achieving anapleurosis by breaking metabolic flux of the TCA cycle at the transition between isocitrate and 2-oxoglutarate (see e.g., FIG. 16). Transcriptional levels and enzymatic activity of isocitrate dehydrogenase (Idh1) are significantly decreased in activated macrophages effectively stalling TCA cycle at this point. Metabolic flux is instead redirected towards production of the fatty acids and itaconate (five-carbon dicarboxylic acid), both major metabolic products secreted by activated macrophages (see e.g., FIG. 16). It is hypothesized that a second TCA cycle breakpoint leading to succinate accumulation is controlled by itaconate via inhibition of Sdh due to its structural similarity with succinate, substrate of Sdh.

A number of indirect evidence suggests a presence of a second breakpoint in the TCA cycle at the succinate to fumarate transition (see e.g., FIG. 16). First, succinate levels increase significantly in the activated macrophages, similar to citrate accumulation at the Idh1 breakpoint. Second, in labeling experiments using media containing C13-labeled glutamine, C13-label distribution differs between succinate and fumarate in activated macrophages but not in unstimulated macrophages indicating the change in the flux between fumarate and succinate. Finally, succinate accumulation can play specific functional role: externally added succinate enhances inflammatory responses (such as II1b production), highlighting importance of succinate as pro-inflammatory signal in macrophage activation.

Several major mechanisms of inflammatory succinate action were described to date. First, succinate accumulation was shown to lead to a number of pro-inflammatory effects via (1) direct inhibition of prolyl hydroxylase (PHD) enzymes by accumulated succinate leads to stabilization of HIF1a and activation of downstream transcriptional cascade, including IL1 b production; (2) direct action through dedicated G-protein coupled receptor—GPR91; and (3) protein succinylation targets some known enzymes such as glyceraldehyde 3-phosphate dehydrogenase (GAPDH) and malate dehydrogenease (MDH)). Conversely, active conversion of succinate to fumarate was shown to fuel mitochondrial ROS production in the ischaemia-reperfusion model outside the immune context. In this model, succinate accumulated during ischaemia stage is converted to fumarate via complex II of electron transport chain (another name for succinate dehydrogenase, Sdh) which feeds reverse flow of electron transport chain (ETC) and leads to production of mitochondrial ROS (mtROS) through complex I. This mode of succinate action, however, to date was not explored in the context of immune cell activation. As evident from the oxygen consumption rate (OCR) timecourse measurement, TCA cycle is actively working first several hours after stimulation and succinate is converted to fumarate, while only later TCA cycle switches to anapleurotic state and succinate is accumulated at approximately 6-8 hours post-stimulation, highlighting critical importance of TCA for early stages of macrophage activation. The exact mechanism leading to succinate accumulation is not known. Conceivably, this transition is achieved through timely inhibition of succinate to fumarate conversion by Sdh. Overall, control of Sdh activity presents critical regulatory point in metabolic rewiring of activating macrophages.

### Example 7: Itaconate Isa Succinate's Structural Mimetic with Potential Sdh Inhibitory Properties and Anti-Inflammatory Effect in the Context of Inflammasome Activation and Lps Stimulation of Macrophages

Chemicals structurally similar to succinate can serve as competitive inhibitors to succinate dehydrogenase (Sdh), typical example being malonate, a metabolite that is not naturally produced in immune cells. Intriguingly, itaconate—one of the most highly produced intracellular metabolites (see e.g., FIG. 17A), is a structural analog of succinate (see e.g., FIG. 17B). It is currently believed that no studies of the effect of itaconate on the immune cells themselves were carried out to date.

Given the magnitude of intracellular production of itaconic acid (see e.g., FIG. 17A), it was first tested to determine if itaconate was also produced extracellularly. Indeed, unlike succinate or other metabolites of TCA cycle, the concentration of itaconate in the supernatants of stimulated macrophages was significantly increased at 24 hours (data not shown).

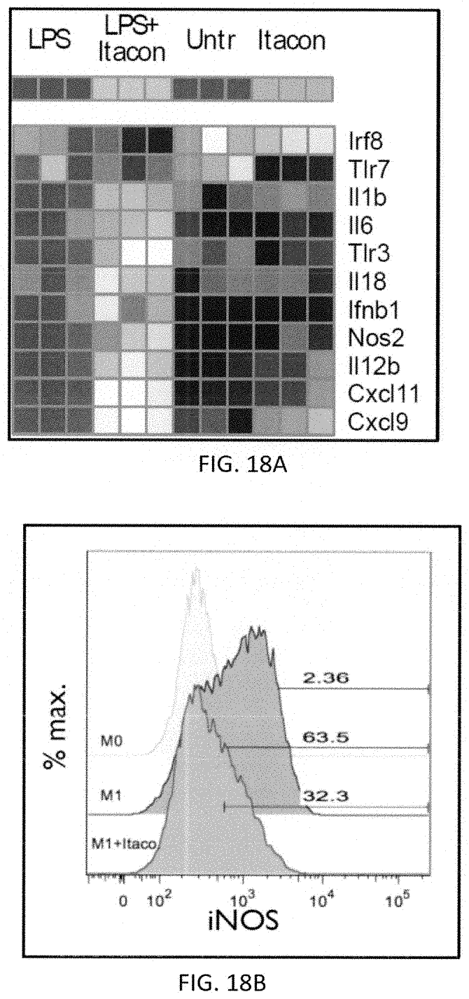

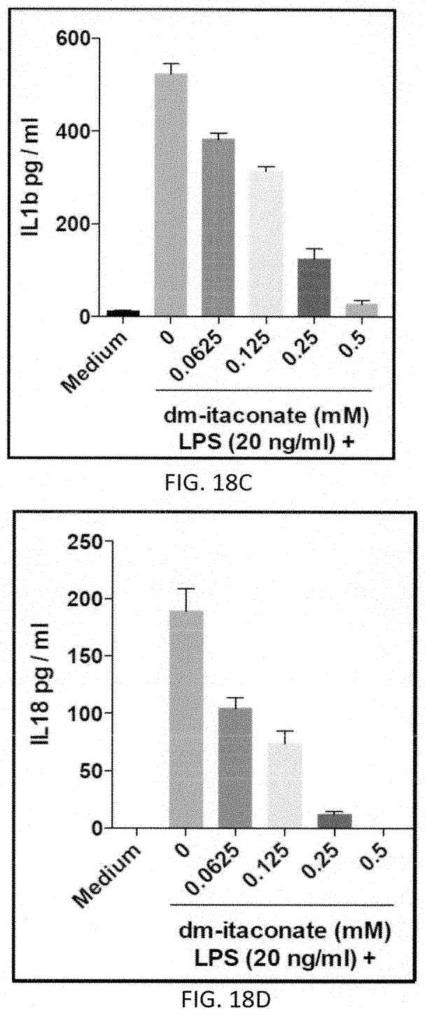

Similarly, transcriptional levels of Irg1, itaconate producing enzyme, were increased upon LPS stimulation more than 200-fold. Such characteristic regulation of both metabolite and its corresponding enzyme in activating macrophages is reminiscent of iNOS/NO and Cox2/PGE2 regulation and suggest broader role for the itaconate than previously ascribed anti-microbial function. This is particularly underscored by the fact that Irg1 is induced not only in response to TLR4 stimulation but also to TLR3 and interferon stimulations. To that end, effect of itaconate addition on the macrophage activation was evaluated. It was determined that itaconate (in its membrane permeable form—dimethyl-itaconate) is not toxic up to 0.5 mM concentration and bone-marrow derived macrophages pretreated with 0.25 mM itaconate were used for subsequent analysis. RNA-seq based transcriptional profiling of activated macrophages showed that in the presence of itaconate upregulation of inflammatory markers is significantly decreased compared to macrophages activated in the absence of itaconate. Such markers include iNos, II6, II1b, II18, Ifnb1 and II12b (see e.g., FIG. 18A). Pathway analysis also showed broad downregulation of immune activation pathways, highlighting broad anti-inflammatory effect of the itaconate pretreatment. Next, it was tested if transcriptional inhibitory effect was translated on the protein production level and assessed iNOS levels (see e.g., FIG. 18B) and II6 production levels (data not shown) in the LPS stimulated macrophages as well as II1b and II18 cytokine levels produced in the inflammasome activation by LPS+ATP (see e.g., FIG. 18C, FIG. 18D). It was found in all cases that addition of itaconate has dose-dependently inhibited production of corresponding inflammatory proteins. Thus, it was concluded that itaconate has broad anti-inflammatory effect in the context of macrophage activation by LPS or LPS+ATP.

### Example 8: Itaconate can Affect Macrophage Activation Through Mitochondrial Ros

Inflammasome activation is generally divided into two stages: first signal is associated with synthesis of pro-II1β, and the second signal ensures successful cleavage of the precursor to its final form, II1β that is then secreted. To evaluate which signal was affected by itaconate, a western blot of pro-II1β was obtained in the presence and absence of itaconate. It was observed that levels of pro-Ill b were significantly decreased (see e.g., FIG. 19A), suggesting that inflammasome priming is affected by the itaconate addition. We, thus, considered potential ways for itaconate to affect signal one. Mechanistically, inflammasome activation via LPS+ATP treatment has been linked with oxidative damage to the mitochondrial DNA through mtROS production. This could link itaconate's role as potential Sdh inhibitor and its anti-inflammatory effect via electron transport chain's role in mtROS production. Thus, whether itaconate reduces LPS-induced mtROS production was tested. Indeed, as can be seen from FIG. 19B, in the presence of itaconate LPS-induced upregulation of mtROS is impaired. Consistently, it has been previously reported that mtROS inhibition interferes with inflammasome priming (signal one), as opposed to the inflammasome assembly step (signal two).

### Example 9: Genetic Ablation of Irg1 Abrogates Itaconate Production and Leads to Succinate Accumulation Presumably by Relieving Sdh from Itaconate Inhibition

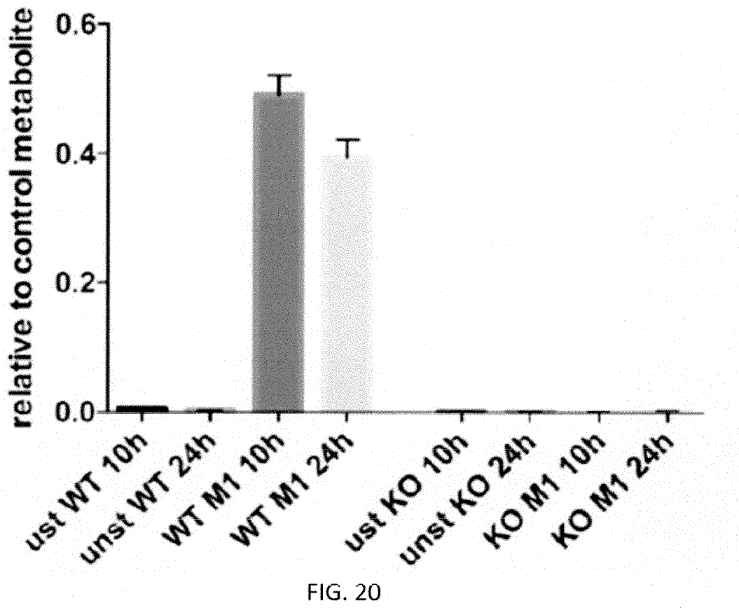

To further investigate regulatory role of itaconate a newly generated Irg1 knockout mouse that lacks production of Irg1 protein due to removal of first exon was characterized. Irg1 (Immune-responsive gene 1) is cis-aconitate carboxylase, mediating itaconate production from cis-aconitate and localized to mitochondria. Irg1 is significantly upregulated transcriptionally in response to a number of inflammatory stimuli including LPS, interferon, TNF, etc. First, to rule out alternative routes of itaconate generation, metabolic profiles for Irg1-deficient bone marrow derived macrophages stimulated with LPS were obtained. FIG. 20 shows that indeed, Irg1 is the only enzyme responsible for itaconate production in macrophages: deletion of Irg1 completely abrogates production of itaconate.

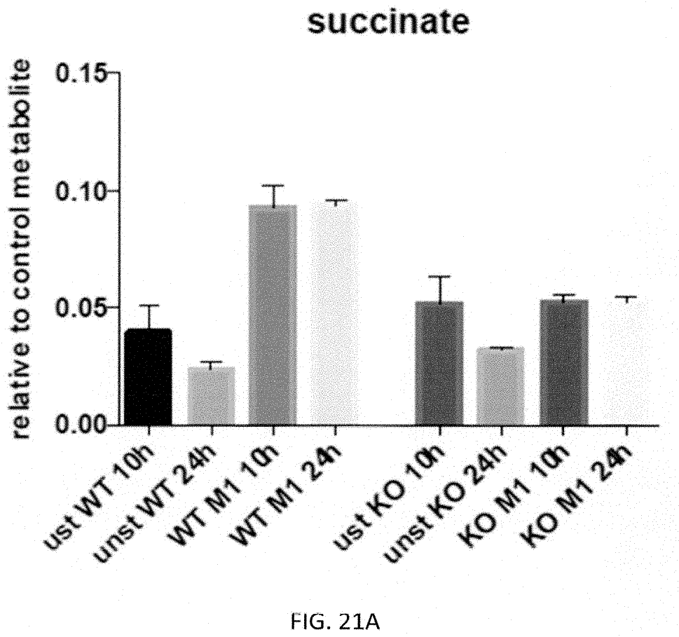

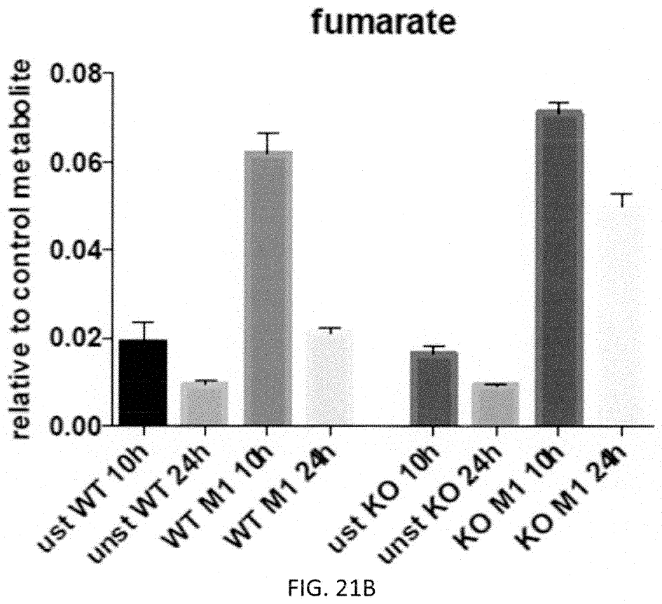

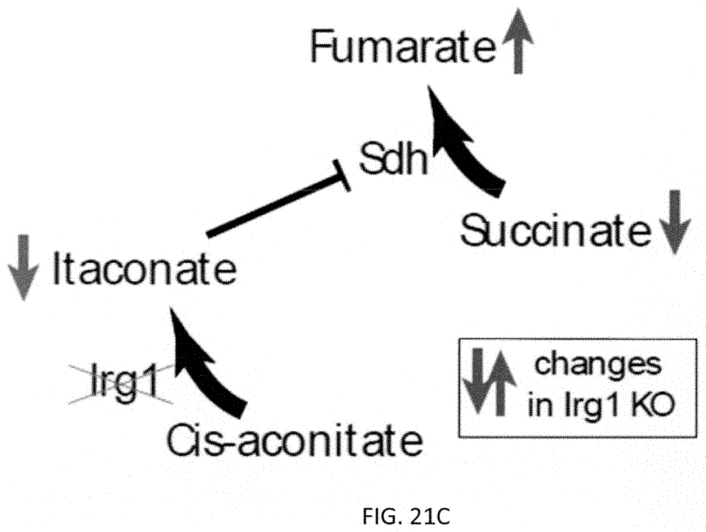

Previous studies focused on the function of itaconate-producing enzyme (Irg1) also found connection with reactive oxygen species production. It has been shown that knock-down of Irg1 was associated with defective tolerance to repeated LPS stimulation-cytokine production levels were higher in Irg1 knock-down cells upon second LPS exposure compared to control shRNA. The data show that itaconate accumulation is directly linked to accumulation of succinate—in the absence of itaconate production (i.e., in the Irg1^{-/-} macrophages), succinate levels do not increase after stimulation with LPS in contrast to normal macrophages at 10 and 24 h post stimulation (see e.g., FIG. 21A). Moreover, show that itaconate accumulation is directly linked to accumulation of succinate—in the absence of itaconate production (i.e., in the Irg1^{-/-} macrophages), succinate levels do not increase after stimulation with LPS in contrast to normal macrophages at 10 and 24 h post stimulation (see e.g., FIG. 21B), further supporting the role of itaconate as inhibitor of Sdh, since in the absence of itaconate production, Sdh would remain active and maintain active succinate conversion to fumarate (see e.g., FIG. 21C). Overall, these observations corroborate the hypothesis that itaconate serves as an endogenous regulator of succinate levels in the process of macrophage activation.

### Example 10: Itaconate Modulates Ros-Dependent Ischaemia-Reperfusion Injury Consistent with Complex II Inhibitory Properties

To ensure that observed relationship between itaconate, succinate dehydrogenase (Sdh), and mitochondrial ROS production is functionally important in vivo, an experiment in the in vivo mouse model of myocardial ischaemia-reperfusion injury was performed. Studies found that myocardial injury is caused by succinate driven release of mitochondrial ROS ultimately leading to cellular damage. In this model, the vein is occluded in the open heart surgery, which blocks the blood flow, creates hypoxic conditions and generally mimics situation of infarct. Then, after ˜30 mins the vein is released leading to blood flow recovery. The latter stage is called reperfusion and it has been established that myocardial injury happens during this stage due to active conversion of succinate to fumarate via Sdh which is accompanied by active mtROS generation. Succinate itself is accumulated during ischaemia via reverse action of Sdh. It is presently thought that myocardial damage can be significantly reduced by i.v. addition of dimethylmalonate during the ischaemia stage. Being structural mimetic of succinate, malonate inhibits complex II, which decreases succinate accumulation, thus decreasing mtROS production and extent of myocardial damage during reperfusion stage. A study to investigate the effects of itaconate and malonate and found that itaconate is also protective in the model to the same extent as malonate was performed. As can be seen on FIG. 22, addition of itaconate during ischaemia stage reduces infarct size as well as malonate does. This shows that itaconate can serve as an endogenous in vivo modulator of inflammatory responses via ROS production and Sdh regulation.

Additionally, evidence was collected that i.v. administration of itaconate significantly accelerates mortality from Salmonella infection in vivo.

### Example 11: Itaconate Suppresses Th17 Differentiation and Inhibits IL-17-Associated Autoimmune Inflammation In Vivo

The following example describes how itaconate suppresses Th17 differentiation.

As described herein, itaconate becomes highly upregulated both as an intracellular and extracellular metabolite in LPS-activated macrophages. It was then asked whether secreted itaconate might act in an autocrine manner on macrophages themselves. In view of itaconate's dramatic suppressive effects on activated macrophages, determination of its potential paracrine effect on other cells types was sought. One of the cells types that activated macrophages often encounter in vivo in secondary lymphoid organs and/or in affected tissues during numerous diseases in humans and rodents, are CD4 T cells. Activated myeloid cells, like macrophages and dendritic cells, can drive and/or promote the differentiation of naïve CD4 T cells into distinct T helper (Th) subsets with effector functions that are commonly defined by the production of a specific array of cytokines. Of these subsets, the so-called Th17 subset (Th17 cells) and its signature cytokines including IL-17, GM-CSF, IL-22 and others, have been most frequently associated with autoimmune pathology in diseases as diverse as inflammatory bowel disease, psoriasis, multiple sclerosis, rheumatoid arthritis and others. Importantly, there is currently no cure for these diseases and the available treatments have partial and often only short-term success.

For these reasons investigation of the effect of itaconate on the differentiation of Th17 cells in vitro (FIG. 25) and Th17 associated autoimmune inflammation in vivo (FIG. 26) was studied.

(I) Itaconate Suppresses Th17 Differentiation.

Purified naïve CD4 T cells (from C56BL/6 mice) were differentiated into Th17 cells under the typical Th17 polarizing cytokine conditions (IL-6, IL-1, IL-23, TGFβ, anti-IFNγ, anti-IL-4) in presence or absence of DI (dimethyl itaconate; FIG. 25A-FIG. 25C), itaconic or related acids (FIG. 25D-FIG. 25F). FIG. 25A-FIG. 25F shows that itaconate, both in its acid and ester form, can suppress Th17 differentiation in a structure-specific manner.

It was shown that DI has a moderate dose-dependent cytotoxic effect (see e.g., FIG. 25A). FIG. 25A shows the frequency of live CD4 T cells as determined by flow cytometry on day 4 after Th17 polarization in presence of the indicated doses of DI.

FIG. 25B is a flow cytometry plot and bar graph showing DI dose-dependently inhibits the frequency of IL-17-producing cells. (left) Representative flow cytometry plot showing how the frequency of IL-17+ CD4 T cells was determined (top left+right quadrat). (right) Bar graph shows the frequency IL-17+ cells determined under different DI doses.

FIG. 25C is a series of plots showing DI inhibits also the secretion of IL-17 (left) and GM-CSF (right) in the supernatant of CD4 T cells. On day 4 cells were collected, washed, and re-stimulated in equal numbers for each condition with anti-CD3 and anti-CD28. After 24 h supernatants were collected and assayed for IL-17 and GM-CSF by ELISA.

FIG. 25D is a series of bar graphs showing itaconic acid is not cytotoxic under Th17 conditions and dose-dependently inhibits Th17 differentiation. (Left) bar graph shows the frequency of live CD4 T cells determined as in A; (right) bar graph shows the frequency of IL-17+ cells determined as in FIG. 25B.

FIG. 25E are chemical structures of itaconic acid and structurally similar dicarboxylic acids.

FIG. 25F is a series of bar graphs showing inhibition of Th17 by itaconic acid is structure specific; (left) bar graph shows the frequency of live CD4 T cells determined as in A on day 3 post polarization. (right) bar graph shows the frequency of IL-17-producing cells determined as in A. All acids were used at 5 mM.

(II) In Vivo Administration of Itaconate Inhibits IL-17-Associated Autoimmune Inflammation In Vivo.

FIG. 26A-FIG. 26B is a series of graphs and images showing that in vivo administration of itaconate inhibits IL-17-associated autoimmune inflammation in vivo.

FIG. 26A is a plot showing DI-treatment diminished in vivo systemic IL-17 production. Circulating serum IL-17 levels were determined by ELISA 48 h after anti-CD3-induced inflammation in mice treated or not with the indicated doses of DI. Briefly, mice were received intraperitoneal (ip) injections of 4 mg (n=10) or 20 mg (n=5) DI or vehicle (PBS; shown as 0; n=10) 3 h prior (−3 h) to an anti-CD3 injection (given at 0 h ip) and every 20 h thereafter until mice were analyzed at 48 h.

FIG. 26B is a series of histology images showing DI administration in vivo limits IL-17 associated pathology in imiquimod (IMQ)-induced psoriasis model. IMQ cream (5%) was applied on mouse ear skin for 7 days and skin pathology was assessed on day 8. Mice received 20 mg DI ip. (DI+IMQ) or vehicle ip (PBS; IMQ) was administered one day prior to IMQ application and every day thereafter until day 7. DI group: received 20 mg DI as above but no IMQ; naïve mice received vehicle as above but no IMQ. Images shows hematoxylin and eosin stains of mouse ear skin sections of the groups described above. Administration of DI prevents dermal edema that occurs during IMQ-induced skin inflammation.

1. A method of treatment of a disease, disorder, or condition associated with an inflammatory response or an immune response in a subject in need thereof comprising:
administering a therapeutically effective amount of an immunomodulatory agent comprising itaconate, malonate, or a derivative thereof to the subject;
wherein,
the therapeutically effective amount reduces or prevents inflammation or an immune response.

2. The method of claim 1, wherein the immune response is an LPS-mediated immune response.

3. (canceled)

4. The method of claim 1, wherein itaconate, malonate, or a derivative thereof comprises a compound of formula I: 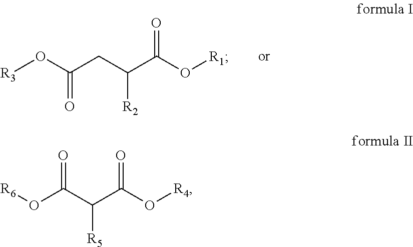
or a pharmaceutically acceptable salt thereof, including all tautomers and stereoisomers thereof wherein,
R₁ is hydrogen, unsubstituted or substituted alkyl; unsubstituted or substituted alkenes; or unsubstituted or substituted alkynes;
R₂ is hydrogen, unsubstituted or substituted alkyl; unsubstituted or substituted alkenes; or unsubstituted or substituted alkynes;
R₃ is hydrogen, unsubstituted or substituted alkyl; unsubstituted or substituted alkenes; or unsubstituted or substituted alkynes;
R₄ is hydrogen, unsubstituted or substituted alkyl; unsubstituted or substituted alkenes; or unsubstituted or substituted alkynes;
R₅ is hydrogen, unsubstituted or substituted alkyl; unsubstituted or substituted alkenes; or unsubstituted or substituted alkynes; and
R₆ is hydrogen, unsubstituted or substituted alkyl; unsubstituted or substituted alkenes; or unsubstituted or substituted alkynes;
wherein,
R₁, R₂, R₃, R₄, and R₅ is optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; a C₂₋₆ cycloalkyl optionally containing unsaturation or one oxygen or nitrogen atom; straight chain or branched C₁₋₁₀alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl;
the unsubstituted phenyl ring or substituted phenyl ring is optionally substituted with one or more groups independently selected from the group consisting of hydroxyl; C₁₋₁₀alkyl hydroxyl; amine; C₁₋₁₀carboxylic acid; C₁₋₁₀carboxyl; straight chain or branched C₁₋₁₀alkyl, optionally containing unsaturation; straight chain or branched C₁₋₁₀alkyl amine, optionally containing unsaturation; a C₂₋₆cycloalkyl optionally containing unsaturation or one oxygen or nitrogen atom; straight chain or branched C₁₋₁₀alkyl amine; heterocyclyl; heterocyclic amine; aryl comprising a phenyl; and heteroaryl containing from 1 to 4 N, O, or S atoms; and
the unsubstituted heterocyclyl or substituted heterocyclyl is optionally substituted with one or more groups independently selected from the group consisting of hydroxyl; C₁₋₁₀alkyl hydroxyl; amine; C₁₋₁₀carboxylic acid; C₁₋₁₀ carboxyl; straight chain or branched C₁₋₁₀alkyl, optionally containing unsaturation; straight chain or branched C₁₋₁₀alkyl amine, optionally containing unsaturation; a C₂₋₆cycloalkyl optionally containing unsaturation or one oxygen or nitrogen atom; heterocyclyl; straight chain or branched C₁₋₁₀alkyl amine; heterocyclic amine; and aryl comprising a phenyl; and heteroaryl containing from 1 to 4 N, O, or S atoms.

5. The method of claim 4, wherein
R₁ is H or CH₃;
R₂ is CH₂, CH₃, or H;
R₃ is H or CH₃;
R₄ is H or CH₃;
R₅ is H or CH₃; and
R₆ is H or CH₃.

6. The method of claim 1, wherein the itaconate or malonate, or a derivative thereof is selected from the group consisting of: 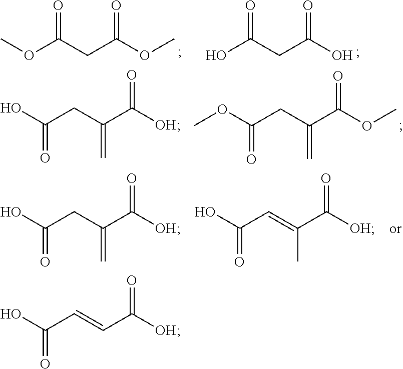
or
a pharmaceutically acceptable salt thereof, including all tautomers and stereoisomers.

7. The method of claim 1, wherein the immunomodulatory agent downregulates proinflammatory pathways or upregulate Phase II conjugation, glutathione conjugation, or biological oxidations.

8. The method of claim 1, wherein the disease, disorder, or condition is selected from the group consisting of ischemia-reperfusion injury or an immune response, wherein the immune response is an autoimmune response or a lipopolysaccharide (LPS)-mediated immune response; or the immunomodulatory agent interferes with (i) activation of pro-inflammatory macrophages, (ii) ROS-related oxidative stress, (iii) inflammatory T cell response (iv) pathogenic adaptive immune response; (v) IL-17, or (vi) GM-CSF-production; and TNF-α production is substantially unaffected.

9. (canceled)

10. The method of claim 1, wherein the disease, disorder, or condition is associated with increased expression or increased secretion of Casp1, HIF-1α, pro-IL-1β, ASC, NLRP3, NOS2, iNOS, NO, IL6, IFNβ1, IL-12p70, IL-6, IL-1β, IL-12β, GM-CSF, IL-17, or IL-18.

11. The method of claim 1, wherein the immunomodulatory agent inhibits:
inflammasome function;
conversion of succinate to fumarate;
succinate dehydrogenase (Sdh);
IL-17-associated autoimmune inflammation; or
frequency of IL-17-producing cells.

12. The method of claim 1, wherein the immunomodulatory agent reduces, suppresses, or down regulates pro-IL-1β, ASC, NLRP3, iNos, IL6, IL1b, IL18, IFNB1, IL12b, mROS, succinate, iNOS, HIF-1α, Nos2, or Th17 differentiation.

13. The method of claim 1, wherein the immunomodulatory agent suppresses or inhibits secretion or production of IL-1β, IL-6, IL-17, IL-18, IL-12p70, NO, or GM-CSF.

14. The method of claim 1, wherein the immunomodulatory agent:
modulates expression of II1b, II18, P2rx7, Casp1, or an inflammasome adapter Pycard (ASC);
attenuates hypoxia-induced increase in ROS generation and protects against hypoxia-induced cell death; or
regulates succinate levels, mitochondrial respiratory rate, and inflammatory cytokine production during macrophage activation.

15. The method of claim 1, wherein the immunomodulatory agent is formulated as a pharmaceutical composition comprising one or more pharmaceutically acceptable diluents or carriers.

16. The method of claim 1, wherein
the disease, disorder, or condition is associated with a lipopolysaccharide (LPS)-mediated immune response and
the immunomodulatory agent
(i) suppresses a lipopolysaccharide (LPS)-mediated immune response;
(ii) modulates II1b, II18, P2rx7, Casp1, or an inflammasome adapter Pycard (ASC);
(iii) impairs LPS-induced upregulation of mtROS; or
(iv) downregulates LPS-induced expression of HIF-1α; or
(v) promotes the glutathione-mediated anti-oxidant pathway.

17. The method of claim 1, wherein the disease, disorder, or condition is selected from one or more of the following: adult and juvenile Still disease; asthma; allergy; Alzheimer's disease; age-related macular degeneration; antisynthetase syndrome; autoinflammatory disease; autoimmune disease; autoimmune response; Behcet disease; Blau syndrome; cancer; cardiovascular infarction; chronic infantile neurological cutaneous and articular (CINCA) syndrome; chronic recurrent multifocal osteomyelitis; cinca syndrome; classic autoinflammatory diseases; cryopyrin-associated autoinflammatory syndromes (CAPS); deficiency in IL-1 receptor antagonist (DIRA); diabetes mellitus; Erdheim-Chester syndrome (histiocytosis); extrapulmonary tuberculosis; familial atypical mycobacteriosis; familial cold autoinflammatory syndrome (FCAS); gastric cancer Risk after H. pylori Infection; Guillain-Barré syndrome; Hashimoto's thyroiditis; heart failure; hepatic fibrosis; Huntington's disease; hyper IgD syndrome (HIDS); hypoxia; ischaemia-reperfusion; immunodeficiency 29; inflammation; inflammation by HIV; inflammatory bowel disease (IBD); macrophage activation syndrome (MAS); mycobacteriosis; Miller-Fisher syndrome; Muckle-Wells syndrome (MWS); multiple sclerosis (MS); neonatal-onset multisystem inflammatory disease (NOMID); neuropathic pain; N syndrome; osteoarthritis; osteoporosis; Periodontal Disease; plaque psoriasis; psoriatic arthritis; periodic fever, aphthous stomatitis, pharyngitis, adenitis syndrome (PFAPA); postmyocardial infarction heart failure; psoriasis; recurrent idiopathic pericarditis; recurrent pericarditis; relapsing chondritis; relapsing-remitting multiple sclerosis; rheumatoid arthritis (RA); Sapho Syndrome; Schnitzler syndrome; secondary progressive multiple sclerosis; septic shock; smoldering myeloma; systemic sclerosis; Sweet syndrome; synovitis, acne, pustulosis, hyperostosis, osteitis (SAPHO); systemic juvenile rheumatoid arthritis; familial Mediterranean fever (FMF); pyogenic arthritis; pyoderma gangrenosum, acne (PAPA); TNF receptor-associated periodic syndrome (TRAPS); type 2 diabetes; urate crystal arthritis (gout); urticarial vasculitis; or vitiligo.

18. The method of claim 17, wherein
(i) the disease, disorder, or condition is cardiovascular infarction or ischaemia-reperfusion in heart, kidney, or brain and the immunomodulatory agent protects against hypoxia-induced cell death; or
(ii) the disease, disorder, or condition is psoriasis and the immunomodulatory agent prevents skin edema and reduces inflammation.

19. (canceled)

20. The method of claim 1, wherein
the immune modulatory agent reduces tissue injury by a reduction in mitochondrial reactive oxygen species (mROS); or
the immunomodulatory agent suppresses immune response or inhibits IL-17-associated autoimmune inflammation.

21. A method to reduce tissue injury during cardiovascular infarction in a subject in need thereof, the method comprising administering to the subject an immunomodulatory agent comprising itaconate, malonate, or a derivative thereof.

22. The method of claim 21, wherein itaconate, malonate, or a derivative thereof comprises a compound of formula I: 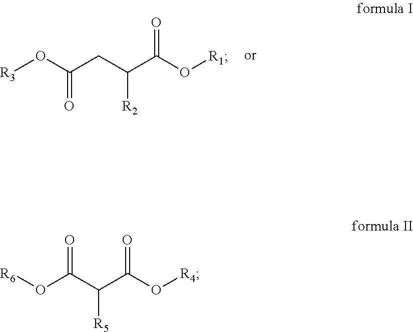
or a pharmaceutically acceptable salt thereof, including all tautomers and stereoisomers thereof wherein,
R1 is hydrogen, unsubstituted or substituted alkyl; unsubstituted or substituted alkenes; or unsubstituted or substituted alkynes;
R2 is hydrogen, unsubstituted or substituted alkyl; unsubstituted or substituted alkenes; or unsubstituted or substituted alkynes;
R3 is hydrogen, unsubstituted or substituted alkyl; unsubstituted or substituted alkenes; or unsubstituted or substituted alkynes;
R4 is hydrogen, unsubstituted or substituted alkyl; unsubstituted or substituted alkenes; or unsubstituted or substituted alkynes;
R5 is hydrogen, unsubstituted or substituted alkyl; unsubstituted or substituted alkenes; or unsubstituted or substituted alkynes; and
R6 is hydrogen, unsubstituted or substituted alkyl; unsubstituted or substituted alkenes; or unsubstituted or substituted alkynes;
wherein,
R1, R2, R3, R4, and R5 is optionally substituted with one or more groups independently selected from the group consisting of hydroxyl; C1-10alkyl hydroxyl; amine; C1-10carboxylic acid; C1-10carboxyl; straight chain or branched C1-10alkyl, optionally containing unsaturation; a C2-6 cycloalkyl optionally containing unsaturation or one oxygen or nitrogen atom; straight chain or branched C1-10alkyl amine; heterocyclyl; heterocyclic amine; and aryl comprising a phenyl; heteroaryl containing from 1 to 4 N, O, or S atoms; unsubstituted phenyl ring; substituted phenyl ring; unsubstituted heterocyclyl; and substituted heterocyclyl;
the unsubstituted phenyl ring or substituted phenyl ring is optionally substituted with one or more groups independently selected from the group consisting of hydroxyl; C1-10alkyl hydroxyl; amine; C1-10carboxylic acid; C1-10carboxyl; straight chain or branched C1-10alkyl, optionally containing unsaturation; straight chain or branched C1-10alkyl amine, optionally containing unsaturation; a C2-6 cycloalkyl optionally containing unsaturation or one oxygen or nitrogen atom; straight chain or branched C1-10alkyl amine; heterocyclyl; heterocyclic amine; aryl comprising a phenyl; and heteroaryl containing from 1 to 4 N, O, or S atoms; and
the unsubstituted heterocyclyl or substituted heterocyclyl is optionally substituted with one or more groups independently selected from the group consisting of hydroxyl; C1-10alkyl hydroxyl; amine; C1-10carboxylic acid; C1-10carboxyl; straight chain or branched C1-10alkyl, optionally containing unsaturation; straight chain or branched C1-10alkyl amine, optionally containing unsaturation; a C2-6 cycloalkyl optionally containing unsaturation or one oxygen or nitrogen atom; heterocyclyl; straight chain or branched C1-10alkyl amine; heterocyclic amine; and aryl comprising a phenyl; and heteroaryl containing from 1 to 4 N, O, or S atoms.

23. The method of claim 21, wherein the cardiovascular infarction area is reduced in size.


SEQUENCE LISTING (2 sequences)

SEQ ID NO: 1 - DNA, 23 bp, Artificial Sequence
  synthetic primer
cctacacgacgctcttccgatct

SEQ ID NO: 2 - DNA, 22 bp, Artificial Sequence
  synthetic linker
agatcggaagagcacacgtctg